(19) United States
(12) Patent Application Publication
(10) Pub. No.: US 2022/0186325 A1
(43) Pub. Date: Jun. 16, 2022

(54) SYSTEMS FOR SAMPLE ANALYSIS

(71) Applicant: Biomeme, Inc., Philadelphia, PA (US)

(72) Inventors: Marc DeJohn, Philadelphia, PA (US); Christopher Cox, Philadelphia, PA (US); Tom Welsh, Philadelphia, PA (US); Luke Gary, Philadelphia, PA (US); Alexia Quinn, Philadelphia, PA (US)

(21) Appl. No.: 17/554,181

(22) Filed: Dec. 17, 2021

Related U.S. Application Data
(63) Continuation of application No. PCT/US2020/038159, filed on Jun. 17, 2020.
(60) Provisional application No. 62/862,938, filed on Jun. 18, 2019.

(51) Int. Cl. C12Q 1/6888; B01L 3/00; G01N 21/64; G01N 1/44

(52) U.S. Cl. CPC C12Q 1/6888; B01L 3/502715; G01N 21/6428; G01N 1/44; G01N 2201/021; B01L 2300/0654; B01L 2300/0832; B01L 2300/0609; G01N 2021/6439; B01L 2300/1805

(57) ABSTRACT

The present disclosure provides devices, systems, methods for processing and/or analyzing a biological sample. An analytic device for processing and/or analyzing a biological sample may comprise a moving carriage. The analytic device may be portable. The analytic device may receive instructions for performing an assay from a mobile electronic device external to a housing of the analytic device.

### CROSS-REFERENCE

This application is a continuation of International Application No. PCT/US20/38159 filed Jun. 17, 2020, which claims the benefit of U.S. Provisional Patent Application No. 62/862,938, filed Jun. 18, 2019, each of which is entirely incorporated herein by reference.

### BACKGROUND

Nucleic acid-based amplification reactions are now widely used in research and clinical laboratories for the detection of genetic and infectious diseases. However, the devices and systems used to perform these amplification reactions may be bulky. This may limit their portability and use in the field. Furthermore, the need to ship samples to a laboratory for analysis may result in contamination from handling, sample degradation, and delays in obtaining the results of the assay. Moreover, some assays may require manual labor for sample preparation. Sample preparation can include removal of the caps of containers containing samples or reagents, determining a type or measuring an amount of reagent necessary to process a sample, and pipetting one or more reagents together.

### SUMMARY

Recognized herein is a need for portable analytic device for analyzing biological samples. The present disclosure provides a portable analytic device and methods for amplifying and/or detecting analytes from a sample in a substantially lab-free environment. The results of such an assay may be directed to a user, such as a subject. The user may then use the results of the assay for various purposes, including identifying a disease (e.g., an infectious disease or contamination).

Also recognized herein are various limitations associated with some methods for sample preparation. Sample preparation may be labor intensive, requiring multiple steps and operator involvement. Some labor-intensive steps can vary from one assay to another, leading to operator error and possible contamination of the sample by the operator. Manual processing of a sample may carry the risk of exposure of the operator to potentially dangerous biological chemicals.

In view of certain limitations of current methods for sample preparation, recognized herein is a need for a system that can automate the sample handling and sample preparation process for analytical procedures.

In an aspect, the present disclosure provides a portable analytic device for processing a biological sample, comprising: a housing with a volume that is less than about 1,500 cubic centimeters; at least one monolithic heating block within the housing, wherein the at least one monolithic heating block comprises a plurality of recesses configured to receive a plurality of assay tubes, wherein an assay tube of the plurality of assay tubes comprises the biological sample; at least one heating unit in thermal communication with the at least one monolithic heating block, which at least one heating unit provides thermal energy to the assay tube through the at least one monolithic heating block; at least one light path comprising an excitation filter and an emission filter, wherein the at least one light path is configured to provide excitation energy from an excitation source to the assay tube; and a power supply disposed within the housing, the power supply configured to provide power to the at least one heating unit and the excitation source.

In some embodiments, the portable analytic device further comprises a processing unit comprising a circuit within the housing, wherein the processing unit is configured to direct the excitation source to provide the excitation energy. In some embodiments, the processing unit is operatively coupled to the at least one heating unit and the excitation source, and wherein the processing unit is configured to communicate with a mobile electronic device external to the housing. In some embodiments, the processing unit is configured to: receive instructions from the mobile electronic device external to the housing for processing the biological sample in the at least one of the two more assay tubes; and in response to the instructions, (i) direct the at least one heating unit to provide thermal energy to the at least one monolithic heating block to provide heat to the assay tube, and (ii) direct the excitation source to provide the excitation energy. In some embodiments, the instructions comprise a temperature of the at least one heating unit and/or a duration that the at least one heating unit is held at the temperature. In some embodiments, the portable analytic device further comprises a communication unit that provides wireless communication between the processing unit and the mobile electronic device. In some embodiments, the at least one monolithic heating block comprises a plurality of heating subunits each comprising a recess of the plurality of recesses, wherein a heating subunit of the plurality of heating subunits comprises a first opening disposed on a first side of the heating subunit for permitting the excitation energy to pass to the biological sample within the assay tube, and a second opening on a second side of the heating subunit to permit optical detection of emission energy from the biological sample within the assay tube. In some embodiments, the at least one monolithic heating block comprises a material having a specific heat capacity at 25 degrees Celsius of less than about 0.5 Joule/(gram×degrees Celsius). In some embodiments, the material is selected from the group consisting of aluminum, glass, iron, nickel, zinc, copper, brass, silver, and any combination thereof. In some embodiments, a volume of the material used to construct the at least one monolithic heating block is less than about 0.5 cubic centimeters. In some embodiments, the at least one heating unit comprises a resistive heater. In some embodiments, the at least one heating unit is (i) thermally cured to the at least one monolithic heating block, or (ii) soldered to the at least one monolithic heating block. In some embodiments, the at least one light path comprises one or more light pipes to convey the excitation energy from the excitation source to the assay tube. In some embodiments, the one or more light pipes comprise a first end comprising a single pipe, a second end comprising two or more pipes, and a branching portion therebetween. In some embodiments, the excitation source comprises one or more light emitting diodes (LEDs). In some embodiments, the one or more LEDs comprise single-color LEDs. In some embodiments, the one or more LEDs comprise a plurality of LEDs, and each of the plurality of LEDs is configured to emit a different wavelength of the excitation energy. In some embodiments, the portable analytic device further comprises a cooling unit disposed within the housing, which cooling unit reduces the thermal energy from the assay tube. In some embodiments, the cooling unit comprises one or more fans, wherein the one or more fans are configured to generate negative pressure adjacent to the assay tube to evacuate heat adjacent to the assay tube to an exterior of the housing. In some embodiments, the portable analytic device further comprises an optical detector disposed within the housing, the optical detector configured to detect emission energy from the biological sample within the assay tube. In some embodiments, the material has a thermal conductivity of at least about 100 Watts per meter per Kelvin.

In another aspect, the present disclosure provides a portable analytic device for processing a biological sample, comprising: a housing; at least one monolithic heating block within the housing, wherein the at least one monolithic heating block comprises a plurality of recesses configured to receive a plurality of assay tubes, wherein an assay tube of the plurality of assay tubes comprises the biological sample; at least one heating unit in thermal communication with the at least one heating block, which at least one heating unit provides thermal energy to the assay tube through the at least one monolithic heating block; a movable carriage comprising an optical filter, wherein the movable carriage is configured to translate to bring the optical filter in alignment with a light path that provides excitation energy from an excitation source to the assay tube; and a power supply disposed within the housing, the power supply configured to provide power to the at least one heating unit, the movable carriage, and the excitation source.

In some embodiments, the portable analytic device further comprises a processing unit comprising a circuit within the housing, wherein the processing unit is configured to (i) direct the movable carriage to translate and/or (ii) direct the excitation source to provide the excitation energy. In some embodiments, the processing unit is operatively coupled to the at least one heating unit and/or the excitation source, and wherein the processing unit is configured to communicate with a mobile electronic device external to the housing. In some embodiments, the processing unit is configured to: receive instructions from the mobile electronic device external to the housing for processing the biological sample in the assay tube; and in response to the instructions, (i) direct the at least one heating unit to provide thermal energy to the at least one monolithic heating block to provide heat to the assay tube, and (ii) direct the excitation source to expose the assay tube to excitation energy. In some embodiments, the instructions comprise a temperature of the at least one heating unit and/or a duration that the at least one heating unit is held at the temperature. In some embodiments, the portable analytic device further comprises a communication unit that provides wireless communication between the processing unit and the mobile electronic device. In some embodiments, the actuator comprises a motor. In some embodiments, each of the one or more light paths comprises one or more light pipes to convey the excitation energy from the excitation source to the assay tube. In some embodiments, the one or more light pipes comprise a first end comprising a single pipe, a second end comprising two or more pipes, and a branching portion therebetween. In some embodiments, the portable analytic device further comprises a cooling unit disposed within the housing, which cooling unit reduces the thermal energy from the assay tube. In some embodiments, the cooling unit comprises one or more fans, wherein the one or more fans are configured to generate negative pressure adjacent to the assay tube to evacuate heat adjacent to the assay tube to an exterior of the housing. In some embodiments, the portable analytic device further comprises an optical detector disposed within the housing, the optical detector configured to detect emission energy from the biological sample within the assay tube. In some embodiments, the optical filter is an emission filter. In some embodiments, the optical filter is an excitation filter. In some embodiments, the portable analytic device further comprises an emission filter.

In another aspect, the present disclosure provides a method for analyzing a biological sample, comprising: (a) activating a portable analytic device comprising: (i) a housing with a volume that is less than about 1,500 cubic centimeters; (ii) at least one monolithic heating block within the housing, wherein the at least one monolithic heating block comprises a plurality of recesses configured to receive a plurality of assay tubes, wherein an assay tube of the plurality of assay tubes comprises the biological sample; (iii) at least one heating unit in thermal communication with the at least one monolithic heating block, which at least one heating unit provides thermal energy to the assay tube through the at least one monolithic heating block; (iv) at least one light path comprising an excitation filter and an emission filter, wherein the at least one light path is configured to provide excitation energy from an excitation source to the assay tube; and (v) a power supply disposed within the housing, the power supply configured to provide power to the at least one heating unit and the excitation source; (b) receiving by the processing unit instructions from the mobile electronic device external to the housing for processing the biological sample in the assay tube; (c) in response to the instructions, directing the at least one heating unit to provide thermal energy to the monolithic heating block to provide heat to the biological sample within the assay tube; and (d) upon moving the movable carriage to the first position corresponding to the assay tube, directing the excitation source to expose the biological sample within the assay tube to excitation energy through the light path

In another aspect, the present disclosure provides a method for analyzing a biological sample, comprising: (a) activating a portable analytic device comprising: (i) a housing; (ii) at least one monolithic heating block within the housing, wherein the at least one monolithic heating block comprises a plurality of recesses configured to receive a plurality of assay tubes, wherein an assay tube of the plurality of assay tubes comprises the biological sample; (iii) at least one heating unit in thermal communication with the at least one monolithic heating block, which at least one heating unit provides thermal energy to the assay tube through the monolithic heating block; (iv) an excitation source configured to provide excitation energy; (v) a movable carriage comprising an excitation filter and an emission filter, wherein the movable carriage is configured to translate to bring the excitation filter and the emission filter to a first position in alignment with a light path that provides excitation energy from the excitation source to the assay tube; (vi) a power supply disposed within the housing, the power supply configured to provide power to the at least one heating unit, the movable carriage, and the excitation source; and (vii) a processing unit comprising a circuit within the housing, wherein the processing unit is configured to communicate with a mobile electronic device external to the housing; (b) receiving by the processing unit instructions from the mobile electronic device external to the housing for processing the biological sample in the assay tube; (c) in response to the instructions, directing the at least one heating unit to provide thermal energy to the monolithic heating block to provide heat to the biological sample within the assay tube; and (d) upon moving the movable carriage to the first position corresponding to the assay tube, directing the excitation source to expose the biological sample within the assay tube to excitation energy through the light path.

In some embodiments, moving the movable carriage to the first position corresponding to the assay tube comprises aligning the light path with the assay tube. In some embodiments, the optical filter comprises an emission filter. In some embodiments, the optical filter comprises an excitation filter. In some embodiments, the portable analytic device further comprises an emission filter. In some embodiments, the method further comprises, subsequent to (d), detecting emission from the biological sample within the assay tube, which emission is indicative of a presence or absence, or a relative amount, of a target molecule within the biological sample. In some embodiments, the movable carriage comprises a plurality of light paths. In some embodiments, the portable analytic device further comprises an actuator for moving the movable carriage from the first position to a second position. In some embodiments, in the first position, the light path is aligned with the assay tube and capable of directing the excitation source to expose the biological sample within the assay tube to a first excitation energy; and in the second position, a second light path of a plurality of light paths is aligned with the assay tube and capable of directing the excitation source to expose the biological sample within the assay tube to a second excitation energy. In some embodiments, the first excitation energy has a first wavelength, and the second excitation energy has a second wavelength. In some embodiments, the method further comprises receiving instructions at the processing unit from the mobile electronic device, the instructions comprising at least one temperature at which the at least one monolithic heating block is maintained. In some embodiments, the method further comprises extracting from the biological sample one or more nucleic acids. In some embodiments, the biological sample comprises one or more members selected from the group consisting of a blood sample, a plant sample, a water sample, a soil sample, and a tissue sample. In some embodiments, the biological sample contains or is suspected of containing a target nucleic acid molecule, and wherein the instructions comprise a target temperature(s) and number of heating and cooling cycles for conducting a nucleic acid amplification reaction on the target nucleic acid molecule, under conditions sufficient to yield amplification product(s) indicative of a presence or relative amount of the target nucleic acid molecule. In some embodiments, the method further comprises a data exchange unit that communicates with the mobile electronic device, wherein the data exchange unit (i) receives the instructions from the mobile electronic device, or (ii) provides results to the mobile electronic device upon processing the biological sample.

In another aspect, the present disclosure provides a sample processing system, comprising: a first fluid flow path; at least two multi-directional pumps comprising a first pump and a second pump in fluid communication with the first fluid flow path, and wherein the first pump and the second pump are configured to subject fluid in the first fluid flow path to flow along a first direction and a second direction, which second direction is different than the first direction; a dock configured to reversibly engage with a cartridge comprising a second fluid flow path in fluid communication with one or more reagents, wherein the dock is configured to bring the first fluid path in fluid communication with the second fluid flow path subsequent to engagement with the cartridge; and a third pump in fluid communication with the cartridge, which third pump is configured to dry at least one chamber within the cartridge.

In some embodiments, the third pump is a diaphragm pump. In some embodiments, the third pump is a unidirectional pump. In some embodiments, the first fluid flow path does not include any valves. In some embodiments, the sample processing system further comprises a controller operatively coupled to the at least two multi-directional pumps, wherein the controller is configured to direct the at least two multi-directional pumps to subject the fluid in the first fluid flow path to flow along the first direction and the second direction. In some embodiments, the sample processing system further comprises a lid comprising a body configured to come in contact with the cartridge when the dock has reversibly engaged with the cartridge, wherein the lid is coupled to a housing comprising the first fluid flow path and the at least two multi-directional pumps, and wherein the lid is configured to move towards the housing from (i) a first position in which the body contacts the cartridge, to (ii) a second position in which the first fluid path is brought in fluid communication with the second fluid flow path. In some embodiments, the lid is configured to rotate relative to the housing. In some embodiments, each of the at least two multi-directional pumps is configured to supply positive pressure and negative pressure in the first fluid flow path. In some embodiments, each of the at least two multi-directional pumps is configured to subject the fluid in the first fluid flow path to flow along a first direction and a second direction. In some embodiments, the sample processing system further comprises a fourth pump configured to come in fluid communication with the second fluid flow path when the dock has reversibly engaged with the cartridge. In some embodiments, the fourth pump is a peristaltic pump. In some embodiments, each of the at least two multi-directional pumps is a peristaltic pump. In some embodiments, the third pump is a peristaltic pump. In some embodiments, the at least one chamber is a waste chamber. In some embodiments, the sample processing system further comprises a conduit connecting the at least one chamber and the third pump, wherein the conduit comprises a valve. In some embodiments, the conduit comprises or is coupled to a pressure sensor to monitor pressure of the third pump.

In another aspect, the present disclosure provides a method for processing a sample, comprising: activating a system comprising (i) a first fluid flow path, (ii) at least two multi-directional pumps comprising a first pump and a second pump in fluid communication with the first fluid flow path, and wherein the first pump and the second pump are configured to subject fluid in the first fluid flow path to flow along a first direction and a second direction, which second direction is different than the first direction, (iii) a dock configured to reversibly engage with a cartridge, and (iv) a third pump in fluid communication with the cartridge, which third pump is configured to dry at least one chamber within the cartridge; engaging the dock with the cartridge comprising a second fluid flow path in fluid communication with one or more reagents, wherein subsequent to engagement of the dock with the cartridge, the first fluid path is in fluid communication with the second fluid flow path; and using the system and the one or more reagents to process the sample.

In some embodiments, the method further comprises removing the cartridge from the docket subsequent to processing the sample. In some embodiments, the sample is a biological sample.

In another aspect, the present disclosure provides a system for sample processing, comprising: a sample chamber comprising a filter configured to capture one or more nucleic acid molecules from a sample in the sample chamber; a funnel situated within the sample chamber, which funnel is configured to prevent transfer of the sample from the sample chamber to an environment external to the sample chamber; a well fluidly coupled to the sample chamber by a first conduit, the well configured to contain a reagent; a fluid flow unit in fluid communication with the first conduit, wherein the fluid flow unit is configured to subject the reagent to flow from the well to the sample chamber; one or more assay tubes, wherein a assay tube of the one or more assay tubes is fluidly coupled to the sample chamber via a second conduit; and a controller coupled to the fluid flow unit, wherein the controller is configured to receive instructions from a mobile electronic device for processing of the sample, and in accordance with the instructions, (i) direct the fluid flow unit to subject the reagent to flow from the well along the first conduit to the sample chamber, to provide a solution comprising the reagent and the one or more nucleic acid molecules in the sample chamber, and (ii) direct the fluid flow unit to subject the solution to flow from the sample chamber along the second conduit to the one or more assay tubes, such that the assay tube receives at least a portion of the solution.

In some embodiments, the funnel is configured to prevent liquid splashing from the sample chamber. In some embodiments, the funnel is configured to allow the reagent to flow through the funnel and into the sample chamber. In some embodiments, the system further comprises a second fluid flow unit fluidly coupled to and disposed downstream of the one or more assay tubes. In some embodiments, the second fluid flow unit is fluidly connected to the one or more assay tubes by a third conduit. In some embodiments, the assay tube comprises a cap, and wherein the third conduit between the assay tube and the second fluid flow unit is disposed in the cap. In some embodiments, the second fluid flow unit is configured to provide negative pressure to draw fluid from the sample chamber to at least one of the one or more assay tubes. In some embodiments, the second fluid flow unit is configured to provide positive pressure to the sample chamber to generate bubbles in the sample, thereby subjecting the sample to mixing. In some embodiments, the second fluid flow unit is fluidly coupled to the atmosphere. In some embodiments, the system further comprises a waste chamber fluidly coupled to the sample chamber by a fourth conduit. In some embodiments, the system further comprises a third fluid flow unit disposed along the fourth conduit between the waste chamber and the sample chamber. In some embodiments, the third fluid flow unit is configured to draw the sample from the sample chamber to the waste chamber. In some embodiments, the sample is drawn from the sample chamber to the waste chamber through the filter, thereby capturing the one or more nucleic acids from the sample in the filter. In some embodiments, the waste chamber comprises a vent plug, which vent plug swells when in contact with liquid and seals the waste chamber. In some embodiments, the sample chamber further comprises a sample chamber cap. In some embodiments, the sample chamber cap comprises a vent plug, which vent plug swells when in contact with liquid and seals the sample chamber. In some embodiments, the system further comprises a valve disposed along the first conduit between the well and the sample chamber. In some embodiments, the valve is disposed upstream of the fluid flow unit along the first conduit. In some embodiments, the system further comprises a plurality of wells, including the well. In some embodiments, the system further comprises a plurality of valves, wherein a valve of the plurality of valves is disposed along the first conduit between the plurality of wells and the sample chamber. In some embodiments, the reagent is a buffer that is selected from the group consisting of lysis buffer, wash buffer, a drying agent, and an elution buffer. In some embodiments, at least one of the sample chamber, the well, and the waste chamber comprises a seal. In some embodiments, the seal comprises at least one layer. In some embodiments, the at least one layer comprises polypropylene, adhesive, or aluminum. In some embodiments, the assay tube comprises a cap, and at least a portion of the second conduit between the sample chamber and the assay tube is disposed in the cap. In some embodiments, an end of the second conduit along an inner surface of the cap comprises a tip. In some embodiments, at least a portion of the second conduit is disposed in the tip. In some embodiments, a cross-sectional area of the second conduit decreases along an axial length of the tip. In some embodiments, the one or more assay tubes comprise a plurality of assay tubes, and wherein a cross-sectional area of a portion of the second conduit in the tip is different in at least two of the plurality of assay tubes. In some embodiments, an end of the third conduit along an inner surface of the cap comprises a molecular sieve. In some embodiments, the molecular sieve is porous. In some embodiments, the molecular sieve is permeable to a gas. In some embodiments, the molecular sieve is hydrophobic. In some embodiments, the cap extends into the assay tube. In some embodiments, the cap extends into the assay tube to a depth that determines a maximum working volume of the assay tube. In some embodiments, the depth of the cap is different than another depth of another cap extending into another assay tube of the one or more assay tubes. In some embodiments, the cap is removably coupled to the assay tube. In some embodiments, the assay tube comprises one or more pairs of primers for performing an assay to detect a target nucleic acid molecule. In some embodiments, the assay is polymerase chain reaction. In some embodiments, the system further comprises a heater in thermal communication with the sample chamber, wherein the heater is configured to subject a sample in the assay tube to heating. In some embodiments, the heater is configured to subject the sample to heating as part of one or more heating and cooling cycles. In some embodiments, the fluid flow unit is a pump or a compressor. In some embodiments, the fluid flow unit comprises one or more pumps. In some embodiments, the one or more pumps include a first pump and a second pump, wherein the first pump is configured to subject the reagent to flow from the well to the sample chamber, and wherein the second pump is configured to subject the solution to flow from the sample chamber to the one or more assay tubes. In some embodiments, the fluid flow unit comprises one or more compressors. In some embodiments, the system further comprises a sample processing unit comprising a dock, wherein the sample processing unit comprises the fluid flow unit, wherein the well and the sample chamber are included in a sample processing cartridge, and wherein the dock is configured to receive the cartridge to bring the well and the sample chamber in fluid communication with the fluid flow unit. In some embodiments, the controller is configured to come in wireless communication with the mobile electronic device. In some embodiments, the fluid flow unit comprises or is coupled to a pressure sensor.

In another aspect, the present disclosure provides a system for sample processing, comprising: a sample chamber comprising a filter configured to capture one or more nucleic acid molecules from a sample in the sample chamber; a well fluidly coupled to the sample chamber by a first conduit, wherein the first conduit is connected to a needle situated within the well, and wherein the well is configured to contain a reagent; a fluid flow unit in fluid communication with the first conduit, wherein the fluid flow unit is configured to subject the reagent to flow from the well to the sample chamber; one or more assay tubes, wherein a assay tube of the one or more assay tubes is fluidly coupled to the sample chamber via a second conduit; and a controller coupled to the fluid flow unit, wherein the controller is configured to receive instructions from a mobile electronic device for processing of the sample, and in accordance with the instructions, (i) direct the fluid flow unit to subject the reagent to flow from the well along the first conduit to the sample chamber, to provide a solution comprising the reagent and the one or more nucleic acid molecules in the sample chamber, and (ii) direct the fluid flow unit to subject the solution to flow from the sample chamber along the second conduit to the one or more assay tubes, such that the assay tube receives at least a portion of the solution.

In some embodiments, the needle comprises a groove, which groove is configured to drain the reagent.

In another aspect, the present disclosure provides a system for processing and analyzing a chemical or biological sample, comprising: a sample preparation unit configured to reversibly receive a sample preparation cartridge and process the chemical or biological sample within the sample preparation cartridge; an analysis unit configured to analyze at least one analyte within the chemical or biological sample processed by the sample preparation cartridge; and a controller operatively coupled to the sample preparation unit and the analysis unit, wherein the controller is configured to receive one or more instructions from a mobile electronic device for: (i) processing the chemical or biological sample with the sample preparation unit or analyzing the at least one analyte within the chemical or biological sample processed by the sample preparation cartridge, and (ii) in response to the one or more instructions, (1) directing the sample preparation unit to process the chemical or biological sample, or (2) directing the analysis unit to analyze the at least one analyte. In some embodiments, the sample preparation unit and the analysis unit are in a same housing.

In another aspect, the present disclosure provides a system for sample processing, comprising: a sample chamber configured to retain a solution; one or more assay tubes, wherein an assay tube of the one or more assay tubes is fluidly coupled to the sample chamber via a conduit, and wherein the conduit comprises a swellable particle; a fluid flow unit in fluid communication with the conduit; and a controller coupled to the fluid flow unit, wherein the controller is configured to direct the fluid flow unit to subject the solution to flow from the sample chamber along the conduit to the one or more assay tubes, such that the assay tube receives at least a portion of the solution and the swellable particle swells within the conduit.

In some embodiments, the sample chamber comprises a filter configured to capture one or more nucleic acid molecules from a sample in the sample chamber. In some embodiments, the system further comprises a well fluidly coupled to the sample chamber by an additional conduit, wherein the well is configured to contain a reagent. In some embodiments, the fluid flow unit is in fluid communication with the additional conduit, and wherein the fluid flow unit is configured to subject the reagent to flow from the well to the sample chamber. In some embodiments, the controller is further configured to direct the fluid flow unit to subject the reagent to flow from the well along the additional conduit to the sample chamber, to provide the solution comprising the reagent and the one or more nucleic acid molecules in the sample chamber. In some embodiments, the controller is configured to receive instructions from a mobile electronic device. In some embodiments, the assay tube comprises a cap, and at least a portion of the conduit between the sample chamber and the assay tube is disposed in the cap. In some embodiments, the at least the portion of the conduit comprises the swellable particle. In some embodiments, the swellable particle has a first cross-section, and wherein subsequent to the assay tube receiving the at least the portion of the solution, the swellable particle swelled to a second cross-section. In some embodiments, the first cross-section of the swellable particle is smaller than a cross-section of the conduit. In some embodiments, the first cross-section of the swellable particle is at least about 0.2 millimeters. In some embodiments, the cross-section of the conduit is at least about 0.5 millimeters. In some embodiments, the second cross-section of the swellable particle is at least about two times the first cross-section of the swellable particle. In some embodiments, the swellable particle is supported by an inner surface of the conduit. In some embodiments, the conduit further comprises a support in between the swellable particle and an inner surface of the conduit, and wherein the swellable particle is supported by the support. In some embodiments, the swellable particle is configured to swell to seal the conduit to block fluid flow in the conduit. In some embodiments, the swellable particle is a hydrogel particle. In some embodiments, the hydrogel particle comprises a polymeric material. In some embodiments, the polymeric material is sodium polyacrylate, polyacrylamide, or a functional derivative thereof.

In another aspect, the present disclosure provides a method for sample processing, comprising: (a) providing (i) a sample chamber comprising a solution and (ii) one or more assay tubes fluidly coupled to the sample chamber via a conduit, wherein the conduit comprises a swellable particle; and (b) using a fluid flow unit to subject the solution to flow from the sample chamber along the conduit to the one or more assay tubes, such that the assay tube receives at least a portion of the solution and the swellable particle swells within the conduit.

In some embodiments, the sample chamber and the one or more assay tubes are contained in a sample preparation cartridge. In some embodiments, the sample preparation cartridge further comprises a well fluidly coupled to the sample chamber by an additional conduit, wherein the well contains a reagent. In some embodiments, the fluid flow unit is in fluid communication with the additional conduit. In some embodiments, the method further comprises, prior to (b), using the fluid flow unit to subject the reagent to flow from the well to the sample chamber. In some embodiments, the assay tube comprises a cap, and at least a portion of the conduit between the sample chamber and the assay tube is disposed in the cap. In some embodiments, the at least the portion of the conduit comprises the swellable particle. In some embodiments, the swellable particle has a first cross-section, and wherein subsequent to the assay tube receiving the at least the portion of the solution, the swellable particle swelled to a second cross-section. In some embodiments, the first cross-section of the swellable particle is smaller than a cross-section of the conduit. In some embodiments, the first cross-section of the swellable particle is at least about 0.2 millimeters. In some embodiments, the cross-section of the conduit is at least about 0.5 millimeters. In some embodiments, the second cross-section of the swellable particle is at least about two times the first cross-section of the swellable particle. In some embodiments, the swellable particle is supported by an inner surface of the conduit. In some embodiments, the conduit further comprises a support in between the swellable particle and an inner surface of the conduit, and wherein the swellable particle is supported by the support. In some embodiments, the swellable particle swells to seal the conduit to block fluid flow in the conduit. In some embodiments, the swellable particle is a hydrogel particle. In some embodiments, the hydrogel particle comprises a polymeric material. In some embodiments, the polymeric material is sodium polyacrylate, polyacrylamide, or a functional derivative thereof.

In another aspect, the present disclosure provides a device, comprising: an assay tube; and a cap configured to be inserted into the assay tube, wherein the cap comprises a first conduit and a second conduit that are configured to come in fluid communication with the assay tube when the cap is inserted into the assay tube, wherein the first conduit is configured to supply a solution into the assay tube and the second conduit is configured to permit a gas within the assay tube to flow out of the assay tube, and wherein the first conduit comprises a swellable particle configured to swell upon the first conduit supplying the solution into the assay tube.

In some embodiments, the second conduit comprises a porous medium, which porous medium is configured to prevent the solution from entering the second conduit. In some embodiments, the porous medium is a molecular sieve. In some embodiments, the swellable particle has a first cross-section, and wherein the swellable particle is configured to swell to a second cross-section. In some embodiments, the first cross-section of the swellable particle is smaller than a cross-section of the first conduit. In some embodiments, the first cross-section of the swellable particle is at least about 0.2 millimeters. In some embodiments, the cross-section of the first conduit is at least about 0.5 millimeters. In some embodiments, the second cross-section of the swellable particle is at least about two times the first cross-section of the swellable particle. In some embodiments, the swellable particle is supported by an inner surface of the first conduit. In some embodiments, the first conduit further comprises a support in between the swellable particle and an inner surface of the first conduit, and wherein the swellable particle is supported by the support. In some embodiments, the swellable particle is configured to swell to seal the first conduit to block fluid flow in the first conduit. In some embodiments, the swellable particle is a hydrogel particle. In some embodiments, the hydrogel particle comprises a polymeric material. In some embodiments, the polymeric material is sodium polyacrylate, polyacrylamide, or a functional derivative thereof.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1B show various views of a housing for a portable analytic device for analyzing a biological sample. FIG. 1C shows a lid of a housing for a portable analytic device, the lid having a bendable comb capable of applying pressure and/or heat to an assay tube inserted into the analytic device. FIG. 1D shows an example of a housing for a portable analytic device with the lid open.

FIG. 2 shows a perspective view of an internal mechanism for a portable analytic device for analyzing a biological sample.

FIGS. 3A-3B show various heating blocks for use in a portable analytic device.

FIG. 4 shows a rear view of an internal mechanism for a portable analytic device with a circuit board removed, thereby exposing fans of the internal mechanism.

FIG. 5A shows a rear view of an internal mechanism for a portable analytic device with a circuit board and fans removed, thereby exposing a moving carriage of the internal mechanism. FIG. 5B shows a deconstructed view of a moving carriage of the internal mechanism. FIG. 5C shows a front view of a moving carriage of the internal mechanism, the moving carriage having multiple light paths.

FIG. 6A shows a bottom view of a moving carriage of the internal mechanism, the bottom of the moving carriage having multiple optical filters, which may be offset from one another. FIG. 6B shows a circuit board having multiple excitation sources (e.g., LEDs), which are spaced to correspond to the offset of the optical filters shown in FIG. 6A.

FIG. 7 shows another example of a moving carriage, having optical components (e.g., emission filters, excitation filters, LEDs and/or dichroic beam splitters) that rotate using a pinion mechanism.

FIG. 8 shows rear view of an internal mechanism for a portable analytic device for analyzing a biological sample.

FIG. 9 shows an example portable analytic device having multiple heating blocks, and assay tubes inserted into the heating blocks.

FIG. 10 shows a flow chart of an example method of analyzing a biological sample using a portable analytic device of the present disclosure, such as the device of FIG. 2A.

FIG. 11 shows a computer system that is programmed or otherwise configured to implement methods provided herein.

FIG. 12A shows an example cartridge that can be inserted into the analytic device for sample testing. The cartridge can contain one or more reagents to be used for nucleic acid amplification (e.g., polymerase chain reaction (PCR)). FIG. 12B shows an example cartridge inserted into the housing of the analytic device.

FIG. 13 shows an example portable analytic device having multiple heating blocks, and assay tubes inserted into the heating blocks.

FIG. 14A shows a front view of a movable carriage inside an example portable device. FIG. 14B shows a side view of an example portable device. FIG. 14C shows an additional front view of the example movable carriage inside a portable device. FIG. 14D shows a back view of the example movable carriage.

FIG. 15 shows a zoom-in view of an example movable carriage having a circular (or wheel-shaped) component.

FIG. 16 shows a side view of the internal mechanism of an example movable carriage inside a portable analytic device.

FIG. 17 shows a side view of the internal mechanism of an example movable carriage inside a portable analytic device.

FIG. 18 shows a zoom-in view of an example optical system of the movable carriage.

FIG. 19A shows an alternative configuration of the optical system. FIG. 19B shows another alternative configuration of the optical system.

FIG. 20A shows a plurality of individual heating blocks (e.g., unconnected blocks or single blocks) installed on a circuit board of an analytic device. FIG. 20B shows a plurality of individual heating blocks (e.g., unconnected blocks or single blocks). FIG. 20C shows a monolithic heating block having a plurality of connected heating blocks installed on a circuit board of an analytic device. FIG. 20D shows a monolithic heating block having a plurality of connected heating blocks.

FIG. 21A shows an example data comparing peak heating rate of heating blocks in single block configuration and monolith configuration. FIG. 21B shows an example data comparing peak cooling rate of heating blocks in single block configuration and monolith configuration. FIG. 21C shows an example data comparing heating uniformity of heating blocks in single block configuration and monolith configuration. FIG. 21D shows an example data comparing cooling uniformity of heating blocks in single block configuration and monolith configuration. FIG. 21E shows an example data comparing high temperature uniformity of heating blocks in single block configuration and monolith configuration. FIG. 21F shows an example data comparing low temperature uniformity of heating blocks in single block configuration and monolith configuration. FIG. 21G shows an example data comparing high temperature accuracy of heating blocks in single block configuration and monolith configuration. FIG. 2111 shows an example data comparing low temperature accuracy of heating blocks in single block configuration and monolith configuration.

FIG. 22A shows a schematic of an example of an automated sample preparation system. FIG. 22B shows a schematic of an example of an automated sample preparation system. FIG. 22C shows a schematic of an example of an automated sample preparation system. FIG. 22D shows a schematic of an example of an automated sample preparation system. FIG. 22E shows a schematic of an example of an automated sample preparation system.

FIG. 23 shows a cross-sectional view of a sample chamber of a sample preparation cartridge.

FIG. 24A shows a cross-sectional view of an assay tube being filled in a dropwise fashion with sample drawn from the sample chamber. FIG. 24B shows a cross-sectional view of an assay tube filled with sample drawn from the sample chamber.

FIG. 25A shows strips of assay tube caps having various lengths (e.g., along a longitudinal axis of the assay tube), each cap comprising a channel through which a sample may be drawn into the assay tube. FIG. 25B shows strips of assay tube caps having various lengths (e.g., along a longitudinal axis of the assay tube), each cap comprising a channel through which sample is drawn into the assay tube.

FIG. 26 shows a flow chart of an example method of preparing a sample using a sample preparation device or system of the present disclosure.

FIG. 27 shows a sample preparation cartridge docked to an automated sample preparation device.

FIG. 28 shows a sample preparation cartridge with assay tubes docked to an analytic device capable of performing an assay (e.g., polymerase chain reaction and/or detection of a target nucleic acid) on the sample in the assay tube.

FIG. 29 shows an example of a sample preparation cartridge.

FIG. 30 shows an example of a sample preparation cartridge.

FIG. 31 shows an example cross-section view of the sample chamber connected to a snorkel.

FIG. 32A shows an example sample preparation device assembly. FIG. 32B shows an example sample preparation device assembly. FIG. 32C shows an example sample preparation device assembly.

FIG. 33A shows a zoom-in view of needles in a sample preparation cartridge. FIG. 33B shows an example sample preparation cartridge having a plurality of needles on a surface of a manifold of the sample preparation cartridge.

FIG. 34A shows an example sample preparation cartridge having a sample chamber and a funnel within the sample chamber. FIG. 34B shows a cross-section view of a sample chamber and a funnel inserted in the sample chamber.

FIG. 35 shows an example sample preparation cartridge having vent plugs installed.

FIG. 36 shows a top view of a manifold of an example sample preparation cartridge.

FIG. 37A shows a cross-sectional side view of an example sample preparation cartridge.

FIG. 37B shows a cross-sectional side view of an example sample preparation cartridge.

FIG. 38 shows an example configuration of a swellable particle loaded within an inflow conduit for sealing the conduit after liquid fill.

FIG. 39 shows two different example configurations of an inflow conduit loaded with a swellable particle.

FIG. 40A shows an example sample preparation cartridge having an array of assay tubes filled with liquid samples with gas bubbles (or air bubbles) near bottom of the assay tubes.

FIG. 40B shows an image of the same sample preparation cartridge of FIG. 40A after performing PCR or thermo-cycling.

FIG. 41A shows PCR results of samples within a sample preparation cartridge.

FIG. 41B shows PCR results of samples within a sample preparation cartridge

FIG. 42A shows an example sample preparation cartridge having an array of assay tubes filled with liquid samples with gas bubbles near bottom of the assay tubes.

FIG. 42B shows an image of the same sample preparation cartridge of FIG. 42A after performing PCR or thermo-cycling.

FIG. 43A shows PCR results of samples within a sample preparation cartridge

FIG. 43B shows PCR results of samples within a sample preparation cartridge

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides devices, systems, and methods for sample processing and/or analysis. An analytic device may be portable and may comprise a housing, a heating block heated by a heating unit that is configured to provide thermal energy to a sample container including a sample, and a light path to provide excitation energy from an excitation source to the sample. An analytic device may be configured to accept and/or communicate with a mobile electronic device. An analytic device may also comprise a movable carriage that comprises an optical filter and an excitation source and is configured to translate to bring the optical filter in alignment with the light path. The inclusion of a movable carriage may facilitate the production of a smaller and/or less expensive analytic device as one or more excitation sources, optical filters, and light paths of the movable carriage may be used to process and/or analyze multiple sample containers including multiple samples. An analytic device may be used to analyze a biological sample including, or suspected of including, one or more nucleic acid molecules to determine the presence or an amount of the one or more nucleic acid molecules.

The term “monolithic,” as used herein, generally refers to an element that is single piece. In some examples, a monolithic heating block is formed of a single-piece (or unitary) material. Analytic Devices

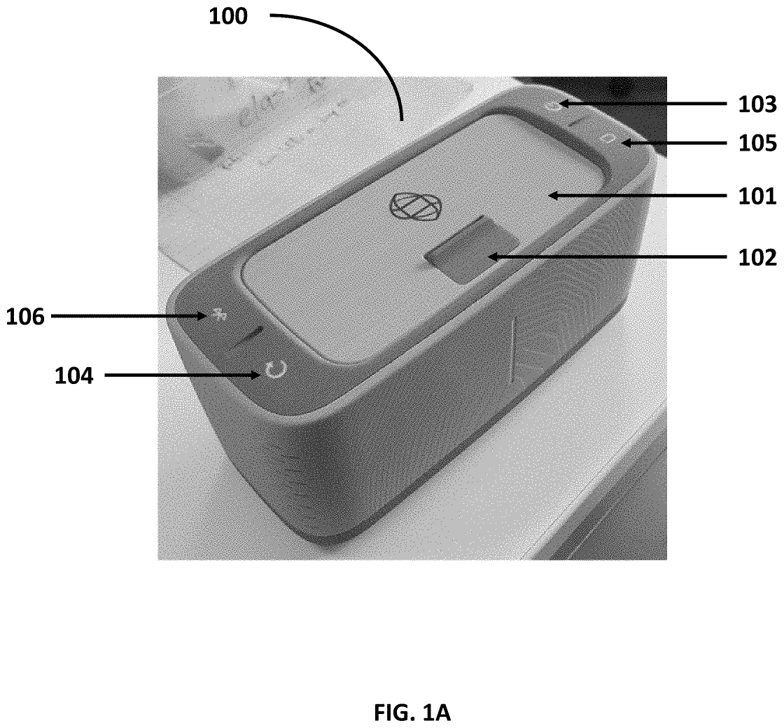

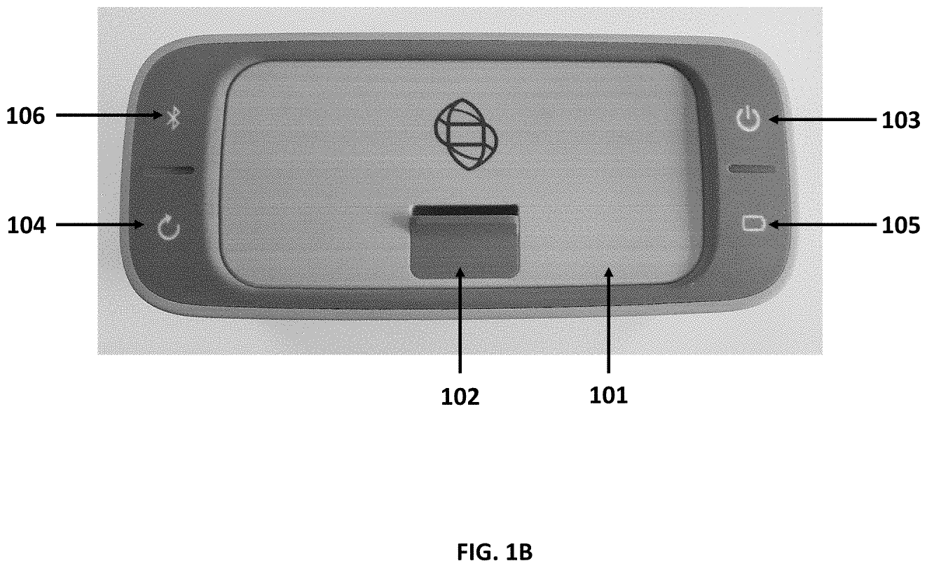

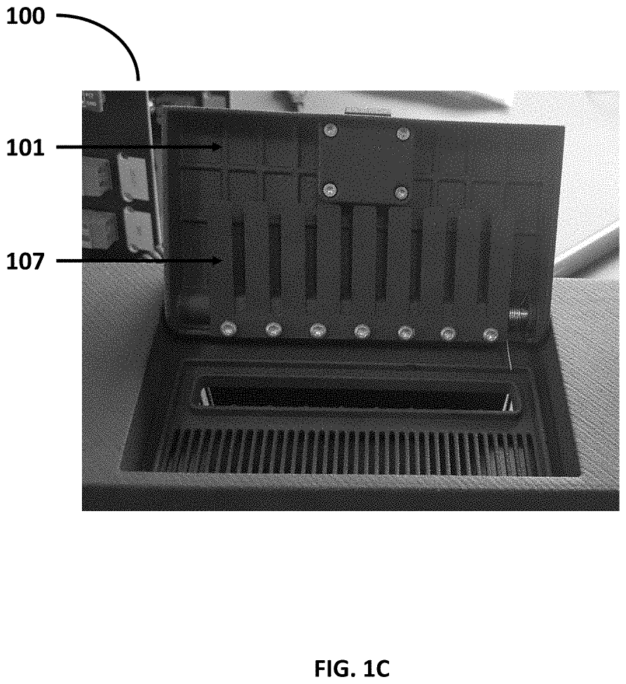

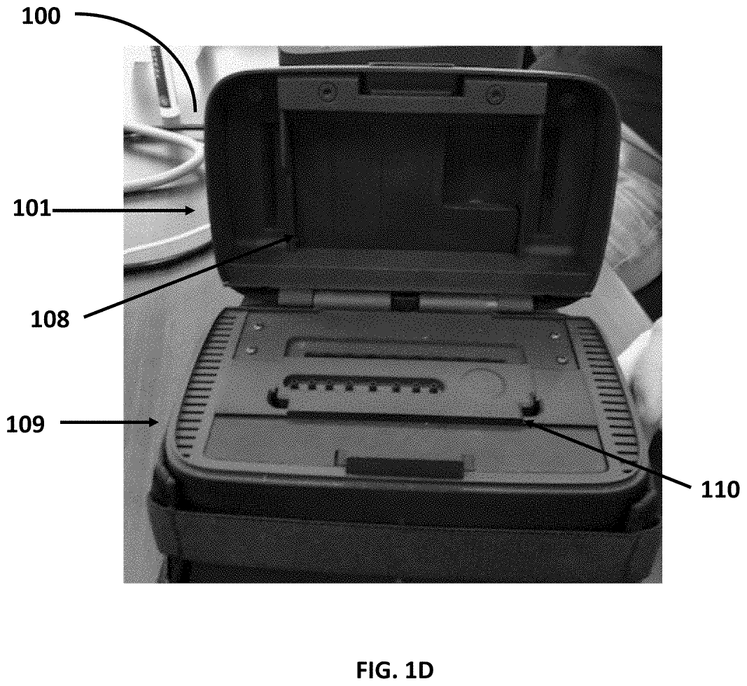

An analytic device of the present disclosure may be used for processing and/or analyzing a sample, such as a biological sample. An analytic device of the present disclosure may be portable. For example, an analytic device may be hand-held. FIGS. 1A-1B show (A) perspective and (B) side views of a housing 100 for a portable analytic device for analyzing a biological sample. A housing may have a lid 101, a securing unit 102 for securing the lid in an open or closed position, and/or buttons or indicators 103-106. Housing 100 may comprise a button 103 for powering on/off the device. Housing 100 may comprise a button 104 for restarting the device. Housing 100 may comprise an indicator 105 for notifying a user that the battery is low and/or an indicator 106 that a wireless connection (e.g., a Bluetooth or Near Field Communication connection) has been established between the analytic device and a mobile electronic device. In some cases, the analytic device described herein can be an analysis unit within a system for sample processing and analyzing. The analytic unit can be within a same housing of a sample preparation unit (e.g., the sample preparation device described herein).

An analytic device may comprise at least one button capable of, upon actuation, affecting the operability of the analytic device (e.g., powering on/off the device or connecting the analytic device to other devices). An analytic device may comprise 1, 2, 3, 4, 5, or more buttons. For example, an analytic device may comprise 4 buttons. Each button may correspond to a different function or feature of the analytic device. In some cases, pairs of buttons may correspond to the same function or feature of the analytic device. For example, an analytic device may include a button to increase a value, zoom level, volume, or other characteristic as well as a button to decrease the same value, zoom level, volume, or other characteristic.

A button mechanism may be a physical mechanism. For example, a button may comprise a depressible mechanism, such as button or micro-switch. Alternatively, a button may comprise a slidable or rotatable mechanism. For analytic devices including two or more buttons, each button may be separately selected from the group consisting of depressible mechanisms, slidable mechanisms, and rotatable mechanisms.

A button may comprise a touch-sensitive feature or mechanism. For example, buttons 103 and 104 of FIGS. 1A and 1B may comprise a touch-sensitive feature or mechanism. A touch-sensitive mechanism may be a touch-sensitive virtual mechanism (e.g., a virtual button). Such a virtual mechanism may be virtually depressible, virtually slidable, or virtually rotatable, thereby giving the illusion of a physical button. For example, the analytic device may comprise or be configured to accept a mobile electronic device communicatively coupled with a wireless connection to the analytic device, and the mobile electronic device may comprise one or more virtual buttons. Depression of a virtual button of the mobile electronic device may transmit a signal from the mobile electronic device to the analytic device, thereby affecting, e.g., a thermocycling program or other process, as described herein. A connection between an analytic device and a mobile electronic device may comprise a one-way or two-way wired or wireless connection, such as a WiFi connection, a Bluetooth connection, a Bluetooth LE connection, an ANT+ connection, or a Gazell connection.

An analytic device may comprise one or more buttons disposed anywhere on the external surface of a housing of the analytic device. For example, a button may be located on a front face, a back face, a right side, a left side, a top side, or a bottom side of a housing of an analytic device. A button may be disposed in a location that is unavailable or hidden during operation of an analytic device (e.g., on the bottom side of a housing of the analytic device). In some cases, a panel may be used to cover or hide one or more buttons (e.g., when the analytic device is not in use and/or to prevent accidental actuation of a button).

Actuation or activation of one or more buttons may permit the user to cycle between a plurality of different thermocycling programs. For example, actuation of a button may cause an analytic device to switch from executing a first thermocycling program to a second thermocycling program. In another example, actuation of a button may cause an analytic device to switch from an “off” state to executing a first thermocycling program. Actuation of the button a second time may cause the analytic device to switch from executing a first thermocycling program to an “off” state. It should be appreciated that an “off” state may refer to an idle state (e.g., wherein an analytic device may be on but a thermocycling program is paused, or wherein the analytic device is in a minimal power state) or a powered-down state (e.g., wherein the analytic device is powered off). Actuation of a button may affect a parameter of a thermocycling program. For example, an analytic device may comprise a depressible mechanism, and actuation of the depressible mechanism may cause a thermocycling program to switch from a denaturation step to an annealing step. In another example, an analytic device may comprise a rotatable mechanism, and rotation of the rotatable mechanism may cause a thermocycling temperature to increase. In some cases, actuation of two or more buttons may affect a thermocycling program.

The degree of an input may affect the state of a thermocycling program. Non-limiting examples of a degree of an input that may be varied include a number of inputs (e.g., a number of times a button is actuated and released in succession), a speed of an input (e.g., a speed at which a button is actuated and/or released), a duration of an input (e.g., an amount of time that a button is actuated), a force exerted for the input (e.g., a force with which a button is actuated), and a direction of an input. An input may comprise actuation of a button. In one example, an analytic device may comprise a depressible mechanism, and brief (e.g., less than half of one second) depression and subsequent release of the depressible mechanism may pause a thermocycling program. In another example, a paused thermocycling program may be resumed by depressing a depressible mechanism for, e.g., 1-2 seconds.

An analytic device may be configured to accept one or more containers including a sample. For example, an analytic device may be configured to accept one or more assay tubes. An assay tube for use with an analytic device of the present disclosure may have any useful size and shape and comprise any useful material. For example, an assay tube may comprise a plastic, a polymer, or glass. An analytic device may be configured to accept an assay tube having a cross section that is substantially cylindrical, substantially rectangular, or has any other shape (e.g., a star shape). An analytic device may be configured to accept an assay tube having a mechanical key element such as a groove or protrusion disposed at one end of the assay tube or along a dimension of the assay tube to facilitate placement of the assay tube in the analytic device. For example, an assay tube may comprise a substantially rectangular protrusion along its length and the analytic device may comprise a corresponding indentation configured to accept the assay tube in a particular orientation. An analytic device may be configured to accept an assay tube having a cap or lid. Alternatively, an analytic device may comprise a component configured to cover an opening of an assay tube when the assay tube is placed in the analytic device. An analytic device may be configured to accept one or more assay tubes. For example, an analytic device may be configured to accept 1, 2, 3, 4, 5, 6, 7, 8, 9, or more assay tubes.

A device described herein can have a surface or support to receive a reagent tube or a cartridge. The cartridge can be a reagent cartridge. The surface or support can be a recessed surface or support. The surface can be a protruded surface or support. The surface can be a chamber. The cartridge can be loaded onto the surface or support. Upon loading the cartridge onto the surface or support, a lid can be closed to click the cartridge in place.

As shown in FIG. 1C, an inner surface of a lid 101 of housing 100 of the analytic device may comprise one or more cantilevers 107 capable of applying pressure to one or more assay tubes seated in a heating block of the analytic device. A cantilever may be useful for securing an assay tube containing a sample against the heating block, thereby increasing the efficiency of energy transfer between the heating block and the assay tube. A cantilever may be heated (e.g., at a temperature equal to the temperature of the heating block) to effect heating of a portion of the assay tube not in contact with the heating block. A cantilever may be heated to any temperature, and the temperature of the cantilever may change throughout a thermal cycle. For example, the temperature of a cantilever may be coordinated (e.g., to be the same as) the temperature of the heating block throughout a thermal cycle. As shown in FIG. 1D, an inner surface of a lid 101 of housing 100 of the analytic device may comprise a recessed surface 108 to receive or accommodate a cartridge inserted into the device. An inner surface of the body 109 of housing 100 of the analytic device may comprise a protruded surface 110 to receive a cartridge inserted into the device.

An analytic device may be portable. For example, an analytic device including a housing may be able to be easily carried or moved. A size, weight and/or shape of the housing and/or other components may affect the portability of the analytic device. A volume of a housing of an analytic device may be less than about 100,000 cubic centimeters, less than about 50,000 cubic centimeters, less than about 10,000 cubic centimeters, less than about 9,000 cubic centimeters, less than about 8,000 cubic centimeters, less than about 7,000 cubic centimeters, less than about 6,000 cubic centimeters, less than about 5,000 cubic centimeters, less than about 4,500 cubic centimeters, less than about 4,000 cubic centimeters, less than about 3,500 cubic centimeters, less than about 3,000 cubic centimeters, less than about 2,500 cubic centimeters, less than about 2,000 cubic centimeters, less than about 1,500 cubic centimeters, less than about 1,400 cubic centimeters, less than about 1,300 cubic centimeters, less than about 1,200 cubic centimeters, less than about 1,100 cubic centimeters, less than about 1,000 cubic centimeters, less than about 900 cubic centimeters, less than about 800 cubic centimeters, less than about 700 cubic centimeters, less than about 600 cubic centimeters, or less than about 500 cubic centimeters. For example, a volume of a housing of an analytic device may be less than about 1,500 cubic centimeters. A volume of a housing of an analytic device may fall within a range. For example, a volume of a housing of an analytic device may be between about 500 cubic centimeters and about 1,500 cubic centimeters. A dimension of the housing (e.g., length, width or height) may be at most about 50 centimeters, at most about 40 centimeters, at most about 30 centimeters, at most about 25 centimeters, at most about 24 centimeters, at most about 23 centimeters, at most about 22 centimeters, at most about 21 centimeters, at most about 20 centimeters, at most about 19 centimeters, at most about 18 centimeters, at most about 17 centimeters, at most about 16 centimeters, at most about 15 centimeters, at most about 14 centimeters, at most about 13 centimeters, at most about 12 centimeters, at most about 11 centimeters, at most about 10 centimeters, at most about 9 centimeters, at most about 8 centimeters, at most about 7 centimeters, at most about 6 centimeters, or at most about 5 centimeters.

A weight of an analytic device including the housing may be less than about 25 kilograms, less than about 20 kilograms, less than about 15 kilograms, less than about 10 kilograms, less than about 5 kilograms, less than about 4.5 kilograms, less than about 4 kilograms, less than about 3.5 kilograms, less than about 3 kilograms, less than about 2.5 kilograms, less than about 2.4 kilograms, less than about 2.3 kilograms, less than about 2.2 kilograms, less than about 2.1 kilograms, less than about 2 kilograms, less than about 1.9 kilograms, less than about 1.8 kilograms, less than about 1.7 kilograms, less than about 1.6 kilograms, less than about 1.5 kilograms, less than about 1.4 kilograms, less than about 1.3 kilograms, less than about 1.2 kilograms, less than about 1.1 kilograms, less than about 1 kilogram, less than about 0.9 kilograms, less than about 0.8 kilograms, less than about 0.7 kilograms, less than about 0.6 kilograms, less than about 0.5 kilograms, less than about 0.4 kilograms, less than about 0.3 kilograms, less than about 0.2 kilograms, or less than about 0.1 kilograms. For example, a volume of a housing of an analytic device may be less than about 1.5 kilograms. A weight of an analytic device including a housing may fall within a range of weights. For example, a weight of an analytic device including a housing may be between about 0.5 kilograms and about 1.5 kilograms.

A shape of a housing of an analytic device may also contribute to the portability of the analytic device. At least one dimension of a housing (e.g., length, width or height), may be sufficiently small such that the housing may be easily grasped by the human hand. An analytic device may have an ergonomically shaped housing of a size that enables a user to hold the analytic device with one or two hands. The housing may comprise a gripping region, e.g., a portion of the housing that is gripped by the user when the user holds the analytic device. A gripping region of a housing may be shaped to conform to the fingers of the user, thereby allowing the user to maintain a secure grip on the housing. A front surface of a housing of an analytic device may be narrower in a middle section associated with a gripping region than at a top or bottom section of the front surface. The narrower section may be conveniently and securely gripped by the user, while the relatively wider top section may include a display device or a component thereof, such as a screen. A housing may comprise a retractable handle that may be ergonomically shaped. A housing of an analytic device may feature rounded corners and/or edges (e.g., where perpendicular surfaces meet) such that when a user holds the analytic device, the user's hand may be in contact with rounded corners rather than sharp corners.

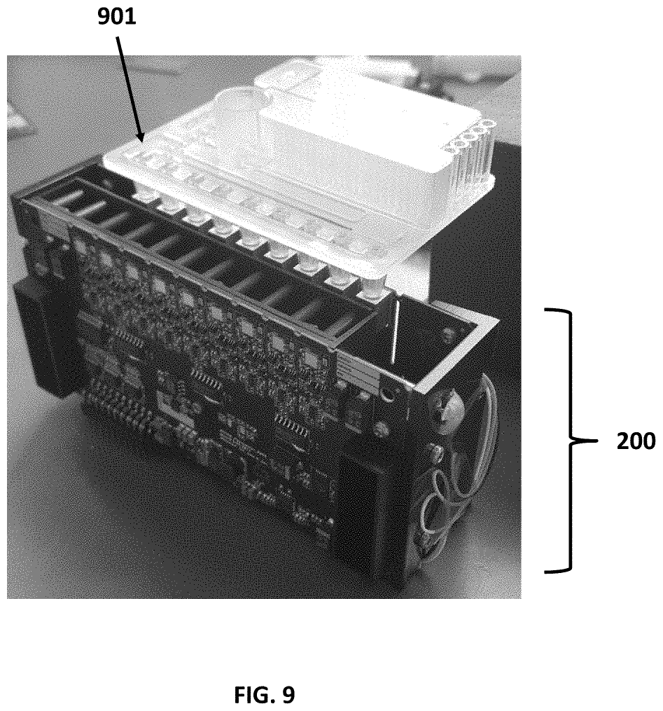

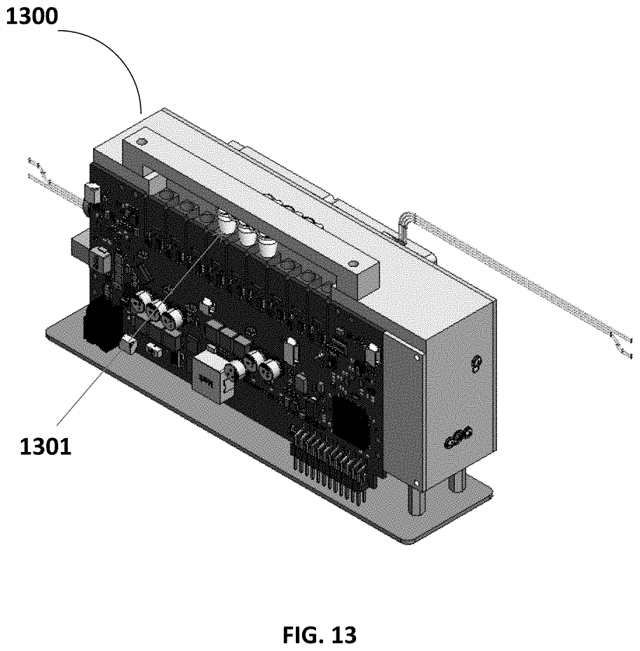

FIG. 9 shows an example portable device having a sample cartridge 901 inserted into the device for sample analysis. A perspective view of an internal mechanism 200 is shown. FIG. 13 shows another example of the portable device 1300 having sample tubes 1301 inserted into the device for sample analysis.

### Thermocycling

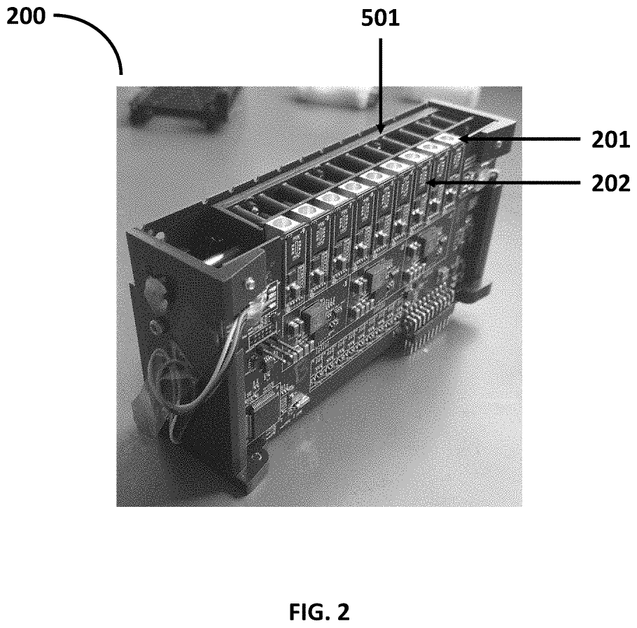

An analytic device may be configured to heat or cool a sample within an assay tube. As shown in FIG. 2, an analytic device 200 may comprise one or more heating blocks 201 within which an assay tube containing a sample is placed. The analytic device may be configured to raise or lower the temperature of the heating block using a heater 202 (e.g., a resistive heater) in discrete steps.

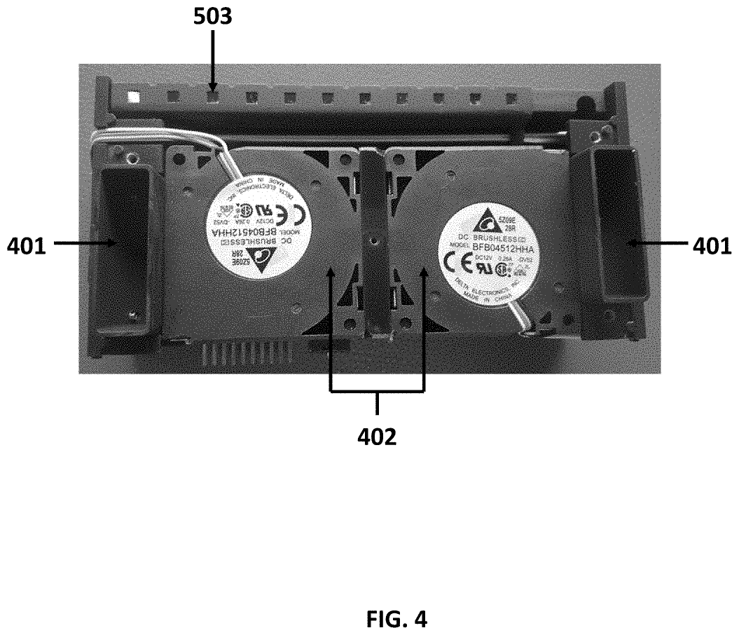

In some cases, the heating block can convert electrical energy into heat through the process of resistive or Joule heating. The heating block can be a resistive heater. Heated blocks can have power resister (e.g., thermister), thermal epoxy to bring in thermal communication with sample chambers. The heating blocks may be level and uniform. Cooling of the heating block can be achieved or controlled through a fan. For example, FIG. 4 shows a rear view of an internal mechanism for a portable analytic device with a circuit board removed, thereby exposing fans 402 of the internal mechanism.

In some cases, the heating block can be a Peltier heater. Heating and cooling can be achieved or controlled through a Peltier controller. In some other cases, the heating block may not be a Peltier heater or the heating block may not be controlled by a Peltier controller.

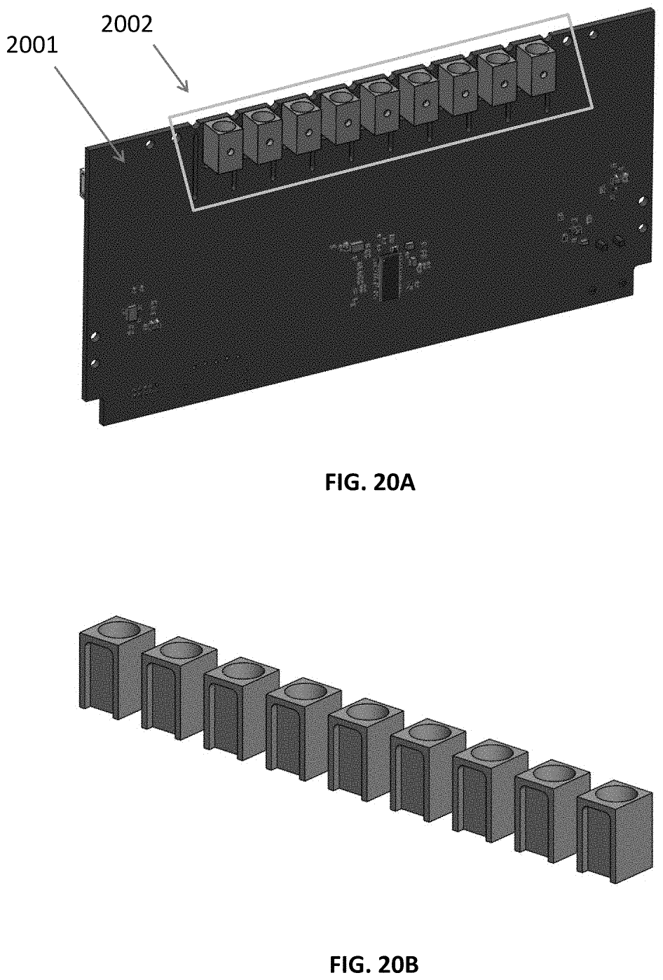

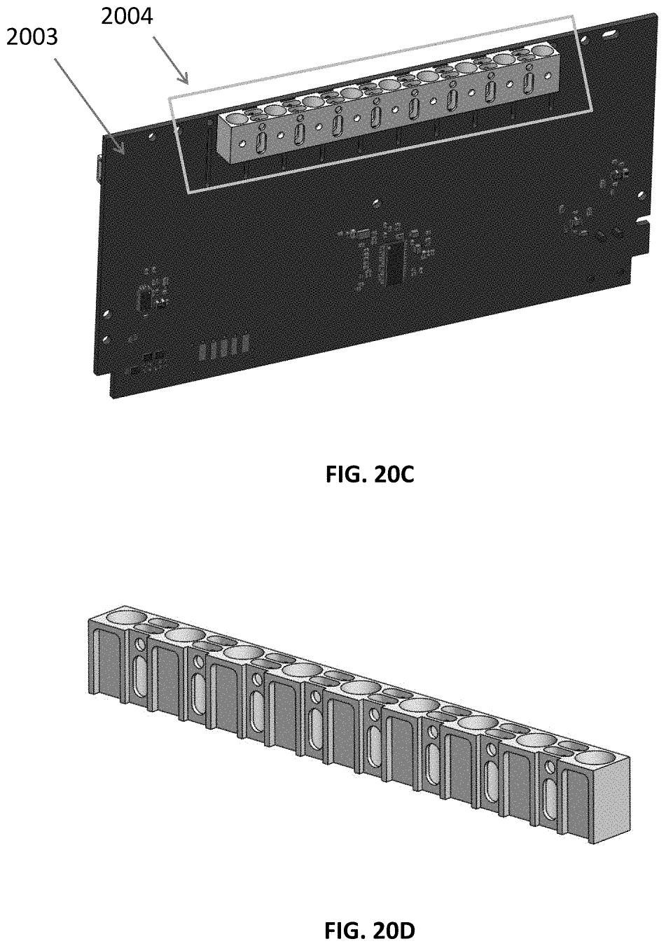

The analytic device may comprise one or more heating blocks. In some cases, two or more heating blocks are independent, unconnected or separated from each other (e.g., single block model/configuration as shown in FIG. 20A and FIG. 20B). For example, a heating block and an adjacent heating block may not be connected such that there is a gap region between two adjacent heating blocks. The two or more heating blocks may be individually installed into the device. FIG. 20A shows plurality of individual heating blocks 2002 installed on a circuit board 2001 of an analytic device.

In some cases, two or more individual heating blocks are connected. In some cases, two or more heating blocks are connected to form a strip of heating blocks (e.g., monolithic block model/configuration as shown in FIG. 20C and FIG. 20D). Multiple individual heating blocks may be brought in contact with one another to yield a single heating unit (e.g., a monolithic heating block). The individual heating blocks may be formed of the same material or different materials. Adjacent heating blocks may be in direct contact with one another. Adjacent heating blocks may be in contact with one another through a thermally conductive material (e.g., a thermally conductive epoxy).

A monolithic heating block can comprise a plurality of heating subunits (e.g., each subunit can be a functional equivalent to an individual heating block). The heating subunits may be formed of the same material (e.g., by removing material from a monolithic object to generate recesses). For example, FIG. 20C shows a monolithic heating block 2004 having a plurality of heating subunits installed on a circuit board 2003 of an analytic device.

A heating block can be connected to an adjacent heating block or blocks such that there may not be any gap regions between the heating block and the adjacent heating block or blocks. The gap region between two adjacent heating blocks may be filled with solid materials to connect the two adjacent heating blocks. In some cases, the entire volume of the gap region is filled with the solid material. In some cases, a portion of the gap region is filled with the solid material. For example, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more of the entire volume of the gap region can be filed with the solid material to connect the adjacent heating blocks. The solid material in the gap region may be molded into a variety of configurations. For example, the solid material in the gap region may form one or more bridges connecting adjacent heating blocks. For example, the solid material in the gap region may have a hollow structure. The solid material in the gap region may comprise one or more holes. The solid material can be a variety of materials. The solid material may be the same material or different material from the material used to make the heating blocks as described herein. Non-limiting examples of materials that may be used as the solid material in the gap regions include aluminum, concrete, glass, quartz, steel, iron, nickel, zinc, copper, brass, silver, tin, gold, carbon, and any combination thereof (e.g., a zinc alloy such as Zamak).

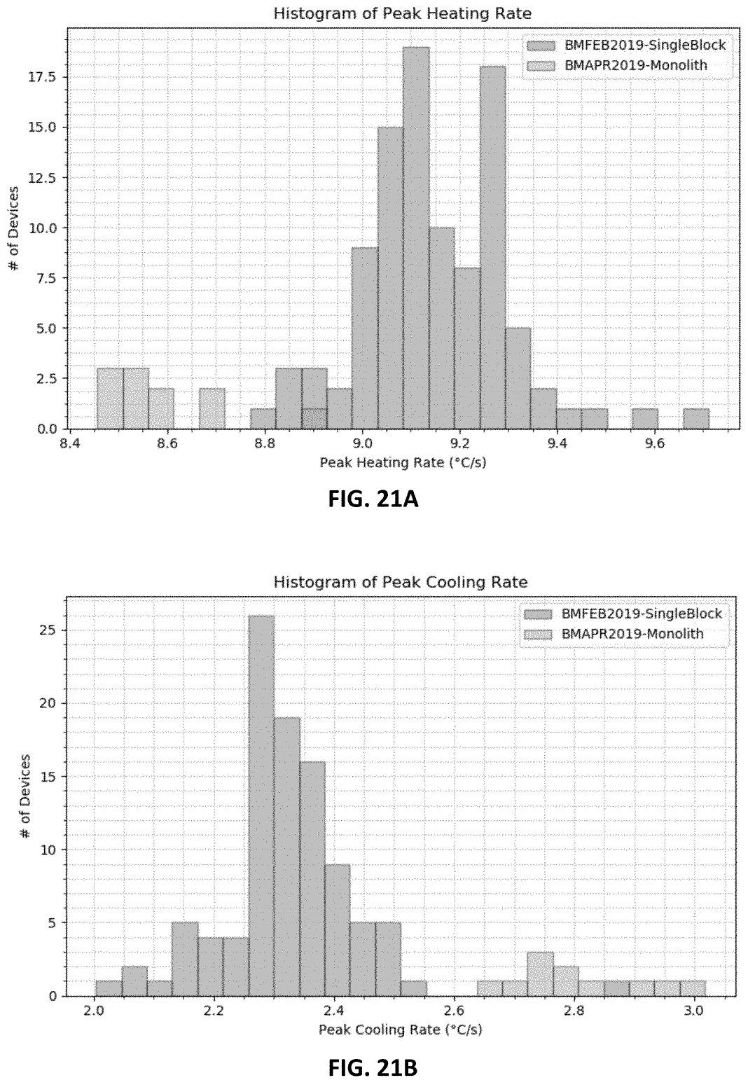

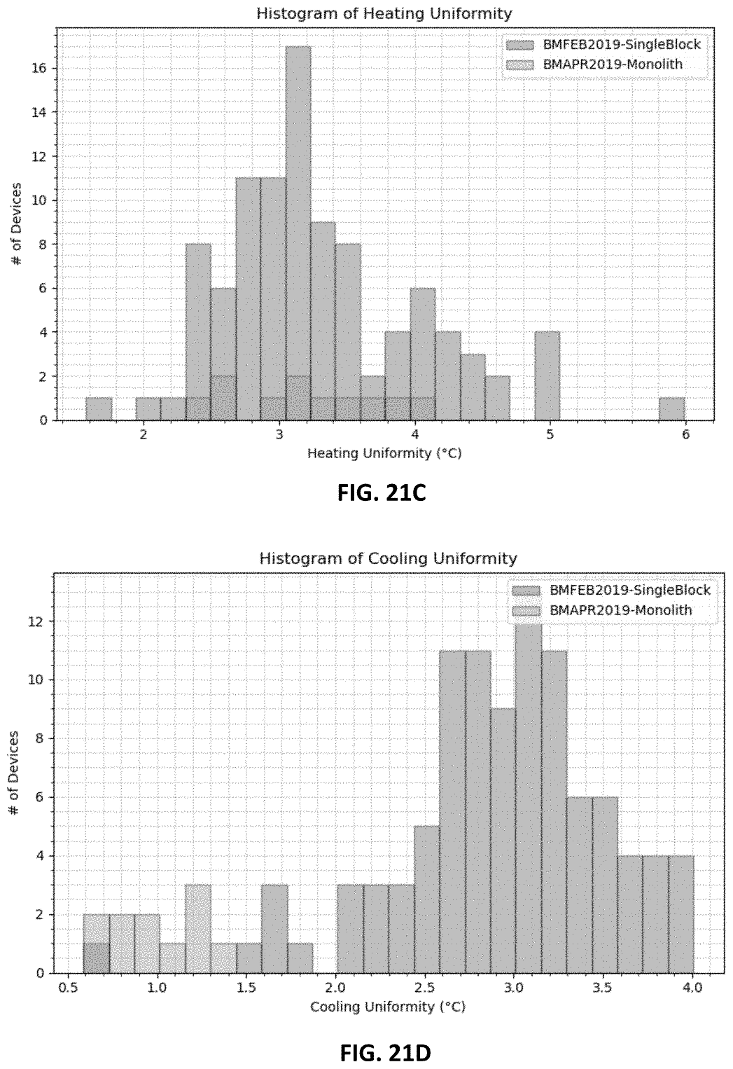

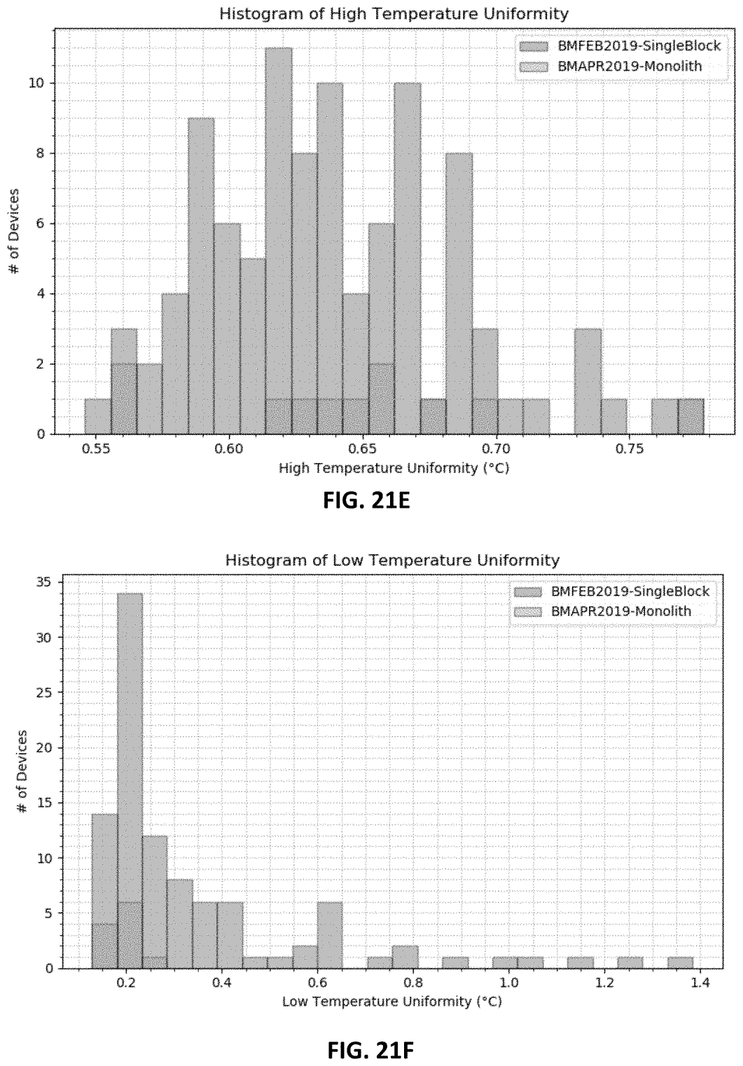

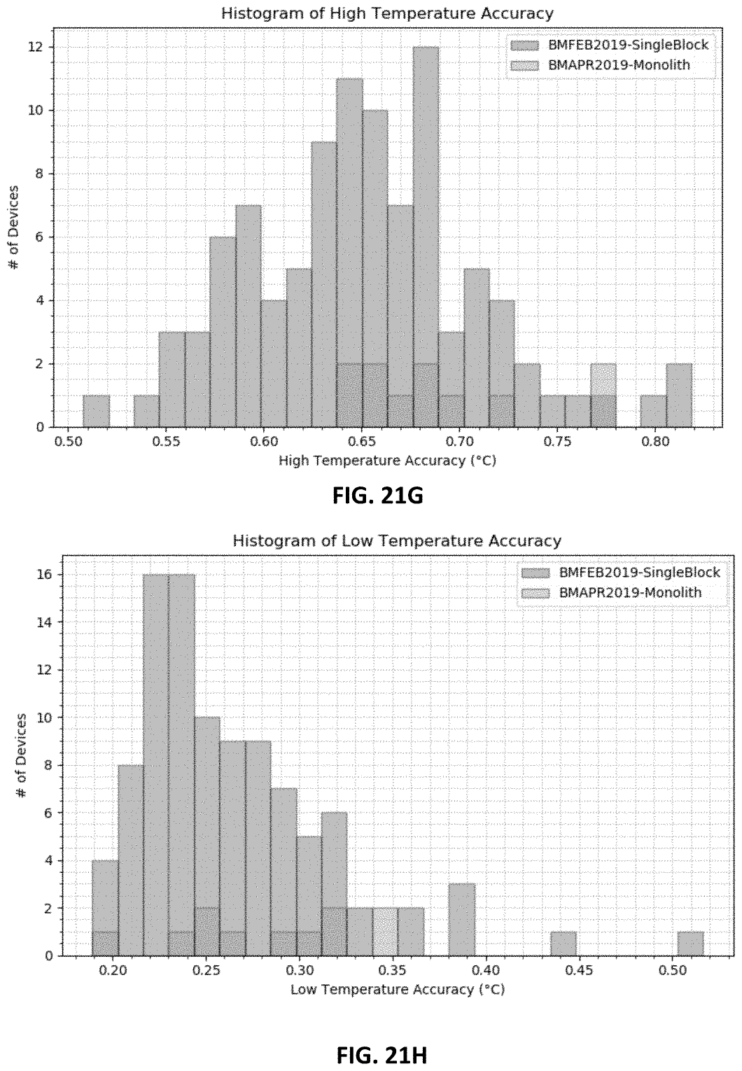

In some cases, devices having individual or unconnected blocks (e.g., single blocks, as shown in FIG. 20A) may be heated faster (e.g., at a greater heating rate) than devices having connected heating blocks (e.g., a monolithic block, as shown in FIG. 20C). FIG. 21A shows an example histogram of peak heating rate of single block devices and monolithic block devices. In this example, ninety-nine single block devices and eleven monolithic block devices were tested. The devices were cured with Masterbond Supreme 18TC Epoxy. The temperatures were cycled between 50° C. and 100° C. In comparison, single block devices were heated faster (or had a greater heating rate) than monolithic block devices.

A heating rate of a monolithic block may be less than a heating rate of unconnected blocks. However, a cooling rate of the monolithic block may be greater than the cooling rate of the unconnected blocks. For example, the cooling rate of the monolithic block may be at least 1%, 5%, 10%, 20%, 30%, 40%, 50%, 60%, 70%, 80%, 90%, or more greater than the cooling rate of the unconnected blocks.

Two or more heating blocks can be connected to facilitate more efficient heat exchange during cooling than two or more unconnected heating blocks. For example, two or more heating blocks, when in the unconnected configuration, may trap heat in the center of each heating block, resulting in longer cooling time to reach a given temperature. When the two or more heating blocks are connected, it may take less time to achieve a given temperature during cooling. FIG. 21B shows an example histogram of peak cooling rate of single block devices and monolithic block devices. In this example, ninety-nine single block devices and eleven monolithic block devices were tested. The devices were cured with Masterbond Supreme 18TC Epoxy. The temperatures were cycled between 50° C. and 100° C. In comparison, monolithic block devices were cooled faster than single block devices.

The devices having individual blocks and the devices having connected blocks may have comparable heating uniformity. Uniformity can be calculated as the hottest block minus the coolest block for all points in time. FIG. 21C shows an example histogram of heating uniformity of single block devices and monolithic block devices. In this example, ninety-nine single block devices and eleven monolithic block devices were tested. The devices were cured with Masterbond Supreme 18TC Epoxy. The temperatures were cycled between 50° C. and 100° C. Single block devices had similar performance on heating uniformity to the monolithic block devices. The devices having individual blocks and the devices having connected blocks may not have comparable cooling uniformity. FIG. 21D shows an example histogram of cooling uniformity of single block devices and monolithic block devices. In this example, ninety-nine single block devices and eleven monolithic block devices were tested. The devices were cured with Masterbond Supreme 18TC Epoxy. The temperatures were cycled between 50° C. and 100° C. Monolithic block devices had more uniform temperatures during cooling compared with the single block devices. FIG. 21E shows an example histogram of high temperature (˜100° C.) uniformity of single block devices and monolithic block devices. In this example, ninety-nine single block devices and eleven monolithic block devices were tested. The devices were cured with Masterbond Supreme 18TC Epoxy. The heating blocks were heated to a temperature of 100° C. Single block devices had similar performance on high temperature uniformity to the monolithic block devices. FIG. 21F shows an example histogram of low temperature (˜50° C.) uniformity of single block devices and monolithic block devices. In this example, ninety-nine single block devices and eleven monolithic block devices were tested. The devices were cured with Masterbond Supreme 18TC Epoxy. The heating blocks were heated to a temperature of 50° C. Single block devices had similar performance on low temperature uniformity to the monolithic block devices.

The devices having individual blocks and the devices having connected blocks may or may not have comparable accuracy. Accuracy can be calculated by the difference between average reaction block temperature and set temperature at a defined moment in time. FIG. 21G shows an example histogram of accuracy at high temperature of single block devices and monolithic block devices. In this example, ninety-nine single block devices and eleven monolithic block devices were tested. Monolithic block devices were slightly less accurate at the high temperature compared with single block devices. FIG. 2111 shows an example histogram of accuracy at low temperature of single block devices and monolithic block devices. In this example, ninety-nine single block devices and eleven monolithic block devices were tested. Monolithic block devices had comparable accuracy at the low temperature compared with single block devices.

The device described herein may or may not comprise a heated lid.

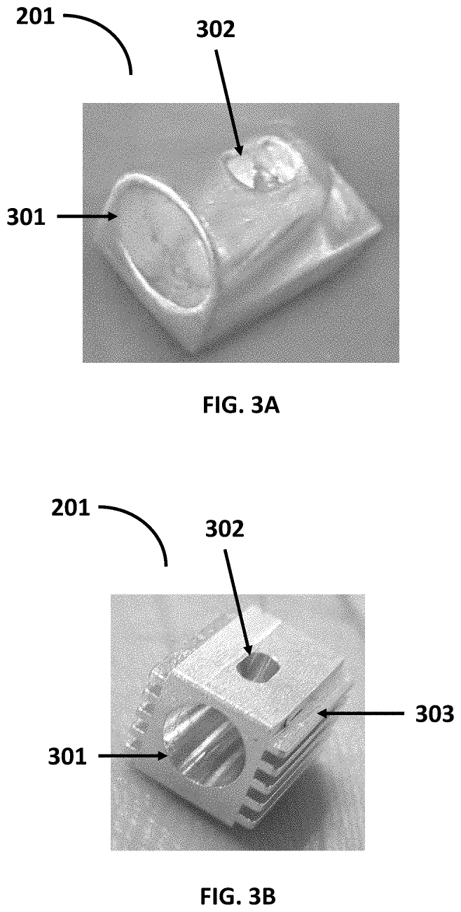

A heating block 201 may comprise any useful material. Non-limiting examples of materials that may be used to construct a heating block include aluminum, concrete, glass, quartz, steel, iron, nickel, zinc, copper, brass, silver, tin, gold, carbon, and any combination thereof (e.g., a zinc alloy such as Zamak). For example, a heating block may be constructed using silver, as shown in FIG. 3A. In another example, a heating block may be constructed using aluminum, as shown in FIG. 3B. The heating block may include a first opening 301 for accepting a vial containing or configured to contain a sample (e.g., biological sample), and a second opening 302 configured to be in optical communication with a detector or an optical source (e.g., for excitation). The heating block may include a third opening (not shown) configured to be in optical communication with a detector or an optical source. For example, the second opening 302 may be in optical communication with a detector and the third opening (not shown) may be in optical communication with an optical source for excitation. The heating block may comprise one or more fins 303.

A heating block may be formed of an alloy. For example, a heating block may be constructed using steel. It is contemplated that constructing the heating block using a material compatible with the process of die casting, (e.g., a material that that may be used in the die cast construction of a heating block) can allow for the heating blocks to be manufactured at a larger scale (e.g., at a higher volume in a shorter period of time, and/or at a reduced cost per unit). In some embodiments, a heating block can be constructed using a combination of materials. For example, a heating block can be constructed using aluminum and subsequently coated with nickel. In another example, a heating block can be constructed using zinc, and coated with silver. Coating the heating block can be advantageous for several reasons. For example, coating a heating block (e.g., with nickel) can allow the heating block to be soldered to a printed circuit board (PCB), as opposed to using thermal epoxy. Soldering the heating block to the PCB can allow an analytic device to be manufactured with a removable heating block (e.g., in the case of damage), whereas the use of a thermal epoxy can permanently affix the heating block to the PCB. It is contemplated that the choice of the material used to produce the heating block may affect the number of thermal cycles that the analytic device is capable of undergoing using a power supply (e.g., a self-contained power supply, such as a battery). In particular, the higher the specific heat capacity of the material, the more energy may be used to raise the temperature of the material. Accordingly, a heating block can be constructed using a material with a specific heat capacity (e.g., at 25° C., as measured in Joules per gram per ° C.; J/g° C.) of less than about 2 J/g° C., less than about 1.5 J/g° C., less than about 1 J/g° C., less than about 0.9 J/g° C., less than about 0.8 J/g° C., less than about 0.7 J/g° C., less than about 0.6 J/g° C., less than about 0.5 J/g° C., less than about 0.45 J/g° C., less than about 0.4 J/g° C., less than about 0.35 J/g° C., less than about 0.3 J/g° C., less than about 0.25 J/g° C., less than about 0.2 J/g° C., less than about 0.15 J/g° C., less than about 0.1 J/g° C., less than about 0.05 J/g° C., or less than about 0.01 J/g° C. For example, a heating block can be constructed using a material having a specific heat capacity of less than about 1 J/g° C. at 25° C.

Additionally, the lower the thermal conductivity of a material, the more energy may be used to raise the temperature of the material. Accordingly, a heating block can be constructed using a material with a thermal conductivity (e.g., as measured in Watt per meter per Kelvin; W/mK) of at least about 500 W/mK, at least about 400 W/mK, at least about 300 W/mK, at least about 200 W/mK, at least about 175 W/mK, at least about 150 W/mK, at least about 125 W/mK, at least about 100 W/mK, at least about 75 W/mK, at least about 50 W/mK, at least about 25 W/mK, or at least about 10 W/mK. For example, a heating block can be constructed using a material having a thermal conductivity of at least about 75 W/mK. In another example, a heating block can be constructed using a material having a thermal conductivity of at least about 400 W/mK.

A heating block may also comprise one or more fins 303 to increase a surface area of the heating block and provide better heat dissipation from the heating block. It is also contemplated that the volume of the material used to form a heating block may affect the number of thermal cycles that the analytic device is capable of undergoing using a power supply (e.g., a self-contained power supply, such as a battery). In particular, the greater the volume of the material used to construct the heating block, the more energy may be used to raise the temperature of the heating block. Accordingly, a volume of a material used to construct a heating block may be less than about 20 cubic centimeters, less than about 15 cubic centimeters, less than about 10 cubic centimeters, less than about 9 cubic centimeters, less than about 8 cubic centimeters, less than about 7 cubic centimeters, less than about 6 cubic centimeters, less than about 5 cubic centimeters, less than about 4 cubic centimeters, less than about 3 cubic centimeters, less than about 2 cubic centimeters, less than about 1 cubic centimeters, less than about 0.9 cubic centimeters, less than about 0.8 cubic centimeters, less than about 0.7 cubic centimeters, less than about 0.6 cubic centimeters, less than about 0.5 cubic centimeters, less than about 0.4 cubic centimeters, less than about 0.3 cubic centimeters, less than about 0.2 cubic centimeters, or less than about 0.1 cubic centimeters. For example, a volume of a material used to construct a heating block may be less than about 0.5 cubic centimeters.

As described above, the material and/or volume of material used to construct the heating may be selected based on minimizing the energy used to heat or cool the block. Accordingly, an analytic device of the present disclosure may provide more energy to perform a greater number of thermal cycles, as compared to a device that uses a larger heating block, or a heating block constructed using a material with a higher specific heat capacity. An analytic device of the present disclosure may perform any number of thermal cycles. An analytic device may perform a given number of thermal cycles on a single charge of a power supply (e.g., a self-contained power supply, such as a battery). An analytic device of the present disclosure may perform at least about 1 thermal cycle, at least about 2 thermal cycles, at least about 3 thermal cycles, at least about 4 thermal cycles, at least about 5 thermal cycles, at least about 6 thermal cycles, at least about 7 thermal cycles at least about 8 thermal cycles, at least about 9 thermal cycles, at least about 10 thermal cycles, at least about 11 thermal cycles, at least about 12 thermal cycle, at least about 13 thermal cycles, at least about 14 thermal cycles, at least about 15 thermal cycles, at least about 16 thermal cycles, at least about 17 thermal cycles, at least about 18 thermal cycles at least about 19 thermal cycles, at least about 20 thermal cycles, at least about 25 thermal cycles, at least about 30 thermal cycles, at least about 35 thermal cycle, at least about 40 thermal cycles, at least about 45 thermal cycles, at least about 50 thermal cycles, or at least about 100 thermal cycles. An analytic device of the present disclosure may perform about 1 to about 10 thermal cycles, about 5 to about 15 thermal cycles, about 10 to about 20 thermal cycles, or about 15 to about 25 thermal cycles.

An analytic device of the present disclosure may be configured to perform an amplification reaction such as polymerase chain reaction (PCR) (e.g., by cycling the temperature of a sample in an assay tube). Performing PCR may involve making a series of repeated temperature changes (e.g., thermal cycles) with each series (e.g., cycle) including two or three discrete temperature steps. Thermal cycling may be preceded by a single temperature step at a higher temperature (e.g., >90° C.). Temperatures used and the length of time they are applied in each cycle may vary based on, for example, the enzyme used for deoxyribonucleic acid (DNA) synthesis, the concentration of bivalent ions and nucleotides (dNTPs) in the reaction, and the melting temperature (Tm) of one or more primers. The individual steps of an amplification reaction such as PCR may comprise initialization, denaturation, annealing, and/or extension/elongation. Initialization may be used for DNA polymerases that can be activated by heat (e.g., “hot start” PCR). Initialization may comprise heating a sample (e.g., a sample in an assay tube) to a high temperature (e.g., 94-96° C. [201-205° F.) or 98° C. [208° F], if thermostable polymerases are used), which may be maintained for about 1-10 minutes. Denaturation may comprise heating (e.g., to 94-98° C. [201-208° F.]) a sample (e.g., a sample in an assay tube) for a given time such as between about 5 seconds and 5 minutes. This may result in DNA melting, or denaturation, of a double-stranded DNA template by breaking hydrogen bonds between complementary bases, yielding two single-stranded nucleic acid molecules (e.g., templates). Annealing may comprise lowering the temperature of a sample (e.g., a sample in an assay tube) to, e.g., 50-65° C. (122-149° F.) for a given time, such as between about 5 seconds and 5 minutes, thereby allowing annealing of one or more primers to each of the single-stranded nucleic acid templates. At least two different primers may be included in the reaction mixture, including one for each of the two single-stranded nucleic acid templates containing a target region. The primers may be single-stranded nucleic acid molecules themselves. Conditions suitable for effective extension/elongation may depend on the DNA polymerase used. Extension/elongation comprises synthesizing a new DNA strand complementary to a single-stranded nucleic acid template by adding, in the presence of a DNA polymerase, free dNTPs from a reaction mixture that are complementary to the template in the 5′-to-3′ direction and condensing the 5′-phosphate group of the dNTPs with the 3′-hydroxy group at the end of the nascent (elongating) DNA strand. The time for extension/elongation may depend on the DNA polymerase used and/or on the length of the DNA target region to amplify.

Denaturation, annealing, and extension/elongation may constitute a single thermal cycle. Multiple cycles may be used to amplify a DNA target to a detectable level.

The temperature of a heating block may be regulated in any useful way. Thermal energy may be provided to or removed from a sample (e.g., a sample in an assay tube) by heating or cooling, respectively, the heating block. A temperature of a heating block may be controlled (e.g., increased or decreased) using a heating unit (e.g., comprising a resistive, ohmic heater, or flexible heater) and/or a cooling unit (e.g., comprising a thermoelectric cooler or a fan). Temperature monitoring may be necessary for thermocycling applications. Accordingly, a heating or cooling unit may also comprise one or more thermistors and/or temperature transducers to monitor and/or provide feedback to a heating or cooling unit to regulate the temperature of a heating block. A heating or cooling unit may be disposed adjacent to a heating block (e.g., on a surface of a heating block). Alternatively, a heating or cooling unit may be disposed within a recess along a surface of a heating block. A cooling unit may comprise a fan disposed away (e.g., not in direct contact with) a heating block. A fan may be used to apply a positive or negative pressure to a volume adjacent to a heating block, thereby evacuating the area surrounding the heating block. By evacuating the area surrounding the heating block, which may comprise air having radiant heat energy from the heating block, the temperature of the heating block may be reduced. A fan may be used to generate a vacuum to evacuate radiant heat surrounding the heating block. Alternatively, a fan may be used to generate positive pressure to exhaust or force radiant heat surrounding the heating block (e.g., a fluid comprising heat from the heating block) out of the analytic device. As shown in FIG. 4A, radiant heat surrounding the heating block may be removed from the analytic device through one or more vents 401 disposed on the analytic device. One or more fans 402 may be fluidly connected to the space surrounding the heating block and one or more vents. An analytic device may comprise any number of fans. For example, an analytic device may comprise 1, 2, 3, 4, 5, or more fans. An analytic device may comprise one fan for each heating block. Carriage

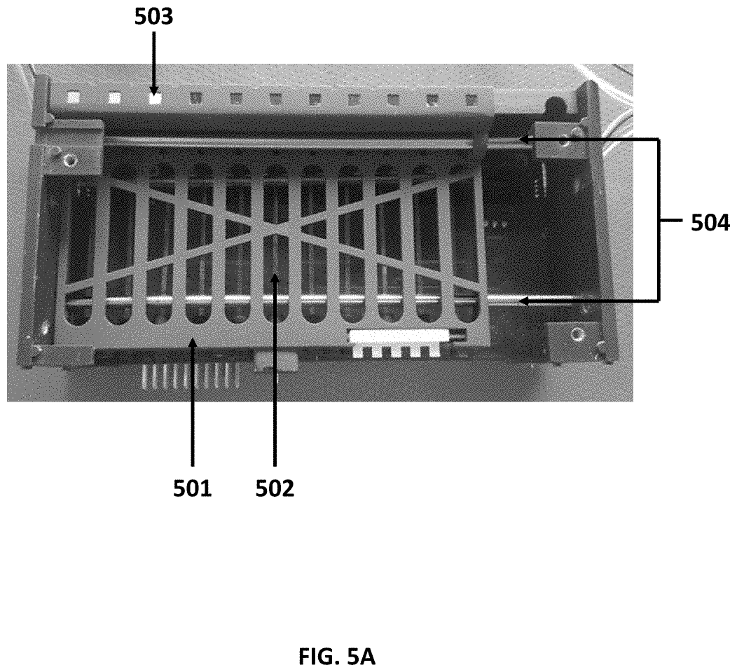

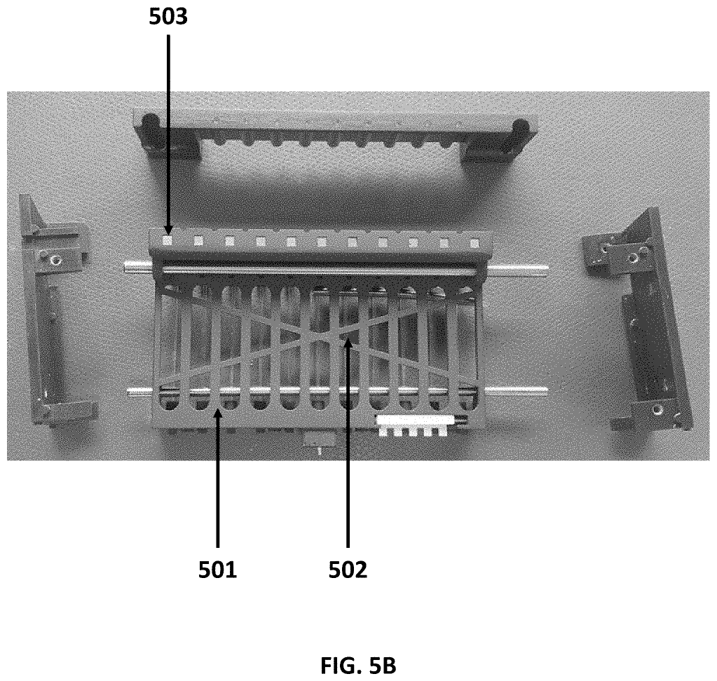

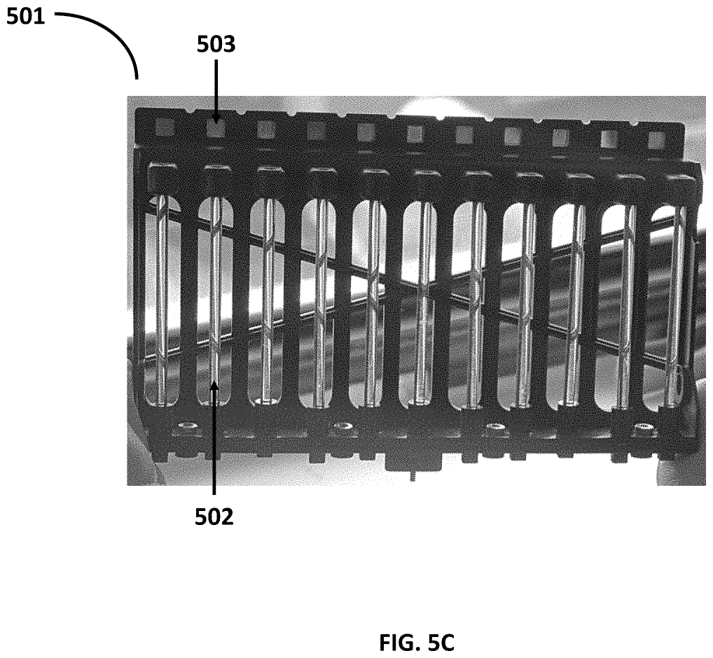

An analytic device may comprise a carriage. A carriage may be used to hold in place or shift one or more optical components (e.g., an optical filter such as an emission filter or an excitation filter, a light path, and/or a light source) to align with a specified assay tube. As shown in FIG. 5A, a carriage 501 may comprise various optical components, such as an excitation filter (not shown), a light path 502 (e.g., a light pipe) to communicate filtered excitation energy to a sample (e.g., a sample in an assay tube), and an emission filter 503 to filter emission energy prior to detection by a detector. FIG. 5B shows a deconstructed view of the carriage mechanism shown in FIG. 5A. FIG. 5C shows a front view of a moving carriage 501 of the internal mechanism, the moving carriage having multiple light paths 502. The carriage may be configured to move along one or more paths, grooves, or rails 504. The carriage may be constructed using any useful material. Non-limiting examples of materials that may be used to construct the carriage include polysiloxane, polyphosphazene, low-density polyethylene (ldpe), high-density polyethylene (hdpe), polypropylene (pp), polyvinyl chloride (pvc), polystyrene (ps), nylon, nylon 6, nylon 6,6, teflon (polytetrafluoroethylene), thermoplastic polyurethanes (tpu), polychlorotrifluoroethylene (pctfe), bakelite, kevlar, twaron, mylar, neoprene, nylon, nomex, orlon, rilsan, technora, teflon, ultem, vectran, viton, zylon, polyamides, polycarbonate, polyester, polyethylene, polyvinylidene chloride (pvdc), acrylonitrile butadiene styrene (abs), polyepoxide, polymethyl methacrylate, maleimide, polyetherimide, polylactic acid, furan, silicone, polysulfone, or a metal or metal alloy (e.g., aluminum, brass, copper, iron, and silver). A light path may comprise an open space of a particular geometry and volume. The space may be defined by a container or guide such as a pipe. A light path (e.g., a light pipe) may be constructed using any useful material. Non-limiting examples of materials that may be used to construct a light path (e.g., a light pipe) include glass, silica, fluorozirconate, fluoroaluminate, chalcogenide, plastic, PMMA, polystyrene, silicone resin, and any combination thereof.

A carriage may be a moving carriage. A moving carriage may be used to shift a light path aligning with a first light source and a first assay tube to a second light source and a second assay tube. Similarly, a moving carriage may be used to shift a sample from aligning with a first light path to align with a second light path. An analytic device comprising a moving carriage may provide certain advantages compared to an analytic device comprising, in lieu of a moving carriage, a stationary component. For example, the inclusion of a moving carriage may allow multiple assay tubes to share light paths and associated components such as optical filters (e.g., excitation and emission filters). This may reduce the cost of producing the analytic device (e.g., by requiring fewer optical filters, e.g., excitation and emission filters, which may be costly). The sharing of light paths may also reduce the overall size of the analytic device (e.g., by reducing the number of optical components necessary for analyzing the sample in each assay tube), thereby making the analytic device more portable. A moving carriage may be configured to move from a first or original position to a final position, making one or more stops at specified positions between the original and final positions. The path between the original and final positions may be a linear path and may comprise one or more grooves, tracks, or rails along which a moving carriage may travel. The path between the original and final positions may comprise one or more specified positions at which the moving carriage may stop (e.g., via a manual or automated control, as described herein). The one or more specified positions may correspond to the positions of one or more assay tubes or seats or housings therefor in an analytic device. A specified position may comprise a mechanical component such as a key to facilitate positioning of the moving carriage in the specified position (e.g., beneath an assay tube). Movement of a moving carriage may be achieved using a variety of methods. For example, an electric motor may be used to move the carriage from a first position to a second position. A motor having a cam may be used to move the carriage via a belt coupled to the carriage and the cam. Movement of a moving carriage may be achieved using a magnetic levitation system. For example, a carriage may be slidably disposed on or in one or more electrified rails or grooves, and a magnetic force generated within a rail or groove may be used to move the carriage. A spring may be used to return a moving carriage to its original position, e.g., after it has moved from its original position to a final position, such as the end of a rail, track, or groove. It is contemplated that constructing the moving carriage using lighter weight materials may reduce the energy used to move the carriage, thereby increasing the amount of energy available for heating and/or cooling the sample and/or other processes.

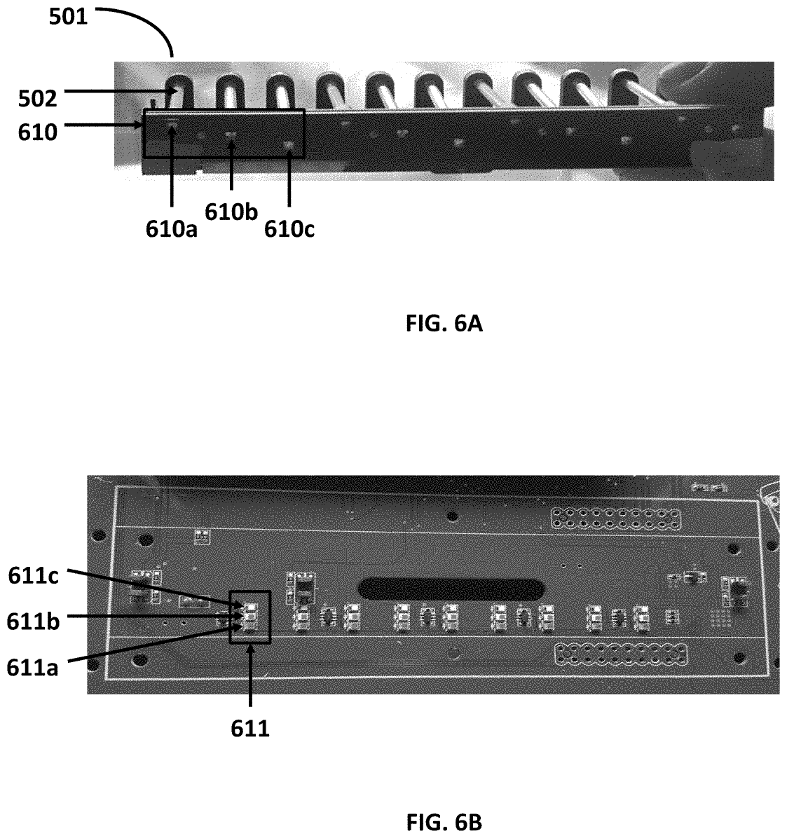

A carriage may comprise one or more optical filters (e.g., excitation or emission filters) and one or more light pipes. FIG. 6A shows a carriage comprising one or more excitation filters 610a (red), 610b (yellow), and 610c (blue). A carriage may also comprise one or more emission filters. A light pipe may extend from an optical filter (e.g., an excitation filter) to an assay tube containing a sample.

An analytic device may comprise any useful optical filters (e.g., excitation and/or emission filters). Filters may be optical bandpass filters (e.g., optical interference films) having a bandpass at a frequency that may be optimal for one or more of (i) the excitation wavelength of a fluorophore or dye, and (ii) the emission wavelength of a fluorophore or dye. A filter may substantially attenuate non-bandpass frequencies to prevent transmission of undesirable light. For example, when using SYBR Green dye, an excitation filter bandpass may center around a wavelength of 485 nm, and an emission filter bandpass may center around a wavelength of 555 nm. An optical filter (e.g., an excitation filter and/or an emission filter) may be tilted (e.g., a plane containing the filter may be disposed at an angle) relative to a light path.

### Excitation Source

An analytic device may comprise one or more excitation sources. An excitation source may be disposed on a carriage (e.g., a moving carriage, as described herein) and may be configured to deliver excitation energy to a sample (e.g., a sample in an assay tube) through an excitation filter and a light path. For an analytic device comprising a moving carriage, a single excitation source disposed on the carriage may be configured to deliver excitation energy to two or more samples (e.g., two or more samples in two or more assay tubes) through the same excitation filter and light path (e.g., as the moving carriage aligns the excitation source and light path with different assay tubes containing different samples). As shown in FIG. 6B, an analytic device may have a dedicated set 611 of excitation sources 611a (blue), 611b (yellow), and 611c (red) for each assay tube.

An excitation source may comprise a Light Emitting Diode (LED) or an array of LEDs (e.g., a set of single-color LEDs). An LED may have any useful size, shape, wavelength, or other characteristic. An LED may be a high power LED that may emit greater than or equal to about 1 mW of excitation energy. A high power LED may emit at least about 5 mW of excitation energy. An LED or an array of LEDs may emit, for example, about 50 mW of excitation energy. An array of high-powered LEDs may be used that draws, for example, about 10 watts of energy or less, or about 10 watts of energy or more. The total power draw may depend on the power of each LED and the number of LEDs in the array. The use of LEDs in an analytic device as an excitation source may be beneficial, for example, because an LED array may result in a significant reduction in power requirement over other light sources such as halogen light sources. An excitation source may use a power of about 1 microwatt (μW) or less. Alternatively, an excitation source may use a power of about 1 microwatt (μW), about 5 μW, about 25 μW, about 50 μW, about 100 μW, about 1 milliwatt (mW), about 5 mW, about 25 mW, about 50 mW, about 100 mW, about 1 W, about 5 W, about 50 W, or about 100 W or more, individually or when in used in an array. In some cases, a cooling device such as, but not limited to, a heat sink or fan may be used to cool the excitation source or a component thereof.

An excitation source may comprise an organic LED (OLED) or an array of OLEDs. An OLED may have any useful size, shape, wavelength, or other characteristic. An OLED may provide luminescence over a large area, for example, to provide excitation energy to multiple assay tubes simultaneously. Scatter or cross-talk light between multiple sample wells (e.g., seats or housings for assay tubes) for such an OLED may be reduced by overlaying a mask on the OLED or by patterning the luminescence of the OLED to operatively align with the multiple sample wells. An OLED may be a low power consumption device. An OLED may include a small-molecule OLED and/or a polymer-based OLED also known as a Light-Emitting Polymer (LEP). A small-molecule OLED that is deposited on a substrate may be used. An OLED that is deposited on a surface by vapor-deposition technique may be used. An OLED may also be deposited on a surface by, for example, silk-screening. An LEP may be used that is deposited by, for example, via solvent coating.

An excitation source may comprise an array of LEDs or OLEDs 611a-611c (e.g., multiple single-color LEDs). The array may be constructed and arranged in any configuration. For example, the excitation sources in an array may be arranged linearly along the axis of movement of a moving carriage. Alternatively, as shown in FIG. 6B, the excitation sources in an array may be arranged linearly perpendicular to the axis of movement of a moving carriage. In such a configuration, the light paths 502 may be disposed at an angle relative to the base of the moving carriage. A light path extending from the base of the moving carriage (e.g., from an excitation filter disposed in the base of the moving carriage) may be perpendicular to the base of the carriage, or not perpendicular to the base of the carriage (e.g., at an angle other than 90 degrees to the base of the carriage).

One or more lenses may be used to direct, re-direct, focus, disperse, or collimate excitation or emission energy. For example, a lens may be used to focus excitation energy onto a sample (e.g., a sample in an assay tube). In another example, a lens may be used to collimate excitation energy from an excitation source. Non-limiting examples of lenses that may be used include a biconvex lens, a plano-convex lens, a positive meniscus lens, a negative meniscus lens, a plano-concave lens, a biconcave lens, a Fresnel lens, a cylindrical lens, a lenticular lens, and a gradient index lens. For example, a Fresnel lens may be used to collimate excitation energy from an excitation source and direct the excitation energy into a light path. A Fresnel lens may be made much thinner than a comparable plano-convex lens, in some cases taking the form of a flat sheet, which may be advantageous for producing a portable analytic device.

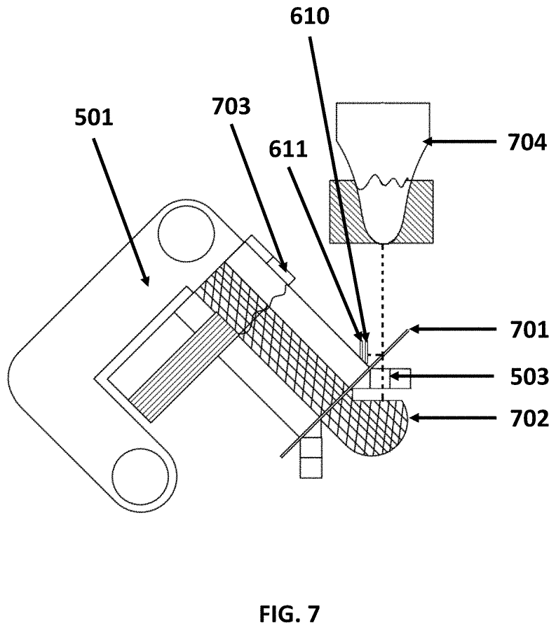

FIG. 7 shows an additional configuration for moving carriage 501 in which excitation source 611, excitation filter 610, dichroic beam splitter 701, emission filter 503, and detector 702 are disposed on moving carriage 501. Excitation source 611, excitation filter 610, dichroic beam splitter 701, and emission filter 503 may be disposed on a rotating pinion mechanism 703 such that as moving carriage 501 aligns with each sample, the pinion mechanism may be used to rotate the optical components 611, 610, 701, and 503 to provide to a desired excitation energy to a sample (e.g., a sample in an assay tube), and detect an emission energy from the sample 704.

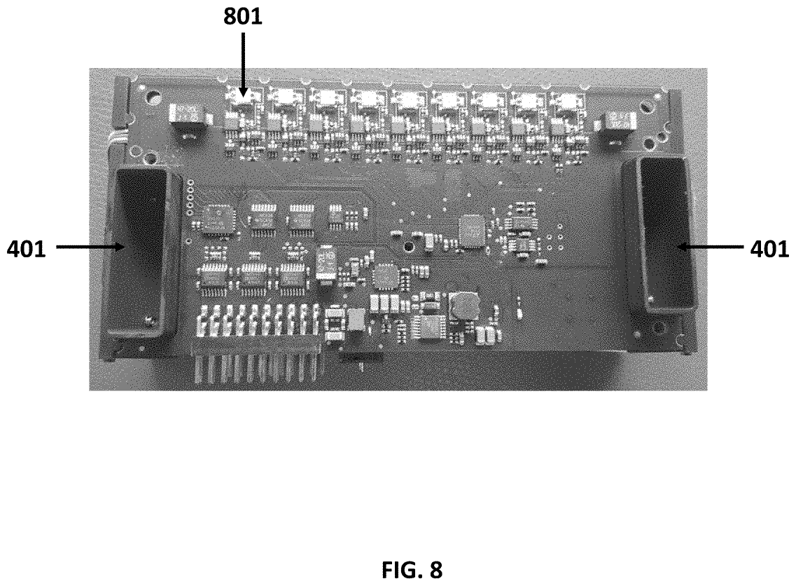

The analytic device may also comprise a detector such as detector 801, as shown in FIG. 8. The detector may be configured to receive emission energy from a sample (e.g., a sample in an assay tube), and possibly through an emission filter. Accordingly, the detector may comprise any suitable photodetector, such as, for example, an optical detector, a photoresistor, a photovoltaic cell, a photo diode, a phototube, a photomultiplier tube, a charge coupled device (CCD) camera, a complementary metal oxide semiconductor (CMOS), or any combination thereof. Emission energy may be produced by any suitable source, such as, for example, by the excitation of a component of a sample in an assay tube (e.g., an excitable fluorophore). A detector may be configured to selectively receive emission energy from a sample (e.g., energy of a particular wavelength or intensity). A detector may comprise a plurality of detectors (e.g., a series of photodetectors, each configured to receive a light beam having a different wavelength than the light beams received by the other photodetectors).

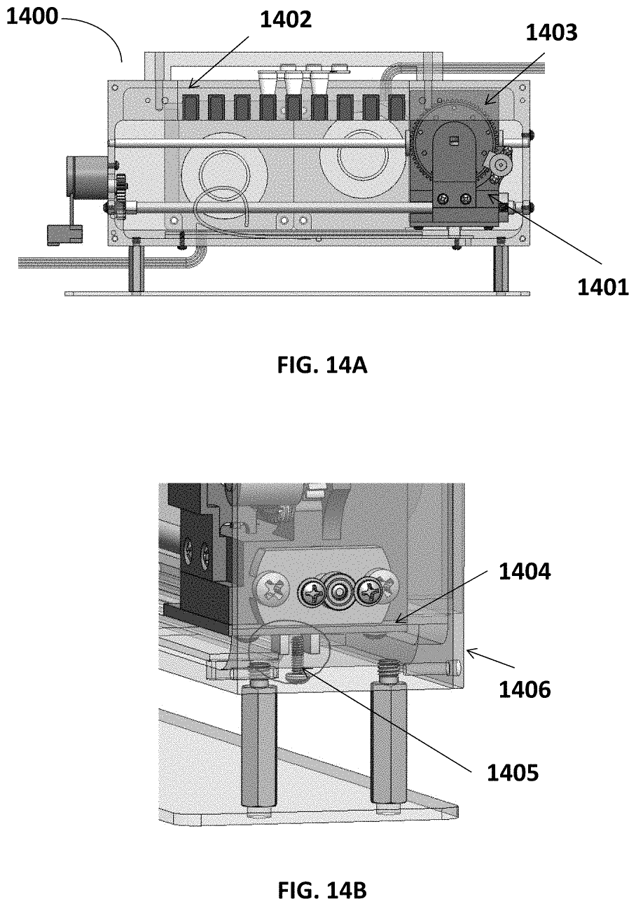

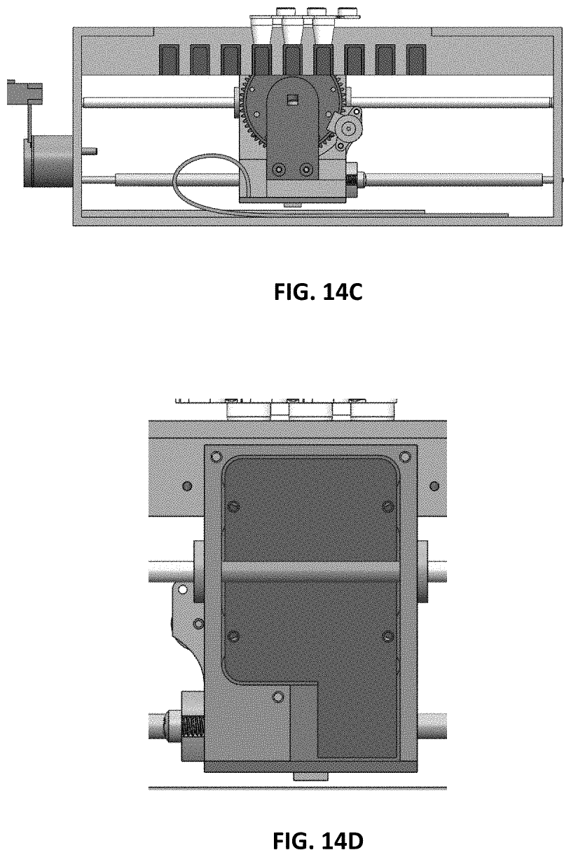

A movable carriage may comprise a wheel-shaped (or circular) component to carry one or more optical elements, such as filters. As an alternative or in addition to, the wheel-shaped component can include a mirror, light source (e.g., an LED, a single pixel LED, or a multi-pixel LED), prism, lens, or any combination thereof. The movable carriage can be configured to move in a linear path and stopped at a specific position. For example, the movable carriage can be configured to move along the axis of heating blocks and stopped at each heating block for data acquisition from a sample tube inserted into each heating block. The wheel-shaped component inside the movable carriage may be movable along the wheel axle to switch between different filters. For example, FIG. 14A shows a front view of a movable carriage 1401 inside a portable device 1400. In this example device, the wheel-shaped component 1403 of the movable carriage 1401 carries 9 pairs of filters (a pair of filter comprises an excitation filter and an emission filter). The movable carriage can move along the different heating blocks 1402. FIG. 14B shows a zoom-in view of a portion of the movable carriage. The bottom PCB 1404 may comprise a break beam switch. The chassis 1406 can comprise two screws to trigger beam switch to stop carriage from hitting chassis walls. One screw 1405 is shown in FIG. 14B. FIG. 14C shows an additional front view of the example movable carriage stopped at a different position inside a portable device. FIG. 14D shows a back view of the example movable carriage.

The wheel-shaped component can have other shapes. For example, the elements of such wheel-shaped component may be included in a component that is triangular, square, rectangular, pentagonal, hexagonal, or any other shape or combination of shapes thereof.

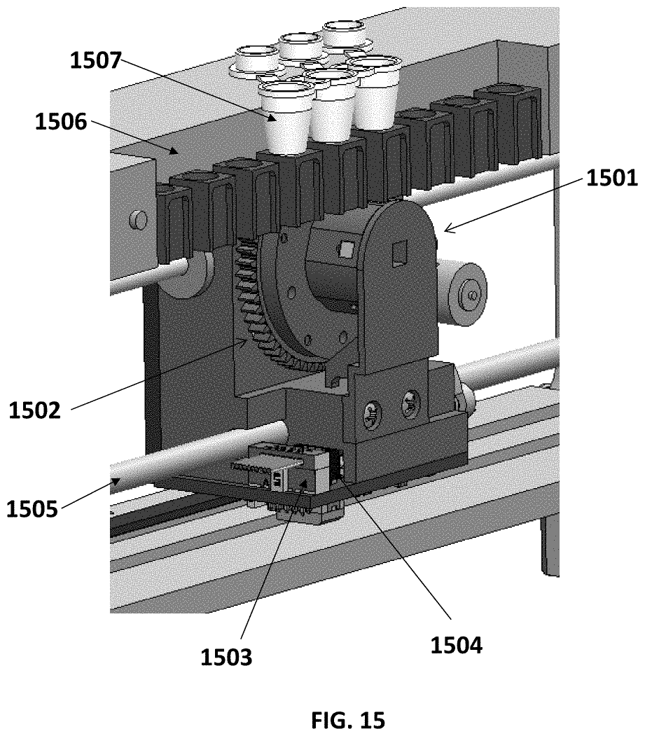

FIG. 15 shows a zoom-in view of an example movable carriage 1501 having a wheel-shaped component 1502. The bottom portion of the movable carriage can comprise a ribbon wire 1503 and an actuator (e.g., stepper motor) 1504. The stepper motor 1504 may be used to move the movable carriage along a guide 1505 among the sample stations 1506. A given one of the sample stations 1506 may include a vial 1507 having a solution containing a biological sample and reagents necessary for sample processing (e.g., polymerase chain reaction (PCR)). The movable carriage 1501 may include another actuator (e.g., stepper motor) for rotating the movable carriage 1501 along an axis orthogonal to the guide 1505.

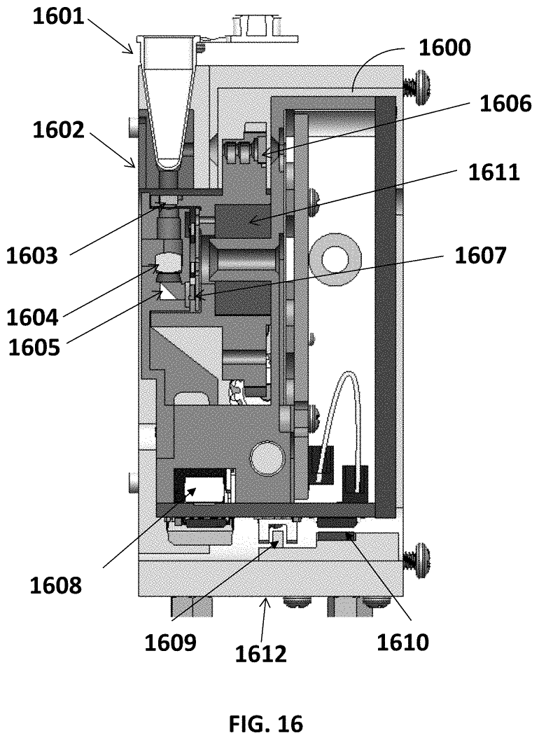

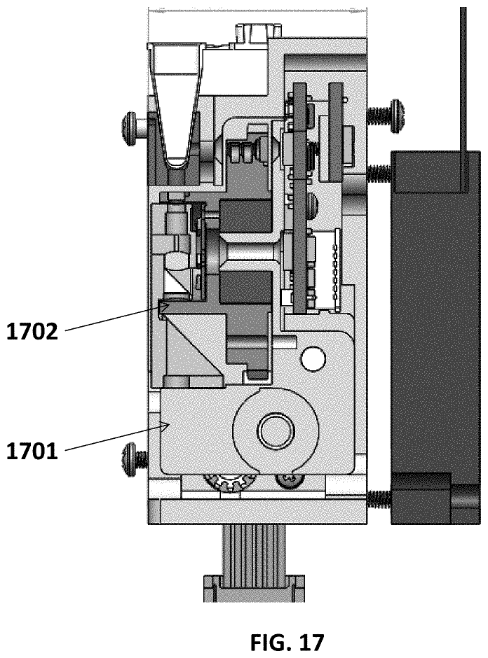

FIG. 16 shows a side view of the internal mechanism of the example movable carriage 1600. The movable carriage can comprise an optical system having an excitation filter 1603, a lens 1604, a mirror 1605, an emission filter 1606, and a light source 1607 (e.g., LED). The movable carriage can comprise one or more magnetic pieces 1611. The movable carriage may comprise multiple excitation filters, emission filters, and light sources. Each light source may be configured to be used with a given pair of excitation filter and emission filter for data acquisition from a sample tube 1601 inserted in a heating block 1602. Shown in FIG. 16 is an example of one optical system having one pair of excitation and emission filters. When the wheel-shaped component moves around the wheel axle, another optical system having another pair of excitation and emission filters and another light source can be lined up with the sample tube for data acquisition. The movable carriage can comprise at least 1, 2, 3, 4, 5, 6, 7, 8, 9, 10, 11, 12, 13, 14, 15, 16, 17, 18, 19, 20 or more filters. The movable carriage can comprise at least one pair, two pairs, three pairs, four pairs, five pairs, six pairs, seven pairs, eight pairs, nine pairs, ten pairs, eleven pairs, twelve pairs, thirteen pairs, fourteen pairs, fifteen pairs, or more pairs of filters. The movable carriage can further comprise a big capacitor 1608. The chassis 1612 of the device can comprise a flag to trigger photo interrupter. The chassis 1612 can comprise a magnetic strip and linear encoder (e.g., a liner encoder having a 0.4 mm gap). The movable carriage can be built with various materials or combinations of materials. For example, shown in FIG. 17, the part 1701 of the movable carriage can be built with metal. The part carrying the optical system 1702 may be built with black dyed micro fine 3D print. The detector board may be fully enclosed for EMI shielding.

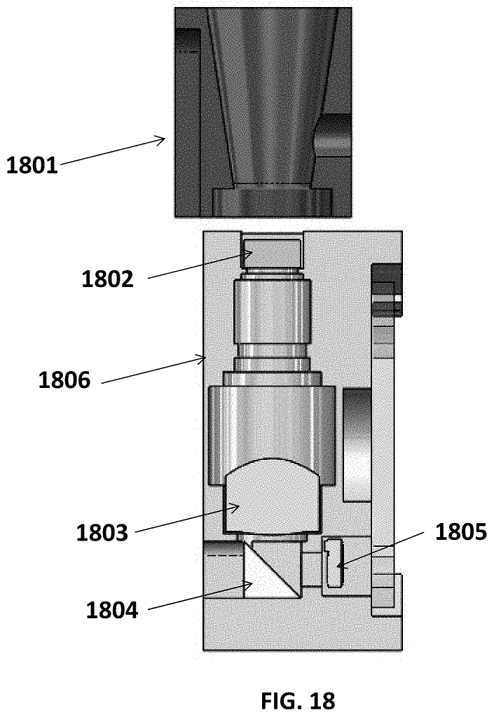

FIG. 18 shows a zoom-in view of an example mechanism of an optical system of the movable carriage. The lens 1803 can be made of various materials, for example, glass or polycarbonate. The lens 1803 may be mounted in a non-rotating part of the hub 1806 of the wheel-shaped component. The light source (or excitation source) 1805 can be a LED light. The filter 1802 can be an excitation filter. The filter 1802 may provide transmission of a desired excitation wavelength. For example, the light transmitted from the excitation filter may have a center wavelength of at least about 390 nanometers (nm), 434 nm, 445 nm, 469 nm, 475 nm, 497 nm, 542 nm, 559 nm, or 565 nm. The optical system can further comprise a fold mirror 1804. The distance between the light source 1805 and the fold mirror 1804 can vary. Shown in FIG. 18, the part 1801 is a heating block. In addition, the optical system can comprise an emission filter. The emission filter can provide transmission of a desired emission wavelength. For example, the light transmitted from the emission filter may have a center wavelength of at least about 460 nm, 479 nm, 510 nm, 525 nm, 530 nm, 535 nm, 620 nm, or 630 nm. In some cases, the optical system inside a movable carriage may comprise one or more dichroic filters.

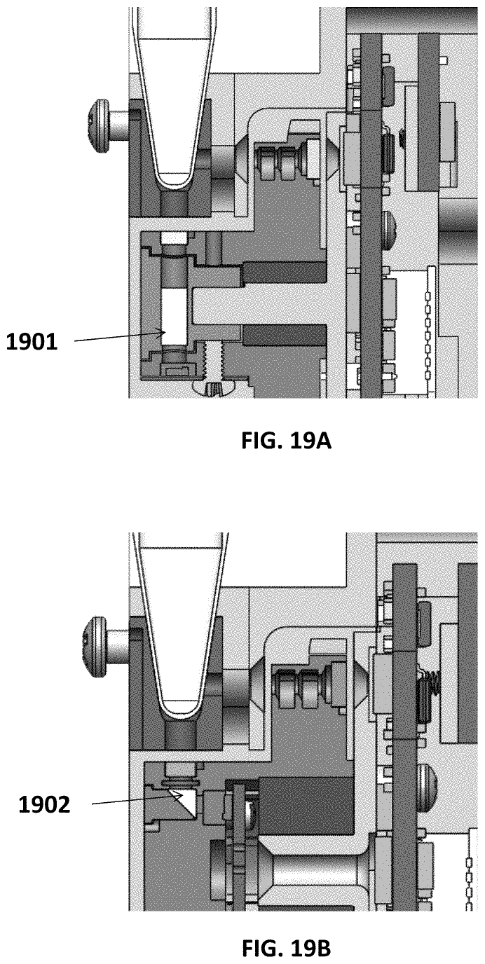

The optical system may comprise different components and can be assembled in different configurations. FIGS. 19A and 19B show two additional examples of the optical systems inside of a movable carriage. For example, an optical system of the movable carriage may not comprise a mirror and lens. An optical system may comprise a light path 1901 that allow the light from a light source to reach an excitation filter. For another example, an optical system may comprise a prism 1902 to allow the light from a light source to reach the excitation filter.

Different configurations of the optical systems may result in different properties of the system as demonstrated by parameters such as power to vial, moving carriage baseline, signal to noise ratio (SNR), etc. As used herein, the SNR can be defined using the following equation:

$$SNR = \frac{Total power on detector}{Power on filter outside \times degrees that reaches the detector}$$

where, x is the incidence angle of a light.

Typically, x may be 25 degrees on excitation and 15 degrees on emission. “Power to vial” refers to the total optical power making it into the vial that is available for excitation of fluorescent probes. “Moving carriage baseline,” as used herein, refers to a baseline used for comparing different configurations of the optical system. Example data shown in the present disclosure are baselined against the configuration without a wheel-shaped component, for example, as shown in FIG. 7 and FIG. 8. Using the parameters described herein, the properties of different configurations can be tested by excitation simulation. For example, an optical system can have a power to vial value of about 2%, 3%, 4%, 5%, 6%, 7%, 8%, 9%, 10% or more. The optical system can be 1 fold, 1.5 fold, 2 fold, 2.5 fold, 3 fold, 3.5 fold, 4 fold, 5 fold, 6 fold, 7 fold, 8 fold, 9 fold, 10 fold, 15 fold, 20 fold, or more efficient than the moving carriage baseline. The SNR of the optical system can be at least 1,000, 1,500, 2,000, 2,500, 3,000, 3,500, 4,000, 5,000 or more. In some case, the SNR of the optical system can be at least 100, 150, 200, 250, 300, 350, 400, 450, 500 or more. For example, a configuration shown in FIG. 16 have a power to vial value of 5.8%, 2 to 20 fold more efficient than the moving carriage baseline, and have a SNR value of about 2,000. FIGS. 20A-C and 21A-C show example simulation results of the optical system.

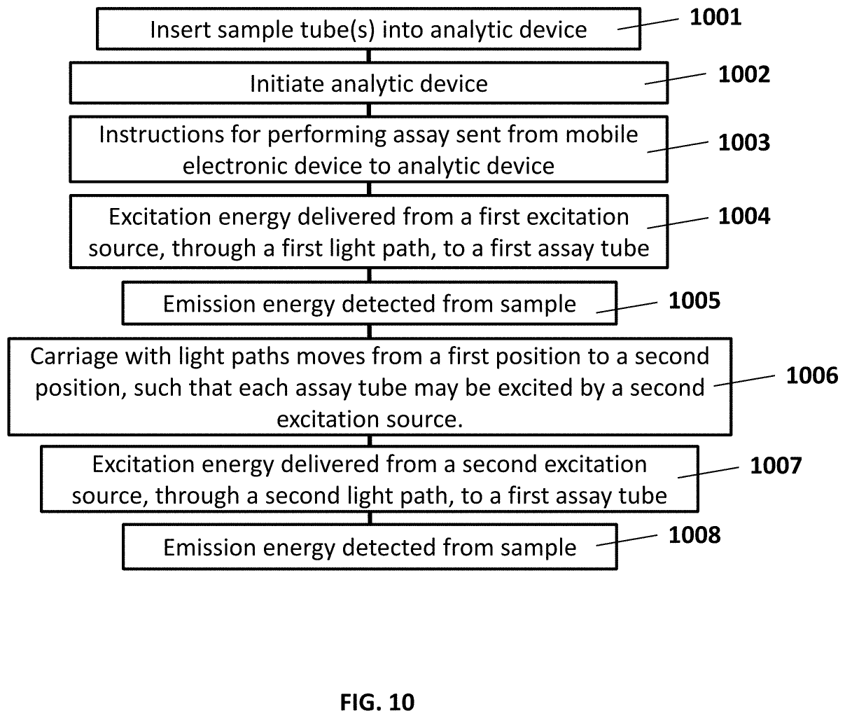

FIG. 10 shows an example process flow for the analytic device of FIGS. 1A-1B. In a first operation 1001, lid 101 of housing 100 is opened, and a user inserts one or more assay tubes each containing a sample into the analytic device. In a second operation 1002, the user initiates the analytic device by pressing power button 103 located on housing 100. In a third operation 1003, the user provides instructions for performing an amplification reaction (e.g., a thermal cycling assay). The instructions may be provided using an application on a mobile electronic device (e.g., which may be physically detached from the analytic device, integrated into the analytic device, or removably disposed in or on the analytic device, for example in a housing or groove of the analytic device). Instructions provided to the application may then be communicated to the analytic device (e.g., via a wireless connection, as described herein). In a fourth operation 1004, the analytic device is initiated, and an excitation energy is delivered from excitation source 611, through excitation filter 610, through light path 502, to a first assay tube. In a fifth operation 1005, emission energy from the sample in the first assay tube is delivered from the sample through emission filter 503 to detector 801. In a sixth operation 1006, a moving carriage comprising excitation source 611, excitation filter 610, and emission filter 503 may move to a second position (e.g., aligning light path 502 with a second assay tube). In a seventh operation 1007, excitation energy is delivered from a second excitation source, through a second excitation filter, through a second light path, to the first assay tube. In an eighth operation 1008, emission energy from the sample in the first assay tube is delivered from the sample through a second emission filter and to detector 801.

### Samples

A variety of samples (e.g., biological samples) may be analyzed. A sample may be obtained invasively (e.g., tissue biopsy) or non-invasively (e.g., venipuncture). The sample may be an environmental sample. The sample may be a water sample (e.g., a water sample obtained from a lake, stream, river, estuary, bay, or ocean). The sample may be a soil sample. The sample may be a tissue or fluid sample from a subject, such as saliva, semen, blood (e.g., whole blood), serum, synovial fluid, tear, urine, or plasma. The sample may be a tissue sample, such as a skin sample or tumor sample. The sample may be obtained from a portion of an organ of a subject. The sample may be a cellular sample. The sample may be a cell-free sample (e.g., a plasma sample comprising cell-free analytes or nucleic acids). A sample may be a solid sample or a liquid sample. A sample may be a biological sample or a non-biological sample. A sample may comprise an in-vitro sample or an ex-vivo sample. Non-limiting examples of a sample include an amniotic fluid, bile, bacterial sample, breast milk, buffy coat, cells, cerebrospinal fluid, chromatin DNA, ejaculate, nucleic acids, plant-derived materials, RNA, saliva, semen, blood, serum, soil, synovial fluid, tears, tissue, urine, water, whole blood or plasma, and/or any combination and/or any fraction thereof. In one example, the sample may be a plasma sample that may comprise DNA. In another example, the sample may comprise a cell sample that may comprise cell-free DNA.

A sample may be a mammalian sample. For example, a sample may be a human sample. Alternatively, a sample may be a non-human animal sample. Non-limiting examples of a non-human sample include a cat sample, a dog sample, a goat sample, a guinea pig sample, a hamster sample, a mouse sample, a pig sample, a non-human primate sample (e.g., a gorilla sample, an ape sample, an orangutan sample, a lemur sample, or a baboon sample), a rat sample, a sheep sample, a cow sample, and a zebrafish sample.

The devices and methods disclosed herein may be useful for analyzing nucleic acids (e.g., circulating and/or cell-free DNA fragments). Nucleic acids may be derived from eukaryotic cells, prokaryotic cells, or non-cellular sources (e.g., viral particles). A nucleic acid may refer to a substance whose molecules consist of many nucleotides linked in a long chain. Non-limiting examples of the nucleic acid include an artificial nucleic acid analog (e.g., a peptide nucleic acid, a morpholino oligomer, a locked nucleic acid, a glycol nucleic acid, or a threose nucleic acid), chromatin, niRNA, cDNA, DNA, single stranded DNA, double stranded DNA, genomic DNA, plasmid DNA, or RNA. A nucleic acid may be double stranded or single stranded. A sample may comprise a nucleic acid that may be intracellular. Alternatively, a sample may comprise a nucleic acid that may be extracellular (e.g., cell-free). A sample may comprise a nucleic acid (e.g., chromatin) that may be fragmented.

### Sample Processing

The present disclosure provides methods and systems for processing samples in assay tubes. Samples, such as nucleic acid samples, may be disposed in assay tubes and processed simultaneously or separately. The sample may be processed simultaneously but independent from one another. For example, a first sample in a first assay tube is subjected to different processing conditions then a second sample in a second assay tube. Alternatively, the first sample and the second sample may be subjected to the same or substantially the same processing conditions.

### Sample Preparation Systems

Analysis of biological sample-derived materials may not occur until the sample is processed through numerous pre-analysis steps. Often, the preparation process can be time consuming, laborious, and can be subject to human error. For example, immuno- and molecular-biological diagnostic assays on clinical samples, such as blood or tissue cells, may need separation of the molecules of interest from the crude sample by disrupting or lysing the cells to release such molecules including proteins and nucleic acids (i.e., DNA and RNA) of interest, followed by purification of such proteins and/or nucleic acids. Only after performing processing steps can analysis of the molecules of interest begin. Additionally, protocols used for the actual analysis of the samples may use numerous more steps before useful data is obtained. The present disclosure provides devices, systems, methods for the automated or substantially automated processing of biological samples.

The present disclosure also provides devices, systems and methods for sample preparation and processing. Such devices, systems and methods may permit the automated processing of biological samples in a lab-free environment. Devices and systems of the present disclosure may be portable, allowing users to employ such devices in remote locations, for example.

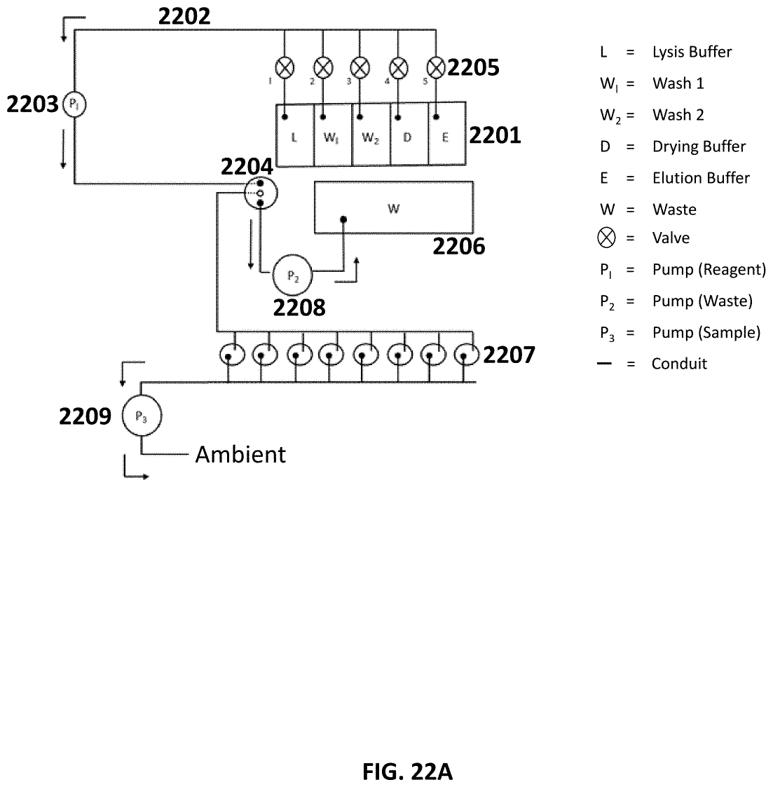

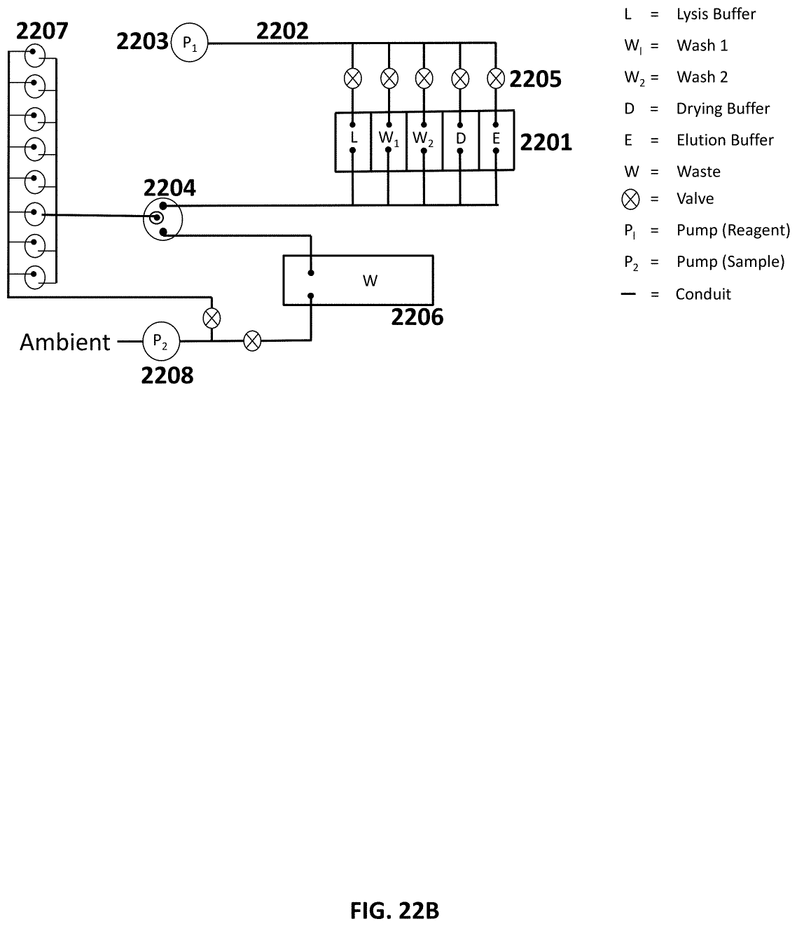

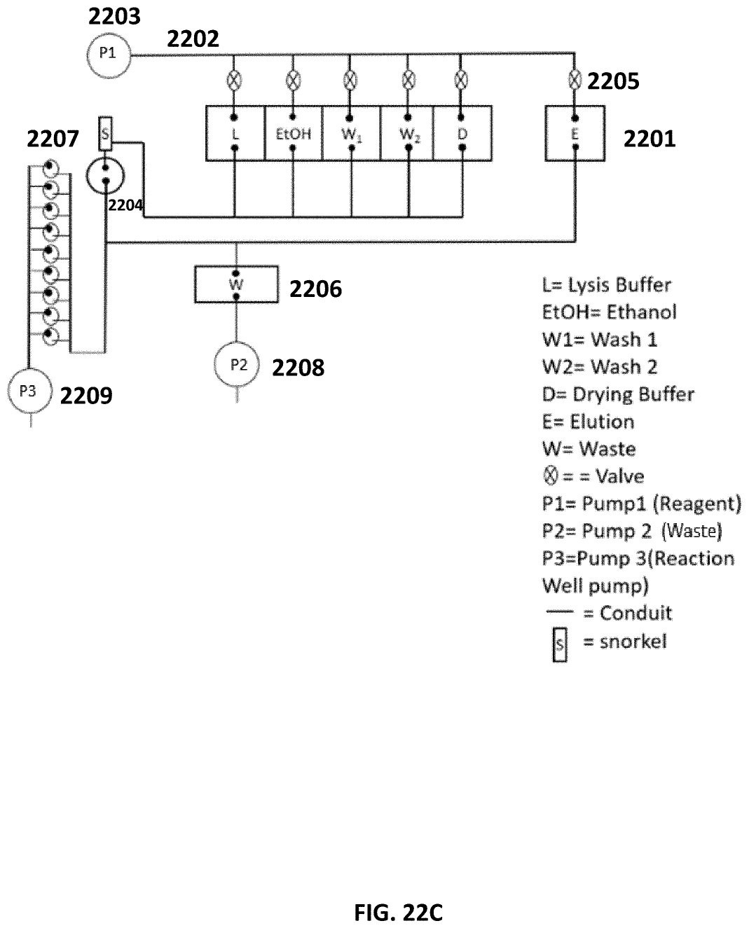

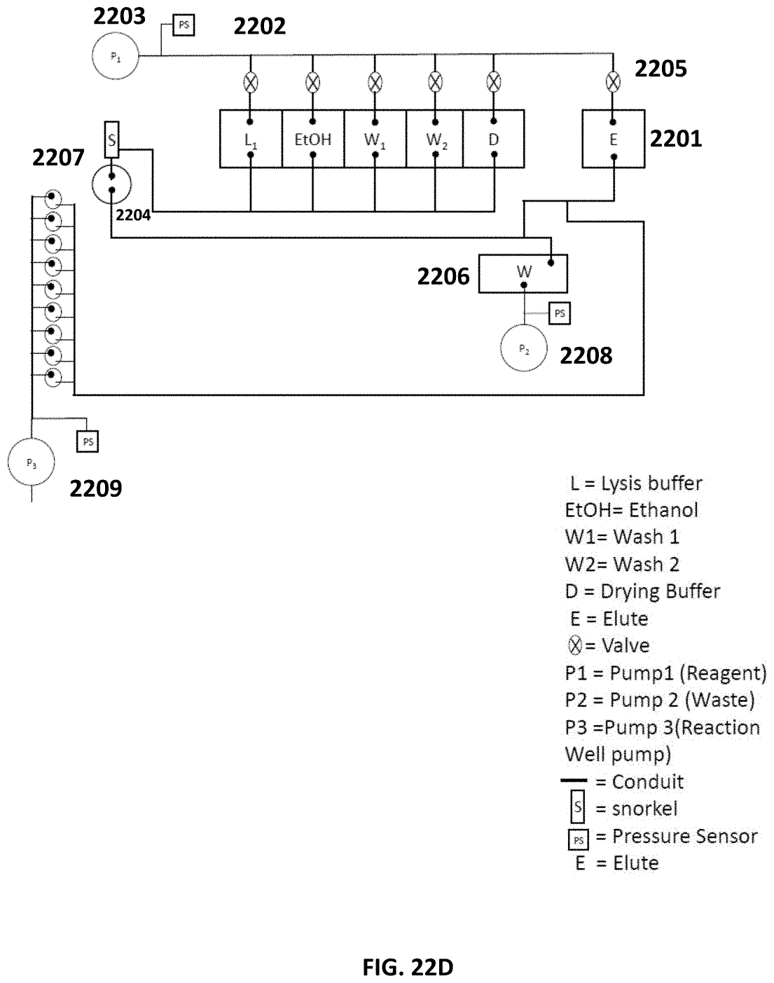

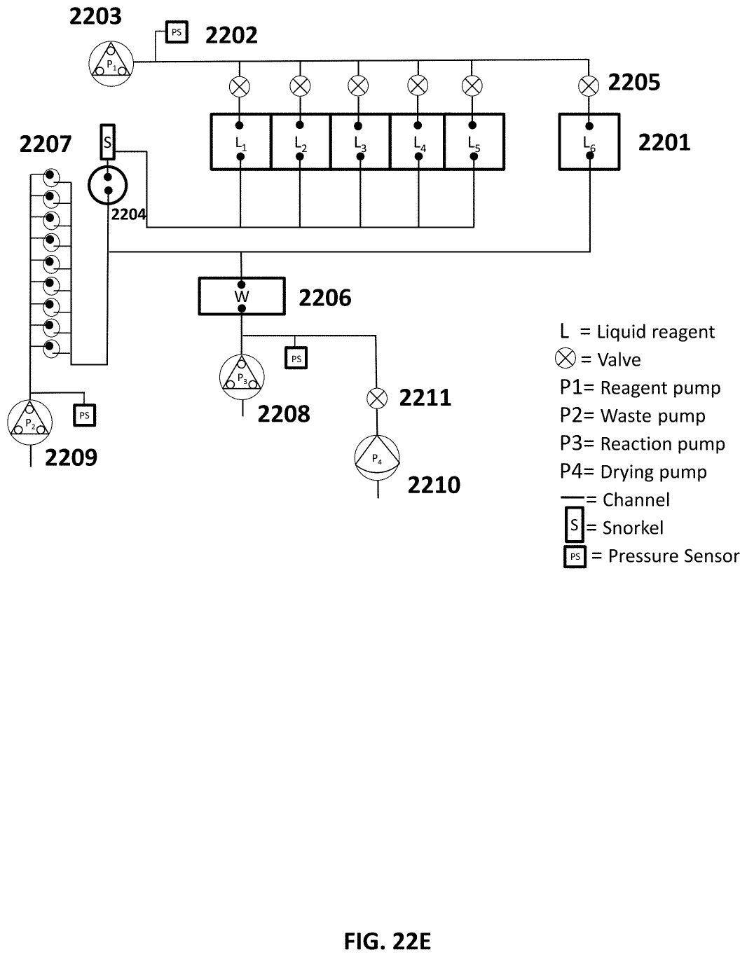

FIGS. 22A-22E schematically illustrate examples of systems for sample preparation and/or analysis.

FIG. 22A schematically illustrates a system for sample preparation. The system includes reagent chambers 2201 that are fluidly connected by conduits 2202 to a first pump 2203 capable of applying a draw pressure (or pressure drop) to transfer fluid from the reagent chambers to a sample chamber 2204. The draw pressure may be selectively applied to one or more chambers by opening valves 2205 disposed along the conduit between the reagent chamber and the pump. Fluid from the sample chamber may be transferred to the waste chamber 2206, or to one or more assay tubes 2207 for further analysis, using a second pump 2208 or third pump 2209.

FIG. 22B schematically illustrates another system for sample preparation. The system includes reagent chambers 2201 that are fluidly connected by conduits 2202 to a first pump 2203 capable of applying a positive pressure (e.g., pressure that is greater than a reference pressure, such as ambient pressure) to push fluid from the reagent chambers to a sample chamber 2204. In this arrangement, as compared with the system of FIG. 22A, the first pump does not contact the fluid in the reagent chambers. The positive pressure may be selectively applied to one or more chambers by opening valves 2205 disposed along the conduit between the reagent chamber and the pump. Fluid from the sample chamber may be transferred to the waste chamber 2206, or to one or more assay tubes 2207 for further analysis, using a second pump 2208. Yet another system comprising a first pump as shown in FIG. 22A (e.g., configured to draw fluid from the reagent chambers) and a second pump as shown in FIG. 22B (e.g., configured to draw fluid from the sample chamber) is also contemplated.

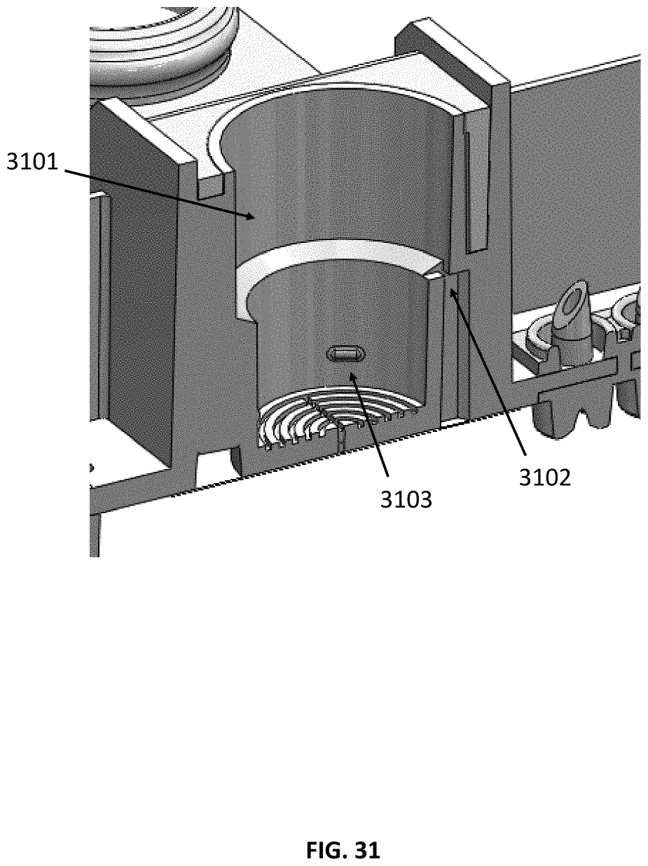

FIG. 22C schematically illustrates another system for sample preparation. The system includes reagent chambers 2201 that are fluidly connected by conduits 2202 to a first pump 2203 capable of applying a positive pressure to push fluid from the reagent chambers to a sample chamber 2204. In this arrangement, the cartridge comprises six reagent chambers. Besides the five reagent chambers similar to the systems shown in FIGS. 22A and 22B, an additional reagent chamber is included for an additional reagent, for example, ethanol. This additional reagent chamber may include an elution buffer, for example. Also in this arrangement, the first pump 2203 does not contact the fluid in the reagent chambers. The positive pressure may be selectively applied to one or more chambers by opening valves 2205 disposed along the conduit between the reagent chamber and the pump. A sample chamber 2204 can be connected to reagent chambers through one or more conduits. As shown here in FIG. 22C, a main conduit connecting the sample chamber and the reagent chambers can further comprise a snorkel. Fluid from the sample chamber 2204 may be transferred to the waste chamber 2206 using a second pump 2208, or to one or more assay tubes 2207 using a third pump 2209 for analysis. As an alternative, a single pump and one or more valves may be used to draw fluid from the sample chamber 2204 into the waste chamber 2206 or the one or more assay tubes 2207 (see, e.g., FIG. 22B). An example of a sample chamber 3201 connected to a snorkel 3102 is shown in FIG. 31. The snorkel 3102 can have a ventilating function and it can connect the sample chamber 3101 to the ambient air. The part 3103 shown in this figure is a pump to capture a filter stack. Examples of the filter stack include, but are not limited to, hydrophilic porous support, porous Glass filter, or hydrophobic porous support.

FIG. 22D schematically illustrates another system for sample preparation. The system includes reagent chambers 2201 that are fluidly connected by conduits 2202 to a first pump 2203 capable of applying a positive pressure to push fluid from the reagent chambers to a sample chamber 2204. Similar to the system shown in FIG. 22C, in this arrangement, the cartridge comprises six reagent chambers containing five reagent chambers similar to the systems shown in FIGS. 22A and 22B and an additional reagent chamber. In this arrangement, the first pump 2203 does not contact the fluid in the reagent chambers. The positive pressure may be selectively applied to one or more chambers by opening valves 2205 disposed along the conduit between the reagent chamber and the pump. A sample chamber 2204 can be connected to reagent chambers through one or more conduits. A main conduit connecting the sample chamber and the reagent chambers can further comprise a snorkel. Fluid from the sample chamber 2204 may be transferred to the waste chamber 2206 using a second pump 2208, or to one or more assay tubes 2207 using a third pump 2209 for analysis. The second pump 2208 may be used for drying the waste chamber 2206 as well. As an alternative, a single pump and one or more valves may be used to draw fluid from the sample chamber 2204 into the waste chamber 2206 or the one or more assay tubes 2207.

FIG. 22E schematically illustrates another system for sample preparation. The system includes reagent chambers 2201 that are fluidly connected by conduits 2202 to a first pump 2203 capable of applying a positive pressure to push fluid from the reagent chambers to a sample chamber 2204. Similar to the system shown in FIG. 22D, in this arrangement, the cartridge comprises six reagent chambers containing five reagent chambers similar to the systems shown in FIGS. 22A and 22B and an additional reagent chamber. In this arrangement, the first pump 2203 does not contact the fluid in the reagent chambers. The positive pressure may be selectively applied to one or more chambers by opening valves 2205 disposed along the conduit between the reagent chamber and the pump. A sample chamber 2204 can be connected to reagent chambers through one or more conduits. A main conduit connecting the sample chamber and the reagent chambers can further comprise a snorkel. Fluid from the sample chamber 2204 may be transferred to the waste chamber 2206 using a second pump 2208, or to one or more assay tubes 2207 using a third pump 2209 for analysis. A fourth pump 2210 may be connected to the waste chamber 2206 for drying. A conduit connecting the fourth pump 2210 to the waste chamber 2206 may comprise a valve 2211 and/or a pressure sensor. As an alternative, a single pump and one or more valves may be used to draw fluid from the sample chamber 2204 into the waste chamber 2206 or the one or more assay tubes 2207.

Although FIGS. 22A-22E illustrate examples of pump and valve configurations, Various pump and/or valve configurations may be used, such as, for example, “wet pumps” (e.g., pumps configured to contact a fluid) and/or “dry pumps” (e.g., pumps configured to not contact a fluid) may be used in systems of the present disclosure. In addition, other units for effecting fluid flow may be used, such as one or more compressors and/or one or more compressors together with one or more pumps.

The pumps 2203, 2208, 2209 and 2210 may be configured to supply a negative pressure (e.g., vacuum). As an alternative, the pumps 2203, 2208, 2209 and 2210 may be configured to supply positive pressure. As another alternative, the pumps 2203, 2208, 2209 and 2210 may be configured to supply both negative pressure and positive pressure in alternative modes of operations, which may be used to subject a fluid along a first direction and subsequently along a second direction different from (e.g., opposite of) the first direction. The pumps 2203, 2208, 2209 and 2210 may be multi-directional (e.g., bi-directional) pumps, each configured to operate in a first mode in which negative pressure is applied to a fluid flow path and a second mode in which positive pressure is applied to the fluid flow path. Such pumps may have other modes in which a range of pressures (or pressure drops) are applied.

The systems described herein may comprise various numbers of pumps. In some cases, the systems comprise 2 or 3 pumps as illustrated in FIGS. 22A-22D. In some cases, the systems comprise 4 pumps, as illustrated in FIG. 22E. In some other cases, the systems comprise one pump. In some other cases, the systems comprise 5, 6, 7, 8, 9, 10, or more pumps. In some cases, the systems comprise at least 1, at least 2, at least 3, at least 4, at least 5, at least 6, at least 7, at least 8, at least 9, at least 10, at least 20, at least 30, at least 40, or more pumps.

The valves 2205 or 2211 may be actuated by various approaches. Such approaches include pneumatic actuation, such as with the aid of positive pressure or negative pressure from a source of positive pressure or negative pressure, respectively. Positive pressure may be provided using one or more compressors. Negative pressure may be provided using one or more pumps. In another approach, valves may be actuated using electrothermal heating. For example, a valve can be a shape memory valve. A shape memory valve may refer to any type of valve that comprises a material that “remembers” its original shape and is capable of returning to its pre-deformed shape when heated. In some cases, the shape memory valve can comprise a nitinol or Nickel Titanium wire that actuates a seal during contraction upon electrothermal heating. In some cases, the shape memory valve can comprise a copper-aluminum-nickel wire that actuates a seal during contraction upon electrothermal heating. In yet another approach, valves may be actuated using electromechanical units. For example, the valve can be a solenoid valve. An electromechanical valve can refer to any type of valve that is controlled by an electric current (e.g., through a solenoid). In some cases, the solenoid valve may be a latching solenoid valve. In the case of a two-port valve, flow may be switched on or off. In the case of a three-port valve, outflow may be switched between any or both of the one or more outlet ports. The numbers of valves shown in FIGS. 22A-22E are non-limiting examples. The systems may comprise various numbers of valves. In some cases, the systems do not comprise any valve. In some cases, the systems comprise more valves than the systems shown in FIGS. 22A-22E. For example, the systems may comprise at least 1, at least 2, at least 3, at least 4, at least 5, at least 6, at least 7, at least 8, at least 9, at least 10, at least 20, at least 30, at least 40, or more valves.

The conduits may have various dimensions. In some examples, the conduits 102 have dimensions on the order of micrometers. In such cases, the conduits 102 may be part of a microfluidic device.

Although the systems of FIGS. 22A-22E include a certain number of reagent chambers, systems of the present disclosure may include at least 1, 2, 3, 4, 5, 6, 7, 8, 9, 10, 15, 20, or more chambers, which may be reagents chambers. A given chamber may house or contain a reagent. As an alternative or in addition to, a given chamber may be used for conducting a reaction or mixing.

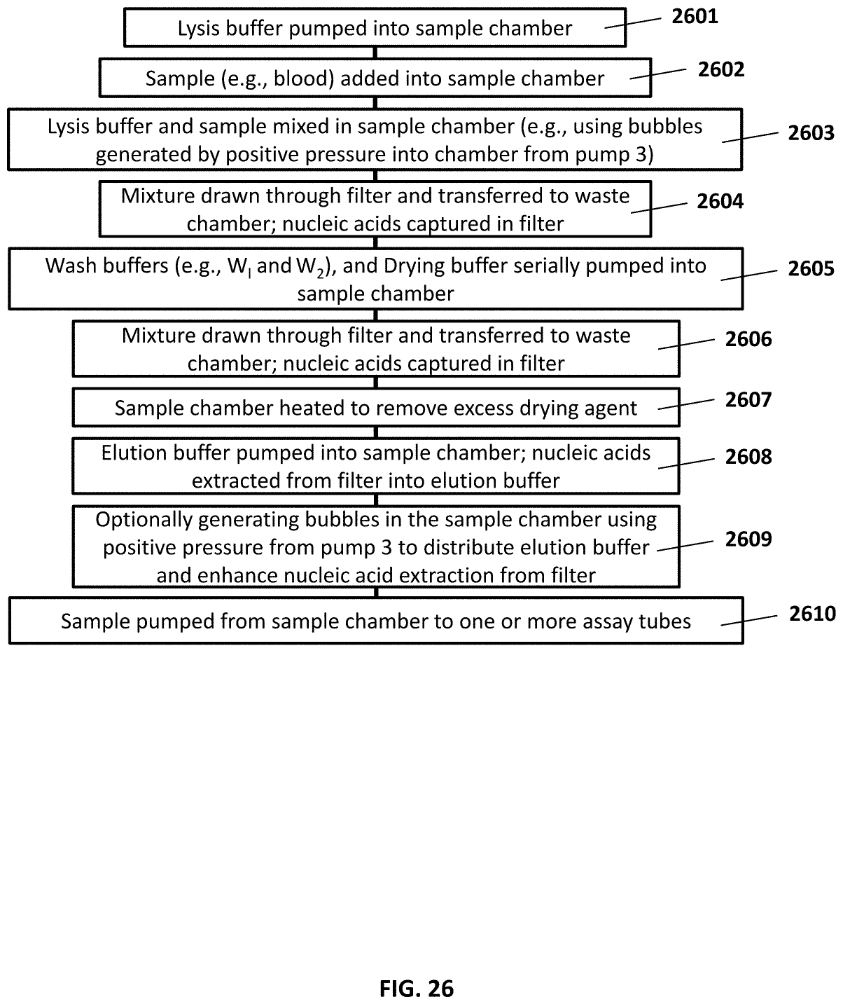

FIG. 26 shows an example process flow for using the system of FIGS. 22A-22E. In a first operation 601, a valve 2205 is opened and lysis buffer is pumped from a reagent chamber 2201 into the sample chamber 2204. In a second operation 602, a sample to be analyzed is added into the sample chamber 2204 now containing the lysis buffer. Filling the sample chamber 2204 with a buffer (e.g., a lysis buffer) prior to adding the sample may prevent loss of target nucleic acids within the sample (e.g., due to adhesion along the wall of the sample chamber). In a third operation 603, the lysis buffer and the sample are mixed in sample chamber 2204. The mixing can be performed in a variety of ways. In an example, bubbles can be generated by positive pressure into the sample chamber from a pump (e.g., first pump 2203, second pump 2208, or third pump 2209). Although any pump of the device may be used to generate bubbles within the sample chamber 2204, the pump 2209 may be used to avoid situations in which reversing the flow of the second pump 2208 (e.g., the waste pump), for example, may increase the risk of contamination of the sample in the sample chamber 2204 with waste from the waste chamber 2206. Other techniques may also be used to mix lysis buffer and sample in the sample chamber 2204, such as agitating the chamber 2201 or the entire device.

In a subsequent operation 604 the mixture of sample and lysis buffer is drawn through a filter 2302 by the second pump 2208, thereby capturing targets (e.g., nucleic acids) in the filter 2302 and transferring waste to a waste chamber 2206. In a subsequent operation 605, one or more wash buffers and/or drying buffers are serially pumped into sample chamber 2204, and mixed with the targets captured in the filter 2302. Subsequently, in operation 606, the mixture of buffer and target is drawn through the filter 2302 by pump 2208, thereby capturing targets (e.g., nucleic acids) in the filter 2302 and transferring waste to a waste chamber 2206. In some cases, in operation 607, following washing of the targets captured in the filter 2302 with a drying buffer (e.g., a volatile chemical such as acetone), the sample chamber may be heated (e.g., using a heating pad disposed along an outer surface of the sample chamber) to remove residual drying buffer (e.g., through vaporization). This may reduce contamination of the target by the drying agent. In a subsequent operation 608, elution buffer is pumped into sample chamber, thereby extracting a target (e.g., nucleic acids) from the filter into the elution buffer. In another operation 609, bubbles can be generated by positive pressure into the sample chamber from a pump to distribute the elution buffer throughout the sample chamber, and enhance extraction of the target from the filter. In yet another operation 610, the mixture of elution buffer and target is pumped by the third pump 2209 from the sample chamber 2204 to one or more assay tubes 2207 for further processing and/or analysis.

### Sample Preparation Cartridges

The present disclosure also provides sample preparation cartridges. Generally, sample preparation cartridges can comprise (i) one or more wells, each well containing a reagent necessary for processing the sample, (ii) a sample chamber for reacting the buffers with a sample, (iii) a chamber for depositing waste from the sample chamber, and (iv) one or more assay tubes for collecting a processed sample and performing an assay. Generally, the chambers and assay tubes can be connected by conduits (e.g., connections capable of transferring fluid from one chamber to another). Any of these conduits can comprise openings for connecting with a pump or valve to regulate flow of a liquid (e.g., a buffer or a sample) along the conduit.

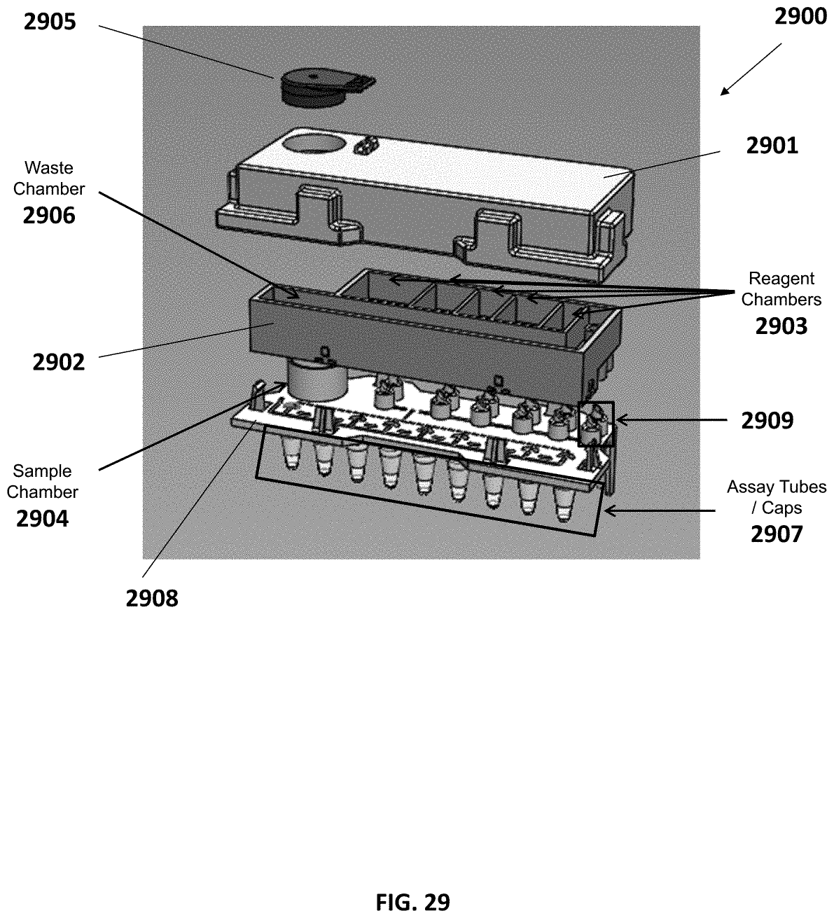

FIG. 29 shows an example of a sample preparation cartridge 2900. The sample preparation cartridge 2900 comprises a first manifold 2901 and a second manifold 2902. The second manifold 2902 comprises reagent chambers 2903 and a waste chamber 2906. The cartridge 2900 further comprises a third manifold 2908 comprising assay tubes 2907 and sample chamber 2904. The third manifold 2908 can comprise a plurality of needles 2909 which can be used to pierce the seal (e.g., foil) of reagent chambers to access the reagents. The needles can be hollow and can be connected to conduits for reagent transfers between different chambers. The first manifold 2901 can be a shroud (e.g., a cover). The cartridge 2900 also comprises a cap 2905. The cartridge 2900 may be used with methods and systems of the present disclosure.

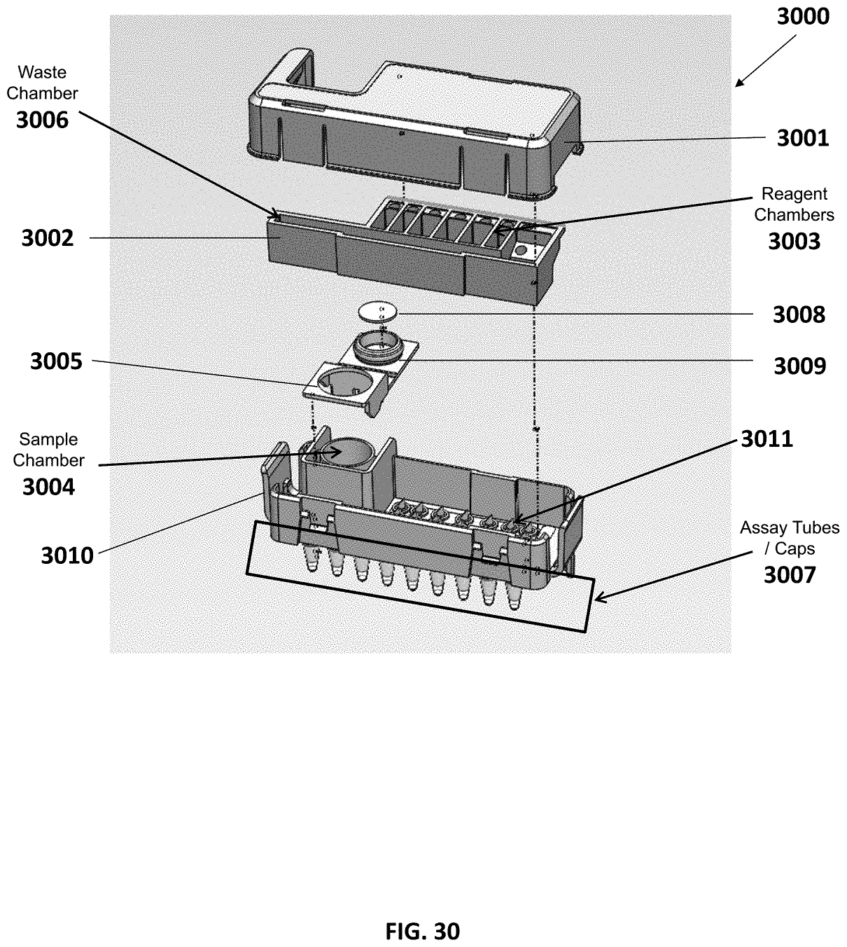

FIG. 30 shows another example of a sample preparation cartridge 3000. The sample preparation cartridge 3000 comprises a first manifold 3001 and a second manifold 3002. The second manifold 3002 comprises reagent chambers 3003 and a waste chamber 3006. The cartridge 3000 further comprises a third manifold 3010 comprising assay tubes 3007 and sample chamber 3004. The third manifold 3010 can comprise a plurality of needles 3011 which can be used to pierce the seal (e.g., foil) of reagent chambers to access the reagents. The needles can be hollow and can be connected to conduits for reagent transfers between different chambers. The first manifold 3001 can be a shroud. The cartridge 3000 also comprises an additional cover piece 3005 for the sample chamber 3004. The additional cover piece 3005 further comprises a folding rubber cap 3009. The folding rubber cap 3009 further comprises a porous disc 3008 which can prevent fluids and aerosols from escaping but allow air to pass through the folding rubber cap.

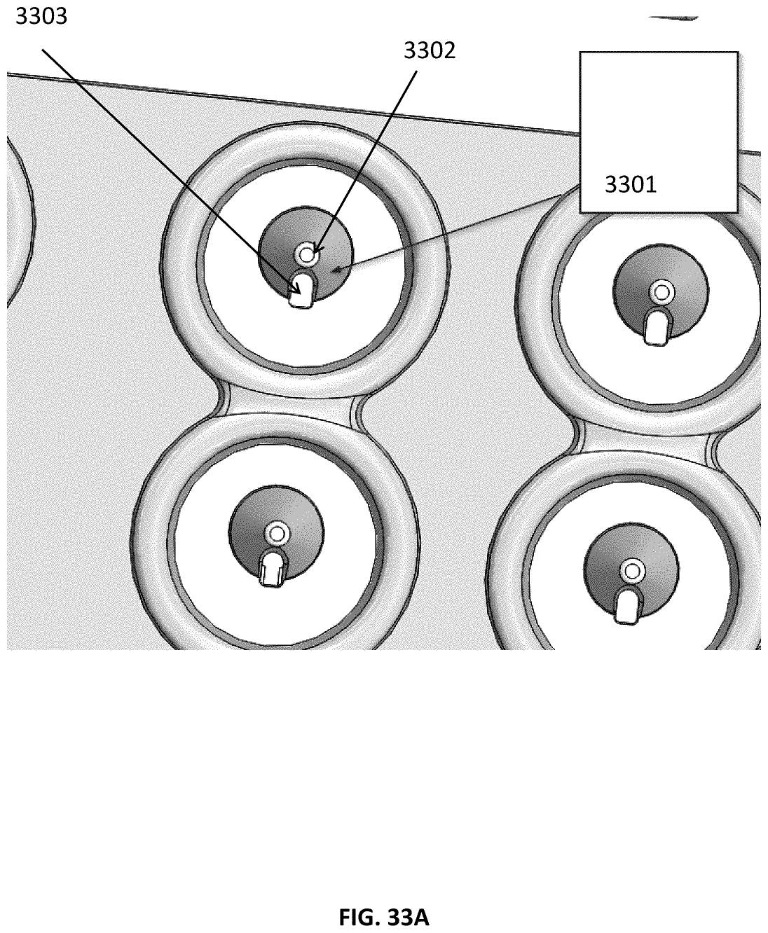

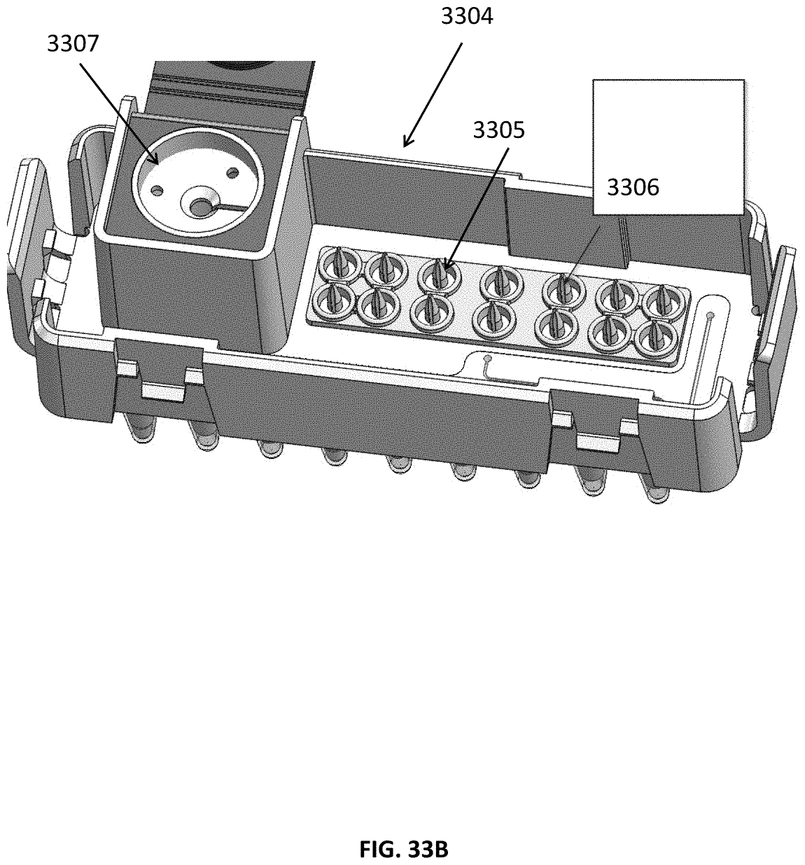

The needles used to pierce seals of reagent chambers can comprise one or more grooves. The one or more grooves can be used to drain reagents from the reagent chambers. The one or more grooves can prevent clogging or sealing of the needles when piercing the seals of the reagent chambers. FIG. 33A shows a top view of an example needle configuration. In this example, the needle 3301 comprises a hollow center 3302 and an off-centered groove 3303. FIG. 33B shows a sample preparation cartridge manifold 3304 having a plurality of needles 3305. Each needle within the manifold 3304 comprises a groove 3306. This needle configuration may allow the stiff small plastic needle with groove to prevent sealing during piercing of foil in fluid buffer tank (e.g., reagent chamber). The manifold 3304 comprises a sample chamber 3307.

### Materials

Sample preparation cartridges may be formed of various materials. In some cases, the sample preparation cartridge may be formed of a single material (e.g., polypropylene). In some cases, the sample preparation cartridge may be formed of two or more materials. In some cases, materials that are useful for producing sample preparation cartridges include materials suitable for three-dimensional (3D) printing, injection molding, or other methods capable of forming a device with three-dimensional compartments and/or embedded conduits for fluid transfer between compartments. Non-limiting examples of materials that may be used to produce the sample preparation cartridge include polysiloxane, polyphosphazene, low-density polyethylene (ldpe), high-density polyethylene (hdpe), polypropylene (pp), polyvinyl chloride (pvc), polystyrene (ps), nylon, nylon 6, nylon 6,6, teflon (polytetrafluoroethylene), thermoplastic polyurethanes (tpu), polychlorotrifluoroethylene (pctfe), bakelite, kevlar, twaron, mylar, neoprene, nylon, nomex, orlon, rilsan, technora, teflon, ultem, vectran, viton, zylon, polyamides, polycarbonate, polyester, polyethylene, polyvinylidene chloride (pvdc), acrylonitrile butadiene styrene (abs), polyepoxide, polymethyl methacrylate, maleimide, polyetherimide, polylactic acid, furan, silicone, or polysulfone. In some cases, the sample preparation cartridge can be formed of a material comprising a thermoplastic, a thermosetting polymer, an amorphous plastic, a crystalline plastic, a conductive polymer, a biodegradable plastic, or a bioplastic. In one example, a sample preparation cartridge may be formed of a material comprising polypropylene. In another example, a sample preparation cartridge may be formed of a first material comprising polypropylene and a second material comprising polycarbonate.

### Chambers

In some aspects, a sample preparation cartridge can comprise one or more chambers. Chambers may be useful for (i) storing buffers/reagents for sample processing, (ii) serially mixing a sample with a buffer or reagent to process a sample, and (iii) storing waste.

In some embodiments, a sample preparation cartridge can comprise 1 chamber. In some embodiments, a sample preparation cartridge can comprise a plurality of chambers. In some embodiments, a sample preparation cartridge can comprise 2 chambers, three chambers, 4 chambers, 5 chambers, 6 chambers, 7 chambers, 8 chambers, 9 chambers, 10 chambers, 15 chambers, 20 chambers, 25 chambers, 30 chambers, 35 chambers, 40 chambers, 45 chambers, 50 chambers, 100 chambers, or greater than 100 chambers. In one example, a sample preparation cartridge can comprise 5 chambers.

A size of a chamber (e.g., a sample chamber, a buffer chamber, or a waste chamber) can vary. In some embodiments, a chamber can hold at least about 0.1 milliliter (mL) of fluid. In some embodiments, a chamber can hold at least about 0.2 mL of fluid. In some embodiments, a chamber can hold at least about 0.3 mL of fluid. In some embodiments, a chamber can hold at least about 0.4 mL of fluid. In some embodiments, a chamber can hold at least about 0.5 mL of fluid. In some embodiments, a chamber can hold at least about 0.6 mL of fluid. In some embodiments, a chamber can hold at least about 0.7 mL of fluid. In some embodiments, a chamber can hold at least about 0.8 mL of fluid. In some embodiments, a chamber can hold at least about 0.9 mL of fluid. In some embodiments, a chamber can hold at least about 1 mL of fluid. In some embodiments, a chamber can hold at least about 1 mL, about 2 mL, about 3 mL, about 4 mL, about 5 mL, about 6 mL, about 7 mL, about 8 mL, about 9 mL, about 10 mL, or more of a fluid, such as a liquid.

In some embodiments, one or more of the chambers may be sealed. In some embodiments, the seal may be removable or breakable (e.g., a user may break the seal on the chamber to add a sample to the chamber). The seal may be formed of a single material (e.g., aluminum) or a composition of two or more materials. In one example, the sample preparation cartridge may be formed of a material comprising polypropylene, and the seal may be formed of a material that comprises a tri-layer of an aluminum, adhesive layer and polypropylene layer. In some cases the seal material may allow a plastic syringe to penetrate the seal. The seal material may be a foil laminate. In some cases, a seal may adhere to the sample preparation cartridge at temperatures of a minimum of 10° C. up to and including 54° C., and maintain a seal for at least about 1 month, at least about 6 months, at least about 12 months, at least about 24 months, at least about 36 months, at least about 48 months or at least about 60 months. In some cases, a chamber may be permanently sealed. For example, a sample preparation cartridge can comprise a waste chamber, and the waste chamber may be permanently sealed.

In some embodiments, one or more of the chambers may be covered by a shroud. For example, as shown in FIG. 29 and FIG. 30, the manifold having one or more chambers is covered by a shroud.

In some embodiments, a chamber can comprise a reagent for performing an assay (e.g., a lysis buffer, a wash buffer, a drying agent, or an elution buffer). Non-limiting examples of buffers can comprise NP-40 lysis buffer, Radio Immunoprecipitation Assay (RIPA) lysis buffer, sodium dodecyl sulfate (SDS) lysis buffer, Ammonium-Chloride-Potassium (ACK) lysing buffer, volatile chemicals (e.g., acetone and ethanol), EDTA, Tris-HCl, and water.

In some embodiments, a chamber can comprise one or more buffers useful for analyzing a sample according to the Boom Method. In accordance with the Boom method, a biological sample is lysed and/or homogenized by mixing the biological sample with detergent in the presence of protein degrading enzymes. The chaotropic agents and silica or silica coated beads are mixed with the lysed biological sample. The chaotropic agents disrupt and denature the structure of nucleic acids by interfering with the macromolecular interactions mediated by non-covalent forces, such as hydrogen bonding, van der Waals forces, and hydrophobic interactions, for example. In the presence of the chaotropic agents, water is removed from the phosphate groups of the nucleic acids, exposing them and allowing hydrophobic bonding to the silica, such as silica or silica coated beads. Protein, cellular debris, and other substances in the biological samples do not bond to the silica and are retained in the solution. The silica beads are washed several times to remove non-nucleic acid materials, such as proteins, lipids, cellular constituents, including cellular molecules, and other substances found in biological samples. Silica coated magnetic beads may be used to assist in the separation of the nucleic acids bound to the silica coating from the solution, via a magnetic field or magnet. The nucleic acids are then eluted from the silica or silica coated beads into a buffer by decreasing the concentration of the chaotropic agents. The elution buffer may be pure water or Tris-EDTA (“TE”) buffer, for example.

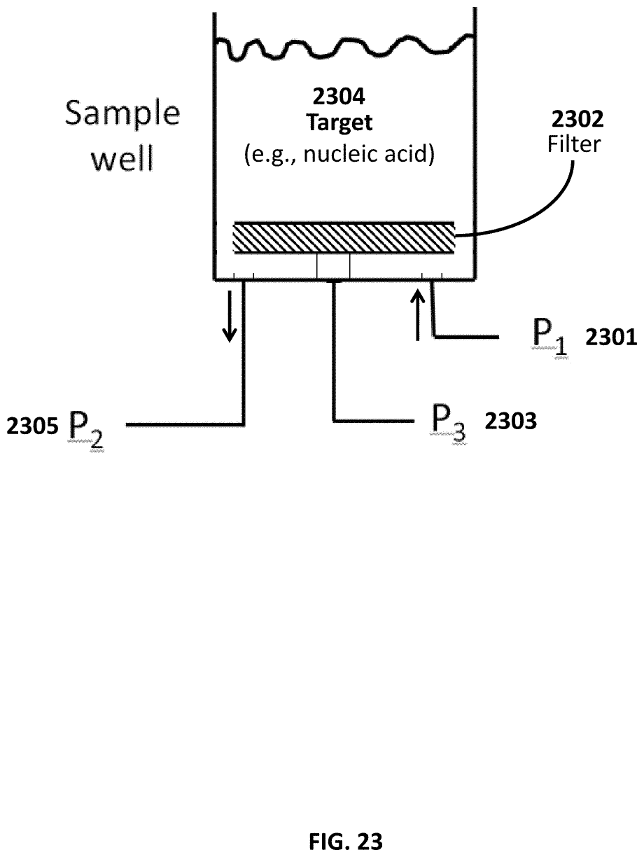

In some aspects, the sample preparation cartridge can comprise a sample chamber. Generally, a sample may be added to the sample chamber, after which buffers are serially added to the sample chamber to process the sample. A sample chamber may be fluidly connected to a buffer chamber (e.g., via a conduit) such that a pump disposed along the conduit can transfer the buffer automatically to the sample chamber. After mixing the sample with a given buffer, the mixture is pulled through a filter located within the sample chamber, the filter configured to capture a target (e.g., a nucleic acid) within the sample. An elution buffer may be added to the sample chamber to release the target from the filter. The sample chamber and filter can be configured such that fluid can be quickly pumped into the sample chamber (e.g., around the filter) and pumped out of the sample chamber through the filter (e.g., to capture a target in the sample). In some cases the filter may be movable (e.g., shift between a first position and a second position) to allow the fluid to quickly enter the sample chamber. In some cases, the filter may be capable of bending or translocating, e.g., as described in U.S. Pat. No. 9,926,553, which is entirely incorporated herein by reference. An example sample chamber is shown in FIG. 23. Reagents (e.g., lysis buffer, wash buffer) may be pump into the sample chamber 2304 using pressure generated from a pump 2301. In a filling stage (e.g., when reagent is being added to the sample chamber), a reagent may enter the sample chamber by flowing around a filter 2302. This can reduce the resistance experienced by the pump, and allow the reagent to fill the sample chamber more quickly. After the reagent has mixed with the sample, an additional pump 2303 may be used to transfer the mixture through the filter configured to capture a target (e.g., nucleic acid) 2304 in the sample to the waste chamber. After the target is processed and bound to the filter, an elution buffer may be pumped into the sample chamber to capture the target from the filter; a third pump 2305 may be used to transfer the sample to an assay tube for further analysis. In some embodiments, the sample chamber is covered by a cap. In some embodiments, the sample chamber is covered by a folding rubber cap. In some embodiments, the folding rubber cap comprises a porous disc. The porous disc can prevent fluids and aerosols from escaping but allow air to pass through the cap.

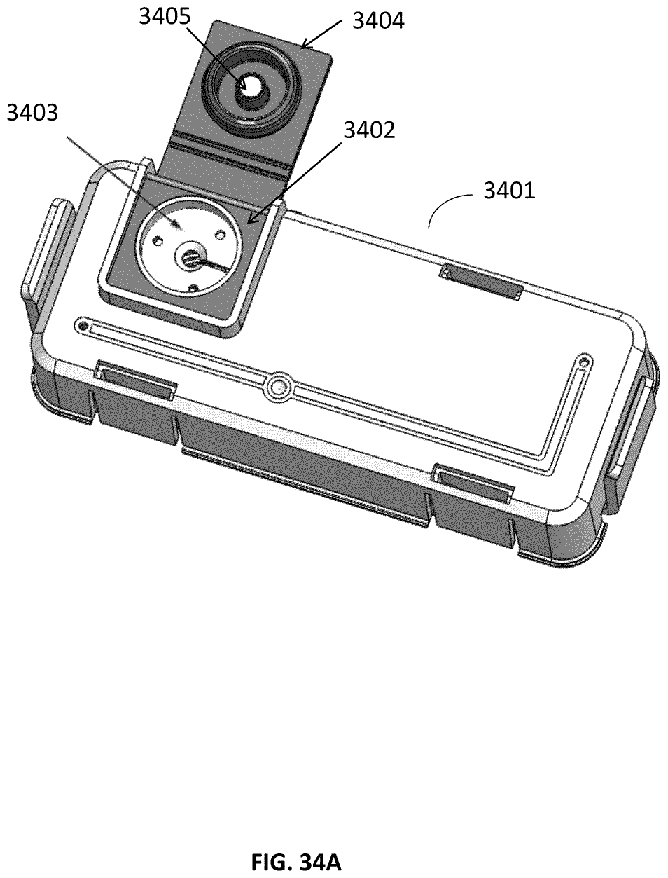

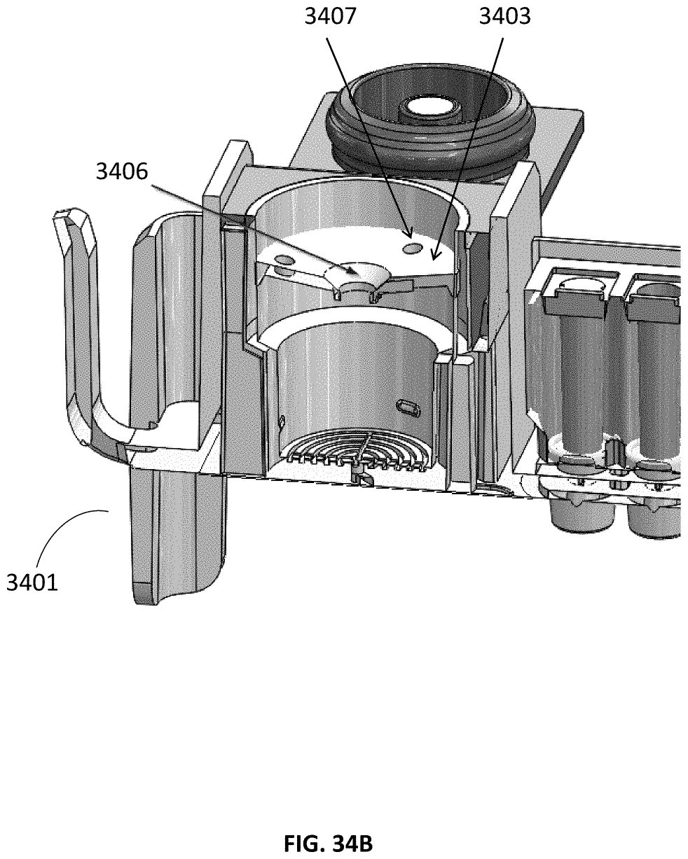

The sample chamber may further comprise a funnel. The funnel can allow liquid reagent to flow through during sample preparation, but can prevent sample loss (e.g., pellet loss) or fluid splashing onto the cap. In some cases, the funnel can prevent transfer of a sample from within the sample chamber to an external environment. FIG. 34A shows an example sample preparation cartridge 3401 having a funnel 3403 inserted into a sample chamber 3402. The sample chamber 3402 further comprises a cap 3404 having a vent plug 3405. In this example, the funnel 3403 can control sample pellet and prevent fluid splashing into the vent plug 3405 of the cap 3404. FIG. 34B shows a cross-section view of the funnel 3403 within the sample preparation cartridge 3401 shown in FIG. 34A. Sample liquid can go through the hole 3406 in the center of the funnel 3403. The funnel 3403 can further comprise periphery relief holes 3407.

The sample preparation cartridge can comprise a cap for the sample chamber. The cap be connected or disconnected to the sample chamber. The cap can comprise a vent plug. In some cases, a reagent chamber or waste chamber may also comprise a vent plug. The vent plug can be a self-sealing vent plug. For example, FIG. 35 shows an example sample preparation cartridge 3501 having one or more vent plugs installed. The sample preparation cartridge 3501 comprises a sample chamber 3505 connected to a cap 3504 having a vent plug 3502. The sample preparation cartridge 3501 also comprises a waste chamber 3506 having a vent plug 3503. The vent plug can swell when liquid contacts the vent plug to seal the chamber and can prevent escape or leak of hazardous material.

In some embodiments, it may be beneficial to heat the sample. Accordingly, a heater may be provided adjacent to the sample chamber (e.g., below the sample chamber) to provide heat to the sample and/or the sample chamber. For example, prior to extracting the target from the filter, the sample chamber and/or filter may be washed with a volatile solvent (e.g., ethanol or acetone). Subsequently, a heater may be used to apply heat to the sample and/or sample chamber to evaporate any remaining volatile solvent. It is also possible to improve both product yield as well as specificity of PCR by preparing a sample or a reaction mixture at increased temperatures (e.g., a temperature greater than an annealing temperature of a primer). Pre-amplification heating may promote annealing of the primer to a target nucleic acid, subsequent extension, as well as minimize the formation of primer-dimers or primer self-annealing. A pre-amplification heating step may be particularly useful for processing samples with low nucleic acid content, as the sample may be split into two or more assay tubes and pre-amplification heating of the sample may increase product yield in each assay tube. Accordingly, a pre-amplification heating step may be implemented in any of the embodiments of the present disclosure. For example, prior to transferring a sample from the sample chamber to one or more assay tubes, a heater may be used to heat the sample. In another example, lysis buffer may be pumped into the sample chamber, and subsequently heated. Heat can help denature the sample, reduce the formation of precipitates from the sample, or help return precipitated solids back into the sample solution. Using heat to homogenize the solution (e.g., reduce the precipitation of solids from the sample) can reduce buildup and clogs within a conduit as the sample is being transferred through the conduit. A heating step may be performed at any given temperature for any period of time. In some embodiments, a sample may be heated at 70° C. In some embodiments, a sample may be heated at 70° C. for a period of 10 minutes. In some embodiments, a sample may be heated at 70° C. indefinitely until the sample is transferred to one or more assay tubes for further processing. In some embodiments, a sample may be heated at a single temperature. In some embodiments, a sample may be heated over a range of temperatures (e.g., a range of increasing temperatures, or a range of decreasing temperatures). The heater may further comprise a spring-loaded plate. The spring-loaded plate can provide improved thermal contact with the sample chamber compared with a heater without such spring-loaded plate.

In general, a filter can comprise any material capable of capturing a target (e.g., a nucleic acid) from a sample. A filter may be organic or inorganic; may be metal (e.g., copper or silver) or non-metal; may be a polymer or may not be a polymer; may be conducting, semiconducting or nonconducting (insulating); may be reflecting or nonreflecting; may be porous or nonporous; etc. A filter as described above can be formed of any suitable material, including metals, metal oxides, semiconductors, polymers (particularly organic polymers in any suitable form including woven, nonwoven, molded, extruded, cast, etc.), silicon, silicon oxide, and composites thereof. A number of materials (e.g., polymers) suitable for use as filters in the instant invention may be used. Suitable materials for use as filters include, but are not limited to, polycarbonate, gold, silicon, silicon oxide, silicon oxynitride, indium, tantalum oxide, niobium oxide, titanium, titanium oxide, platinum, iridium, indium tin oxide, diamond or diamond-like film, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polyacrylonitrile (PAN), polyacrylate, polycarbonate, polybutylene terephthalate (PBT), poly(ethylene) (PE) (including low density, linear low density, high density, cross-linked and ultra-high molecular weight grades), poly(propylene) (PP), cis and trans isomers of poly(butadiene) (PB), cis and trans isomers of poly(isoprene), polyethylene terephthalate) (PET), polypropylene homopolymer, polypropylene copolymers, polystyrene (PS) (including general purpose and high impact grades), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), poly(styrene-acrylonitrile) (SAN), polytetrafluoroethylene (PTFE) and its derivatives, polyolefin plastomers, fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), styrene maleic anhydride (SMA), metal oxides, glass, glass wool, silicon oxide or other inorganic or semiconductor material (e.g., silicon nitride), compound semiconductors (e.g., gallium arsenide, and indium gallium arsenide), and combinations thereof.

Examples of filters include polypropylene, polystyrene, polyethylene, dextran, nylon, amylases, glass, natural and modified celluloses (e.g., nitrocellulose), polyacrylamides, agaroses and magnetite. In some instances, the filter can be silica or glass because of its great chemical resistance against solvents, its mechanical stability, its low intrinsic fluorescence properties, and its flexibility of being readily functionalized. In an example, the filter is formed of silicon oxide (e.g., glass).

A filter material may be modified with one or more different layers of compounds or coatings that serve to modify the properties of the surface in a desirable manner. For example, a filter may further comprise a coating material on the whole or a portion of the surface of the filter. For example, the coating material can be nitrocellulose, silane, thiol, disulfide, or a polymer. When the material is a thiol, the filter may comprise a gold-coated surface and/or the thiol comprises hydrophobic and hydrophilic moieties. When the coating material is a silane, the filter comprises glass and the silane may present terminal moieties including, for example, hydroxyl, carboxyl, phosphate, glycidoxy, sulfonate, isocyanato, thiol, or amino groups. In an alternative embodiment, the coating material may be a derivatized monolayer or multilayer having covalently bonded linker moieties. For example, the monolayer coating may have thiol (e.g., a thioalkyl selected from the group consisting of a thioalkyl acid (e.g., 16-mercaptohexadecanoic acid), thioalkyl alcohol, thioalkyl amine, and halogen containing thioalkyl compound), disulfide or silane groups that produce a chemical or physicochemical bonding to the filter. The attachment of the monolayer to the filter may also be achieved by non-covalent interactions or by covalent reactions.

After attachment to the filter, the coating may comprise at least one functional group. Examples of functional groups on the monolayer coating include, but are not limited to, carboxyl, isocyanate, halogen, amine or hydroxyl groups. In one embodiment, these reactive functional groups on the coating may be activated by standard chemical techniques to corresponding activated functional groups on the monolayer coating (e.g., conversion of carboxyl groups to anhydrides or acid halides, etc.). Examples of activated functional groups of the coating on the filter for covalent coupling to terminal amino groups include anhydrides, N-hydroxysuccinimide esters or other common activated esters or acid halides, Examples of activated functional groups of the coating on the filter include anhydride derivatives for coupling with a terminal hydroxyl group; hydrazine derivatives for coupling onto oxidized sugar residues of the linker compound; or maleimide derivatives for covalent attachment to thiol groups of the linker compound. To produce a derivatized coating, at least one terminal carboxyl group on the coating can be activated to an anhydride group and then reacted, for example, with a linker compound. Alternatively, the functional groups on the coating may be reacted with a linker having activated functional groups (e.g., N-hydroxysuccinimide esters, acid halides, anhydrides, and isocyanates) for covalent coupling to reactive amino groups on the coating.

In some embodiments, the sample preparation cartridge can also comprise a waste chamber. In some cases, a waste chamber may be fluidly connected to a sample chamber (e.g., via a conduit) such that the sample may be drawn through a filter in the sample chamber, and transferred the waste to the waste chamber.

### Conduits

Any compartment of the sample preparation cartridge (e.g., a chamber or an assay tube) may be fluidly connected to one or more other compartments of the sample preparation cartridge by one or more conduits. The sample preparation cartridge may comprise 2, 3, 4, 5, 6, 7, 8, 9, 10, 11, 12, 13, 14, 15, 16, or more conduits. Generally, a conduit may be used to connect two compartments in order to allow a sample or reagent to pass between the two compartments. For example, the sample chamber may be fluidly connected to the waste chamber to allow fluid to be pumped from the sample chamber to the waste chamber.

The structure of the sample preparation cartridges described herein can comprise an aggregation of two or more separate layers which when appropriately mated or joined together, form the conduits described herein. For example, a bottom surface of a top layer and a top surface of a bottom layer can each comprise a trench (e.g., a channel or a groove) that, when mated together, form a conduit. Typically, the sample preparation cartridges described herein will comprise a top portion, a bottom portion, and an interior portion, wherein the interior portion substantially defines the conduits of the cartridge. For example, the body structure is fabricated from at least two substrate layers that are mated together to define the conduit networks of the cartridge, e.g., the interior portion. In some cases, the top portion of the cartridge can comprise the chambers (e.g., sample chambers, buffer chamber, and waste chamber). In some cases, the bottom portion of the cartridge comprises one or more adapters or caps to which an assay tube may be coupled.

A variety of materials may be employed to fabricate the top and/or bottom layer of the sample preparation cartridge, as described above. In some cases, materials can be selected based upon their compatibility with various fabrication techniques, e.g., photolithography, wet chemical etching, laser ablation, air abrasion techniques, LIGA, reactive ion etching (RIE), injection molding, embossing, and other techniques. The materials can also generally be selected for their compatibility with the full range of conditions to which the sample preparation cartridges may be exposed, including extremes of pH, temperature, salt concentration, and application of electric fields. Accordingly, in some aspects, the material may include, e.g., silica based substrates, such as glass, quartz, silicon or polysilicon. In the case of semi conductive materials, it will often be desirable to provide an insulating coating or layer, e.g., silicon oxide, over the material, and particularly in those applications where electric fields are to be applied to the cartridge or its contents.

In some aspects, the materials will comprise polymeric materials, e.g., plastics, such as polymethylmethacrylate (PMMA), polycarbonate, polytetrafluoroethylene (TEFLON™), polyvinylchloride (PVC), polydimethylsiloxane (PDMS), polysulfone, and the like. Such polymeric substrates may be readily manufactured using fabrication techniques; using molding techniques, such as injection molding, embossing or stamping; or by polymerizing the polymeric precursor material within the mold. Such polymeric materials are for their ease of manufacture, low cost and disposability, as well as their general inertness to most extreme reaction conditions. Again, these polymeric materials may include treated surfaces, e.g., derivatized or coated surfaces, to enhance their utility in the sample preparation cartridge, e.g., provide enhanced fluid direction.

Sample preparation cartridges may be used in a variety of applications, including, e.g., the performance of high throughput screening assays in drug discovery, immunoassays, diagnostics, nucleic acid analysis, including genetic analysis, and the like. As such, the cartridges described herein, will often include one or more conduit openings. A conduit opening can generally refer to any opening through which a conduit, and the corresponding chamber to which the conduit is connected, may be accessed. Conduit openings may be useful for a variety of reasons. Firstly, conduit openings can allow for the insertion of a pump or valve along the conduit. This is particularly useful for preparing disposable sample preparation cartridges, as described below. Conduit openings in the sample preparation cartridge allow the cartridge to dock with the re-useable sample preparation device (e.g., the re-useable device comprising pumps, valves, and/or electronic components). Conduit openings in the sample preparation cartridge allow for the cartridge to be produced without more expensive components.

Secondly, conduit openings can allow for different chambers of the sample preparation cartridge to be fluidly connected, via their respective conduits, depending on the assay being performed. In some embodiments, a sample preparation cartridge can comprise multiple sets of reagents, each set of reagents for processing a sample for a particular assay to be performed. For example, a sample preparation device may be configured such that, upon docking the sample preparation cartridge to the sample preparation device, the reagents in chambers 1 through 5 are serially transferred to the sample chamber. In another example, a sample preparation device may be configured such that, upon docking the sample preparation cartridge to the sample preparation device, the reagents in chambers 6 through 10 are serially transferred to the sample chamber. In yet another example, a sample preparation device may be configured such that, upon docking the sample preparation cartridge to the sample preparation device, the reagent in chamber 1 is mixed with the reagent in chamber 2, and subsequently the mixture of the two reagents is serially transferred to the sample chamber.

In some embodiments, a sample preparation cartridge can comprise a separate conduit that is fluidly connected to each chamber on the cartridge. In some embodiments, a sample preparation cartridge can comprise two or more chambers that are connected to a common or primary conduit via separate secondary conduits. For example, a first conduit fluidly connected to a first chamber and a second conduit fluidly connected to a second chamber may both fluidly connect to a primary conduit. Any number of secondary conduits, each of which may be fluidly connected to one a chamber or assay tube, maybe fluidly connected to a primary conduit. In some cases where multiple secondary conduits are fluidly connected to a single primary conduit, valves may be used to restrict flow to one or more specific secondary conduits.

Multiple sample introduction ports or sample chambers are contemplated for the parallel or serial introduction and analysis of multiple samples. Alternatively, cartridges may be coupled to a sample introduction port, e.g., a pipette, which serially introduces multiple samples into the cartridge for analysis.

### Assay Tubes and Caps

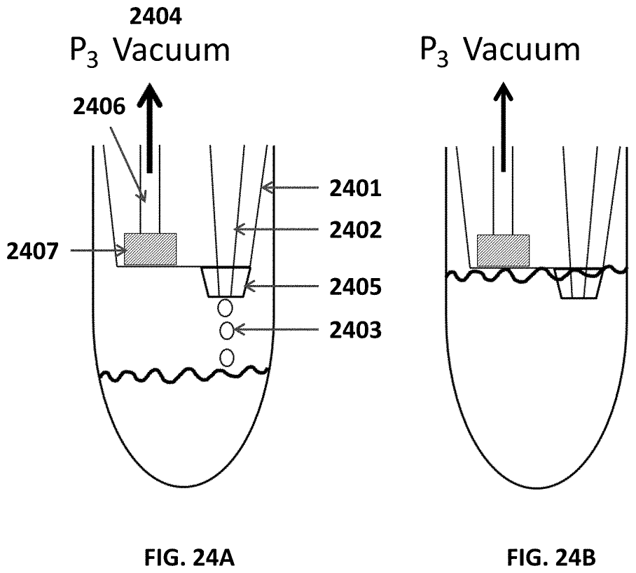

In some embodiments, a sample preparation cartridge of the present disclosure can comprise one or more assay tubes, each having an assay tube cap, fluidly connected to the sample chamber (see, e.g., FIGS. 24A and 24B). The assay tube cap can be part of a manifold of the sample preparation cartridge. The assay tube cap can be connected to one or more conduits of a manifold of the sample preparation cartridge. It should be understood that an assay tube may be interchanged with a chamber in any embodiment of the present disclosure. Generally, following processing of the sample, an elution buffer may be added to the sample chamber to extract the target (e.g., nucleic acid) from the filter, and transfer the target to the assay tube. Assay tubes may be transparent, such that they are capable of transmitting an optical signal from the sample in the assay tube, the optical signal capable of being detected by an analytic device. Various PCR tubes may be used. For example, the assay tube may be a 0.1 mL or 0.2 mL PCR tube, or other thin-walled commercially available PCR tubes. Suitable PCR tubes may be obtained from Phenix Research Products, Candler, N.C., BIOplastics, for example. In some embodiments, an assay tube cap may be removably coupled (e.g., separable) to the sample preparation cartridge; an assay tube may be coupled directly to the cap. For example, the sample preparation cartridge may be removably attached (e.g., by a perforation) to a strip of assay tube caps to which assay tubes may be press-fit or snap fit. Having one or more assay tube caps removably attached to the sample preparation cartridge can be advantageous as the assay tubes (containing a sample) and caps can be quickly separated from the sample preparation cartridge and loaded into an analytic device (e.g., a thermal cycler).

In some embodiments, an assay tube cap 2401 can comprise one or more conduits 2402 through which (i) a sample 2403 may be transferred into the assay tube, and/or (ii) a pressure 2404 may be applied (e.g., to draw fluid into the assay tube). Generally, a conduit may pass through the assay tube cap, thereby providing a fluid connection between the assay tube and the conduit (e.g., a conduit extending from a sample chamber).

An assay tube cap can have one or more first conduits (also referred to as inflow conduits) passing through the cap to supply the assay tube with a reagent or sample. In some embodiments, an end of the conduit can have a tip or nozzle 2405, to control the flow of a reagent or sample out of the conduit. A person having skill in the art will appreciate that a variety of different aspects of the flow may be controlled. Non-limiting examples include the flow rate, the type of flow (e.g., laminar or turbulent), and a size of droplet formed. Two concerns in liquid delivery through nozzles include (i) how to eject a droplet cleanly so that a drop is not left hanging on the end of the nozzle, and (ii) how to keep the contents of the assay tube from splashing when the stream of liquid is delivered into the assay tube. Further, the ejection velocity of the liquid from the nozzle may be sufficient to induce mixing between the first and second delivered liquid in the reaction chamber. Very small droplets can be ejected cleanly at high ejection velocities, but do not have sufficient kinetic energy to overcome the surface tension of the liquid already in the well to cause mixing. In contrast, larger droplets also eject cleanly at high ejection velocities, but tend to splash the contents into adjacent wells. At lower ejection velocities, the liquids tend to leave the last drop hanging from the nozzle tip, which is also a function of the cross-sectional area of the tip. Moreover, the flow rate of liquids through the conduit varies directly with the delivery pressure and inversely with the length of the conduit and inversely with the diameter. All these variables may be taken into consideration when developing delivery pressure and tip configurations, as well as the materials of construction, so that the liquids can be expelled cleanly without leaving a residual drop of liquid hanging from the nozzle tip. In some cases, the nozzle or tip may be used to increase a cross-sectional area of the conduit. In some cases, the cross-sectional area of the conduit may gradually increase along a length of the nozzle or tip. In some cases, the nozzle or tip may be used to decrease a cross-sectional area of the conduit. In some cases, the cross-sectional area of the conduit may gradually decrease along a length of the nozzle or tip. The nozzle can be any shape. In some embodiments, a nozzle may be conical in shape. In some cases, the nozzle may be cylindrical in shape. In some cases the nozzle may be hemispherical in shape. The shape of the nozzle may be selected based on depending on the liquid, it may be more beneficial to dispense it in a continuous stream, a series of pulses or in droplet form.

In some cases, an assay tube cap can have one or more second conduits 2406 passing through the cap. These one or more second conduits can be coupled to a pump, and used to generate a draw pressure through the assay tube to draw a sample or reagent from a chamber (e.g., the sample chamber) to the assay tube. It is contemplated that a hydrophobic and/or porous material 2407 may be use to prevent liquid from entering the second conduit as the sample fills the assay tube. For example, a molecular sieve (e.g., a material permeable to a gas but not liquid) may be positioned at an end of the second conduit such that a draw pressure can be applied through the sieve to draw a sample into the assay tube. The molecular sieve may be permeable to one or more gases, such as air. However, as the sample fills the assay tube (see, e.g., FIG. 24B), the molecular sieve may prevent the sample from flowing into the second conduit. A molecular sieve used in any embodiment of the present disclosure can be a microporous molecular sieve, a mesoporous molecular sieve, or a macroporous molecular sieve. Non-limiting examples of molecular sieves include zeolites, aluminosilicate materials, porous glass, active carbon, clay, monmorillonite, halloysite, silicon dioxide, and silica. In some cases, the molecular sieve is a filter, for example, a pipette tip filter. The filter can self-seal upon contacting a liquid. The filter material may be hydrophobic, for example, polytetrafluoroethylene and polyethylene. In some cases, the filter have a small pore size, for example, from 10 to 12 μm, from 12 to 15 μm, from 15 to 20 μm, or from 20 to 25 μm.

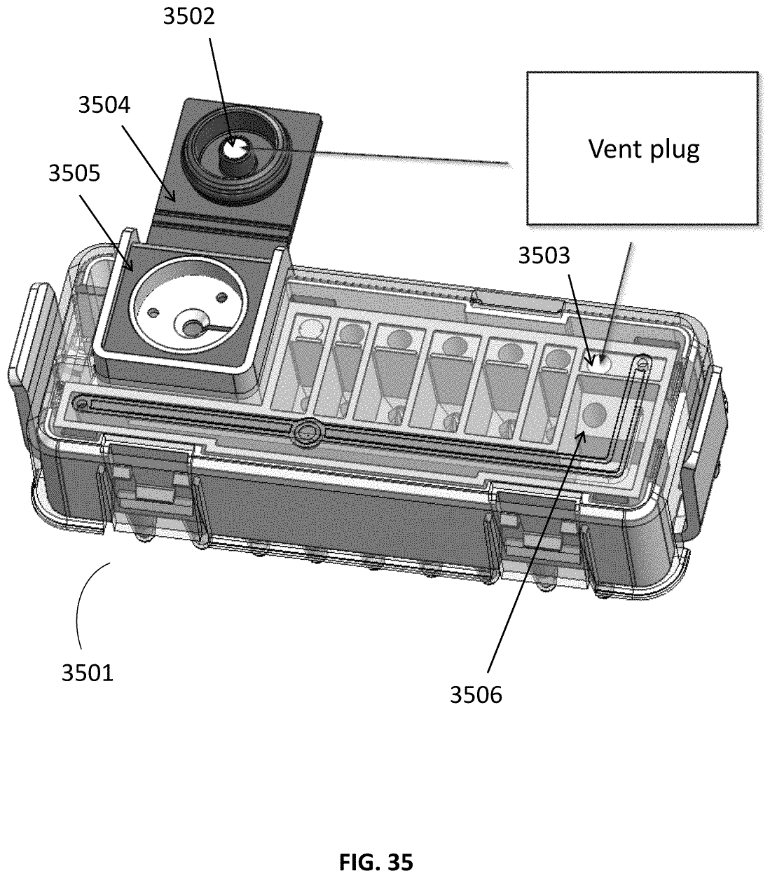

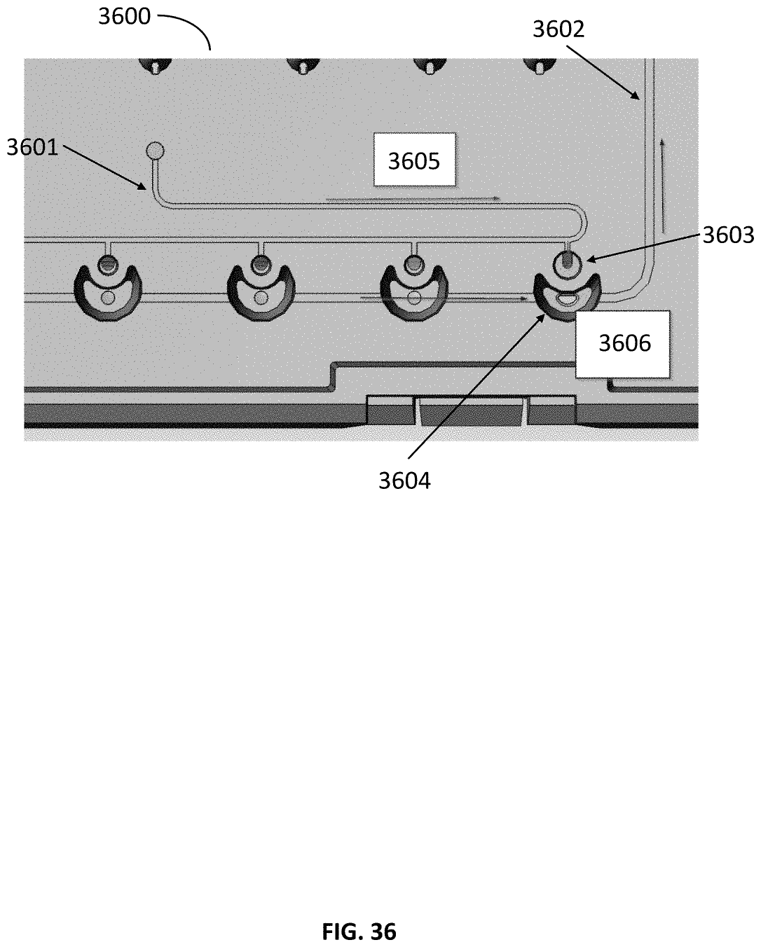

FIG. 36 shows a top view of a manifold 3600 of the sample preparation cartridge. The manifold comprises one or more conduits including one conduit 3601 in fluid communication with a first conduit 3603 passing through the cap to supply the assay tube with a reagent or sample and one conduit 3602 in connection with a second conduit 3604 passing through the cap to generate a draw pressure. The conduit 3602 or the second conduit 3604 passing through the cap can be coupled to a pump to generate the draw pressure or vacuum such that fluid (e.g., reagent or sample) can be drawn through the first conduit 3603 into the assay tube. Under ambient pressure, liquid analyte can flow from the sample chamber (not shown) into the conduit 3601 leading to the assay tube. The analyte can pass from this conduit into the assay tube via vertical conduit 3603. The arrow 3605 in parallel to the conduit 3601 indicates the direction of fluid flow into the assay tube. The arrow 3606 in parallel to the conduit 3602 indicates the direction of air flowing out from the assay tube.

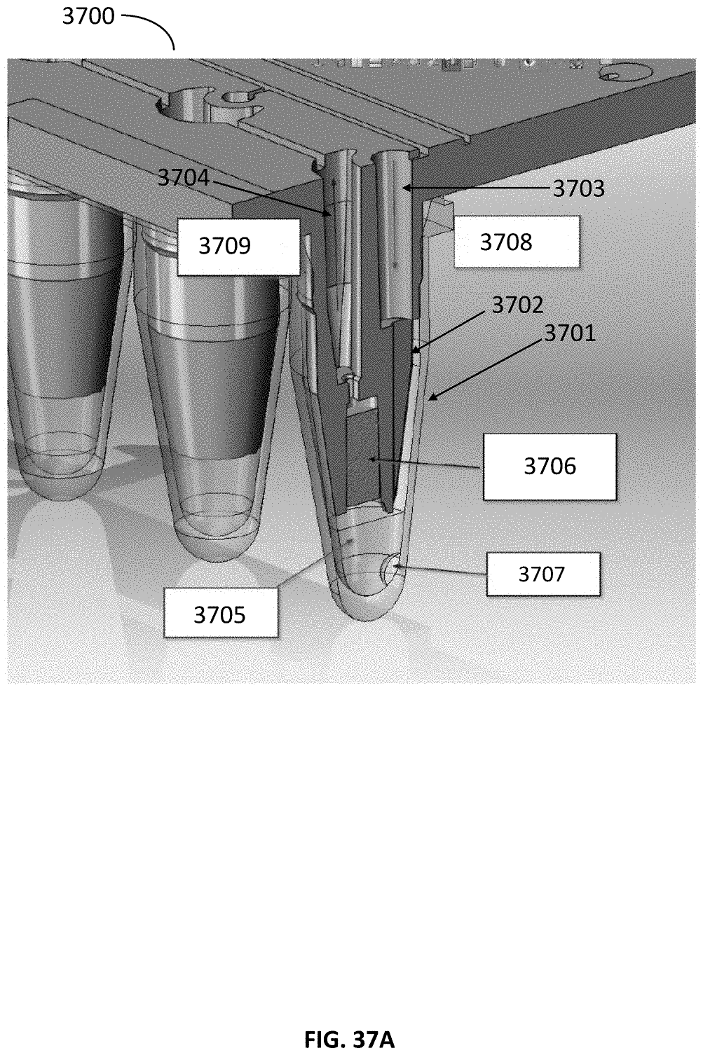

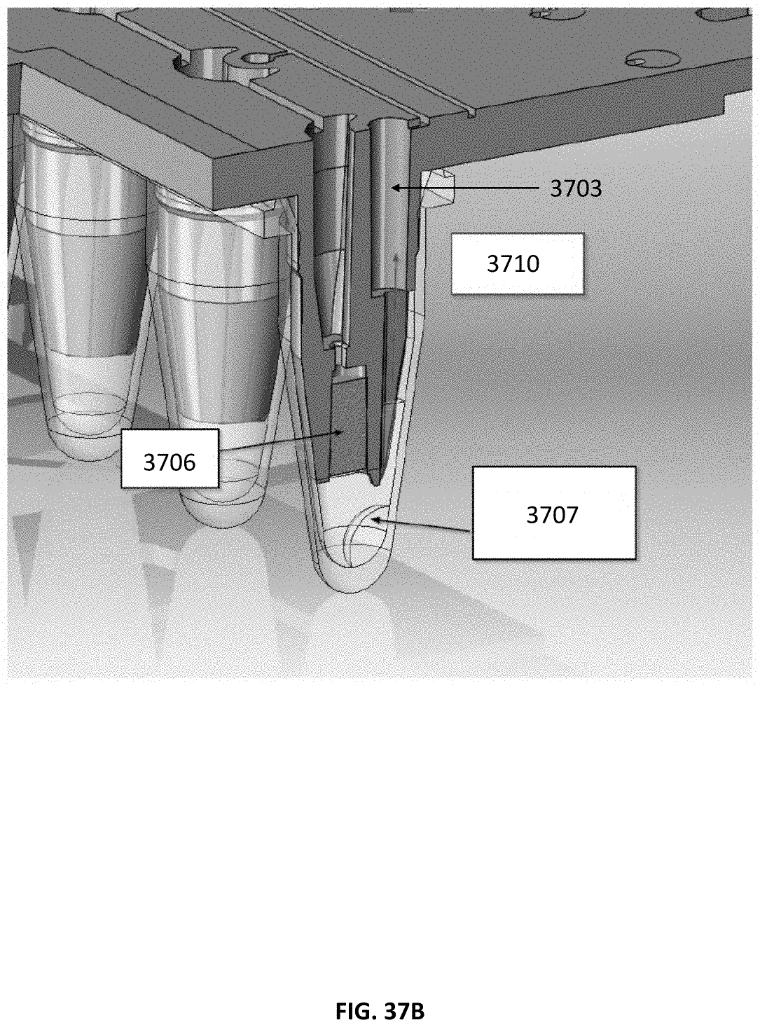

FIG. 37A shows a cross-sectional side view of the sample preparation cartridge of FIG. 36. The sample preparation cartridge can comprise a manifold 3700 having one or more conduits, one or more assay tubes 3701, and one or more assay caps 3702 inserted into the one or more assay tubes 3701. Under ambient pressure, liquid can flow from the sample chamber (not shown) into the conduit leading to the assay tube. The liquid can pass from this conduit into the assay tube via a first conduit 3703 (e.g., conduit vertical to the surface of the manifold) passing through the assay cap 3702. Liquid can fill the assay tube until the liquid level reaches a porous medium 3706 (e.g., a molecular sieve or a porous self-sealing filter medium) within the second conduit 3704 of the assay cap 3702. The porous medium 3706 can restrict liquid flow and allow gas to pass freely. The porous medium 3706 can be a porous plug or capillary. The arrow 3708 within the first conduit 3703 indicates the direction of fluid flow into the assay tube. The arrow 3709 within the second conduit 3704 indicates the direction of air flowing out from the assay tube. Upon contact with the fluid, the porous medium can swell and seal the conduit 3704 such that the connection to vacuum can be broken and fluid flow can halt, leaving a predetermined volume of liquid 3705 in the assay tube. This process may take a few seconds. However, during fill, air bubbles (or gas bubbles) 3707 may result from poor wetting of assay tube walls, voids in lyophilized reactant dried in the assay tube, or entrained air in the conduit leading to the assay tube (see, for example, the conduit 3703 in FIG. 37A). These air bubbles can have adverse effects. During fill, incoming liquid can solubilize lyophilized or freeze dried reactant located in the assay tube. Less liquid due to the air bubbles may increase reactant concentrations above the concentrations intended, which can affect PCR outcomes. Another problem caused by the air bubbles may arise during thermal cycling of the sample in the assay tube. FIG. 37B shows an example of the problem described herein. The air bubble 3707 may expand with increasing temperatures, displacing fluid back into the conduit leading to the assay tube (see, for example, the conduit 3703 in FIG. 37A and FIG. 37B). The arrow 3710 within the conduit 3703 indicates the direction of fluid flowing out from the assay tube due to expansion of the air bubble 3707. Upon cooling, contraction of air can cause the fluid volume to return. In some cases, the fluid may return to adjacent assay tubes. Such a problem can be referred to as “thermal pumping.” The results of the thermal pumping can include reduced thermal cycling efficiency, mixing of reactants from adjacent assay tubes, loss of fluids, and/or complete failure of PCR.

In an example of an approach to solve the thermal pumping problem, a valve or a seal may be used on the inflow conduit passing through the assay cap after fill. The valve or the seal can effectively trap air or gas in the fluid flowing in from the conduit and prevent temperature-driven volume displacement of fluid during thermal cycling. The valve may be a one-way valve. The valve may not be a one-way valve and can prevent fluid flow in either direction such that thermal pumping (e.g., liquid expansion or contraction during thermal cycling) can be prevented. The valve or seal may comprise a self-sealing or swellable material that can allow sufficient fluid to fill the assay tube before closing or sealing the inflow conduit after filling the fluid. The self-sealing or swellable property along with space constraints and the size of the features may make finding a valve or seal challenging. An approach may be to use a self-sealing or swellable particle (e.g., bead). The self-sealing or swellable particle can be disposable. The self-sealing or swellable particle can be single-use. The self-sealing or swellable particle can swell (or expand) when the particle is exposed to a liquid such as water. The self-sealing or swellable particle can be a gel particle (e.g., gel bead). The self-sealing or swellable particle can be a hydrogel particle or a hydrogel valve, which can be inexpensive and readily obtainable. The hydrogel particle can comprise a polymeric material (or a polymer). The polymeric material includes, but are not limited to, sodium polyacrylate, polyacrylamide, poly(ethylene glycol) and derivatives thereof (e.g. PEG-diacrylate (PEG-DA), PEG-RGD), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, and methylcellulose. The swellable particle can be obtained in precise geometries with appropriate swell rates under the conditions of fill. When the swellable particles are dry, they can be loaded into the inflow conduit passing through the assay cap such that there can be a gap between the outer surface of the particle and the inner wall of the inflow conduit. The gap can be at least about 0.1, 0.15, 0.2, 0.25, 0.3, 0.35, 0.4, 0.45, 0.5, 0.55, 0.6, 0.65, 0.7 or more millimeters (mm). The inflow conduit can have a size (e.g., diameter) of at least about 0.5, 0.6, 0.7, 0.8, 0.9, 1.0, 1.1, 1.2, 1.3, 1.4, 1.5, 1.6, 1.7, 1.8, 1.9, 2.0 or more mm. The dry swellable particle can have a size (e.g., diameter) of at least about 0.2, 0.25, 0.3, 0.35, 0.4, 0.45, 0.5, 0.55, 0.60, 0.65, 0.70, 0.75, 0.80, 0.85, 0.90, 1.0, 1.1, 1.2, 1.25, 1.3, 1.35, 1.4, 1.45, 1.5, 1.55, 1.6, 1.65, 1.7, 1.75, 1.8 or more mm. Upon contact with liquid when the fluid flows into the conduit, the swellable particle can begin to swell. The time-to-seal may be greater than the fill duration. The size of the channel or the swellable particle can be optimized to ensure fill, minimize seal lag (e.g., time between fill and seal) and/or maximize seal reliability (e.g., ensuring swell is adequate to seal).

The self-sealing or swellable particle may be self-sealing, swellable, or both self-sealing and swellable. The self-sealing or swellable particle may have various shapes, sizes and/or configurations. The self-sealing or swellable particle may have a shape that is circular, triangular, square, rectangular, pentagonal, hexagonal, or partial shapes or combinations of shapes thereof. The self-sealing or swellable particle may be spherical or non-spherical. The self-sealing or swellable particle may be a combination of smaller particles. The self-sealing or swellable particle may have a size (e.g., diameter) that is at least about 50, 100, 150, 200, 250, 300, 350, 400, 450, 500, 550, 600, 650, 700, 750, 800, 850, 900, 1,000, 1,200, 1,500, 2,000, 2,500, 2,800 or more microns. The self-sealing or swellable particle may have a size that is at most about 5,000, 4,500, 4,000, 3,500, 3,000, 2,500, 2,000, 1,500, 1,000 or less microns.

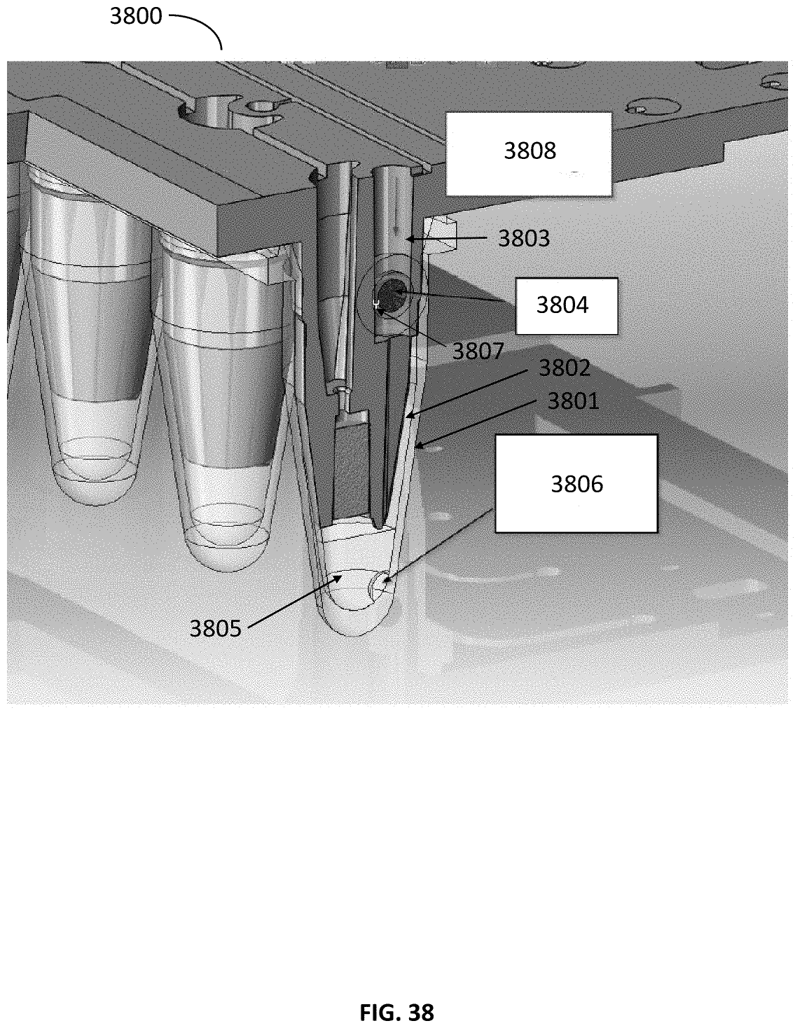

FIG. 38 shows an example configuration of the swellable particle loaded within the inflow conduit for sealing the conduit after liquid fill. The manifold 3800 of the sample preparation cartridge comprises one or more assay caps 3802 inserted into one or more assay tubes 3801. The assay cap 3802 comprises one or more conduits (or inflow conduits) 3803 for filling the assay tube 3801 with the liquid 3805. The conduit 3803 comprises a swellable particle 3804 loaded within the conduit. A gap 3807 is present between the outer surface of the swellable particle 3804 and the inner wall of the conduit 3803. An air bubble 3806 may be generated during the fill. The arrow 3808 within the conduit 3803 indicates the direction of fluid flow into the assay tube. The swellable particle 3804 can allow the fluid flowing into the assay tube 3801 through the conduit 3803, but can swell to seal the conduit upon filling the assay tube with the fluid. In this example configuration, the fill duration may be at least about 1, 2, 3, 4, 5, 6, or more seconds. Seal upon swelling of the swellable particle may be obtained at least about 3, 4, 5, 6, 7, 8, 9, 10, 11, 12 or more seconds after wetting (e.g., contacting with the fluid flowing into the assay tube). The swell rate of the swellable particle may be tunable by customizing the compositions or chemistry of the hydrogel. The swellable particle may swell to at least 2, 2.1, 2.2, 2.3, 2.4, 2.5, 2.6, 2.7, 2.8, 2.9, 3 or more times the size (e.g., diameter) of the dry particle.

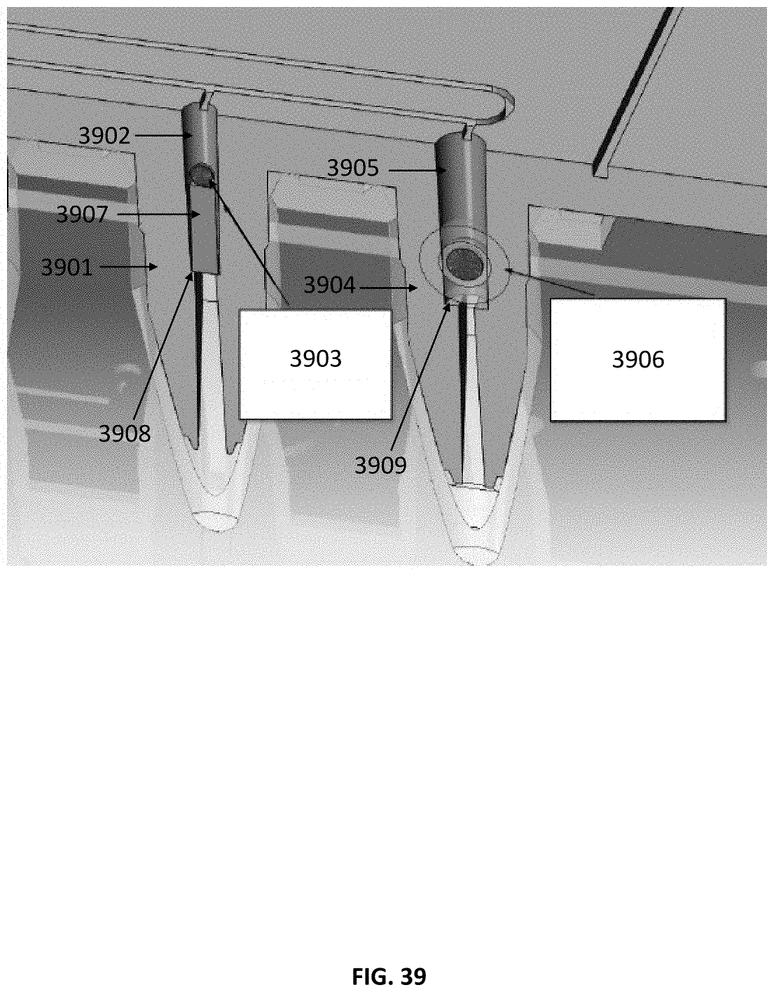

FIG. 39 shows two different example configurations of the inflow conduit loaded with the swellable particle. The left assay cap 3901 comprises an inflow conduit 3902 having a size (e.g., diameter) of about 1.1 mm. The dry swellable particle 3903 loaded within the inflow conduit 3902 can have a size (e.g., diameter) of at least about 0.60, 0.65, 0.70, 0.75, 0.80, 0.85, 0.90 or more mm. The right assay cap 3904 comprises an inflow conduit 3905 having a size (e.g., diameter) of about 1.60 mm. The dry swellable particle 3906 loaded within the inflow conduit 3905 can have a size (e.g., diameter) of at least about 0.80, 0.90, 1.0, 1.1, 1.2, 1.25, 1.3, 1.35, 1.4, 1.45, 1.5, 1.55, 1.6, 1.65, 1.7, 1.75, 1.8 or more mm. The inflow conduit can comprise an inner surface such as the inner surface 3908 of the inflow conduit 3902 and the inner surface 3909 of the inflow conduit 3905. The swellable particle can be supported by the inner surface. The inner surface may further comprise a support (e.g., the support 3907) in between the swellable particle and the inner surface of the inflow conduit. The support can be a plastic support such as a plastic rod. The plastic support can comprise polyamide, polycarbonate, polyester, polyethylene, polypropylene, polystyrene, polyurethanes, polyvinyl chloride, polyvinylidene chloride, acrylonitrile butadiene styrene, polytetrafluoroethylene (PTFE), or any combinations thereof.

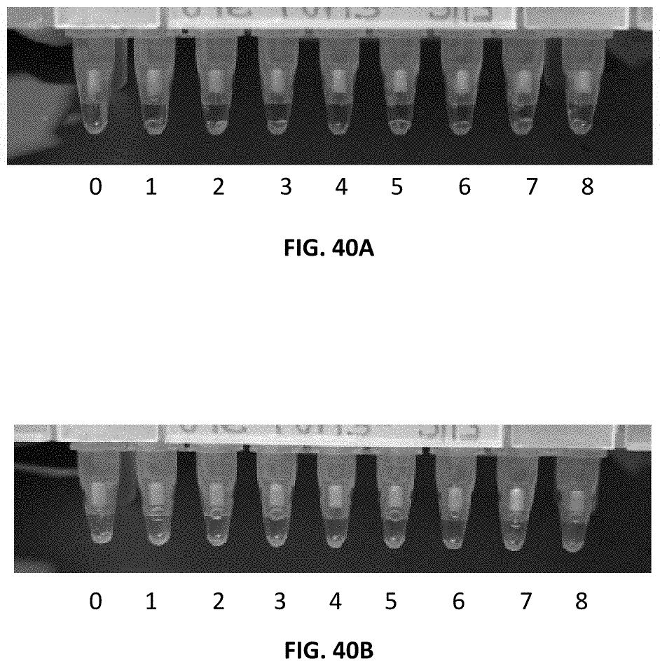

FIG. 40A shows an example sample preparation cartridge having an array of assay tubes filled with liquid samples with gas bubbles near bottom of the assay tubes. In this example, the sample preparation cartridge comprises swellable particles (e.g., hydrogel particles) within the inflow conduits. The numbers indicate well numbers. FIG. 40B shows an image of the same sample preparation cartridge of FIG. 40A after performing PCR or thermo-cycling. In this example, no fluid was displaced in the assay tubes. The gas bubbles remain in the assay tubes after thermal cycling. The gas bubbles did not increase in size but rose to the top of the fluid sample.

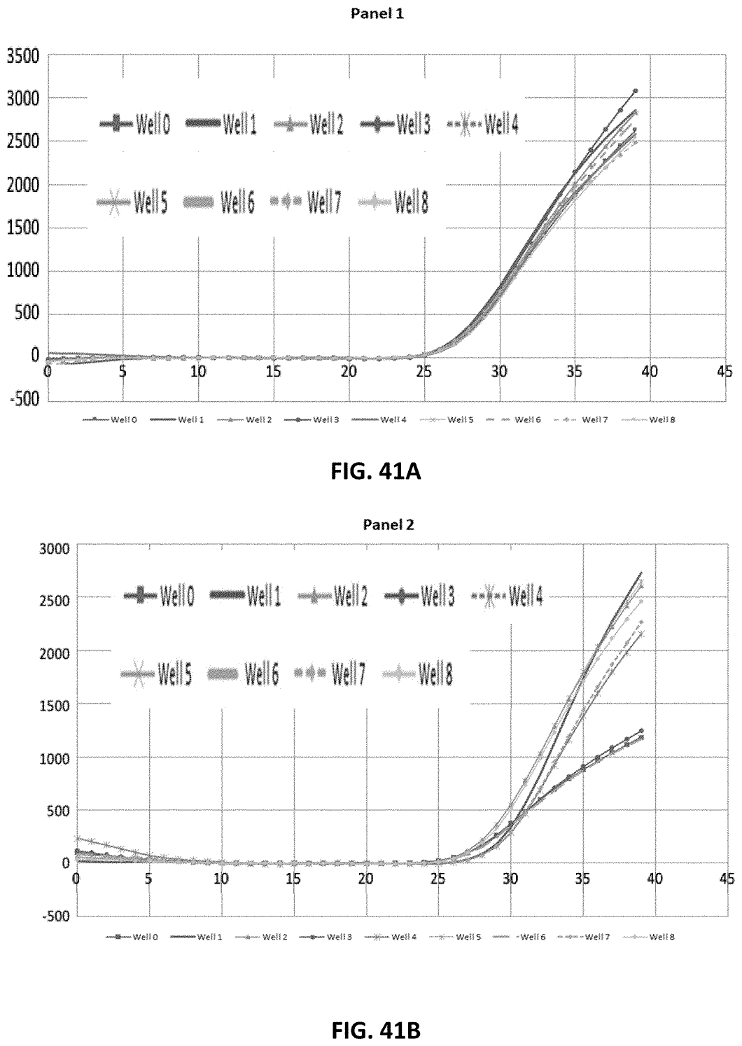

FIG. 41A and FIG. 41B show the PCR results of the samples within the sample preparation cartridge of FIG. 40A or FIG. 40B. The samples were prepared and analyzed using the sample preparation device and analytic device described herein.

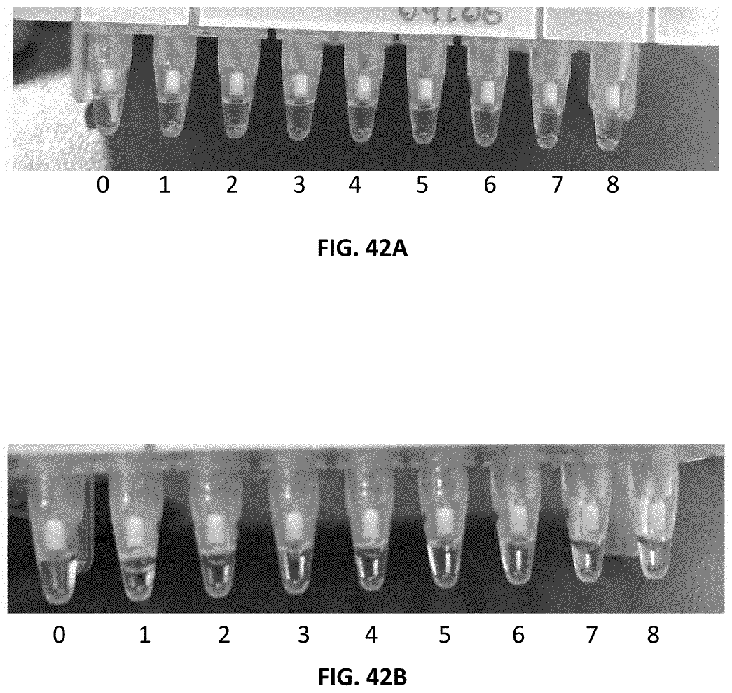

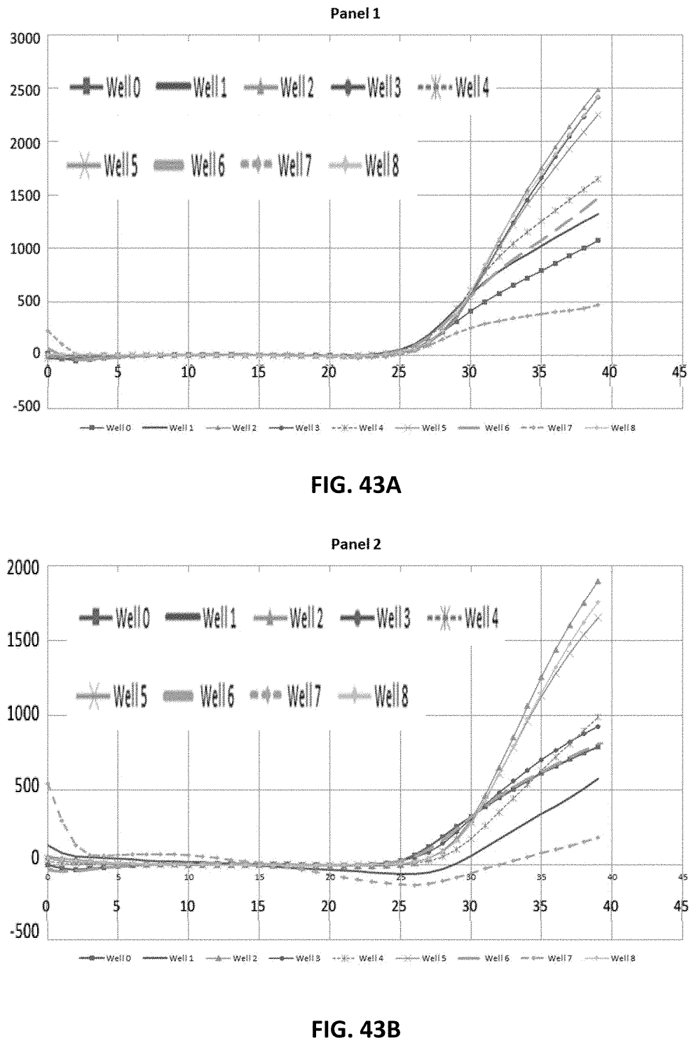

FIG. 42A shows an example sample preparation cartridge having an array of assay tubes filled with liquid samples with gas bubbles near bottom of the assay tubes. In this example, the sample preparation cartridge do not comprise swellable particles within the inflow conduits. The numbers indicate well numbers. FIG. 42B shows an image of the same sample preparation cartridge of FIG. 42A after performing PCR or thermo-cycling. In this example, significant fluid loss was observed. The PCR/Thermal cycling resulted in large amounts of fluid loss in various cuvettes. The gas in the assay tubes can expand and displace fluid volume into upper channels where it is lost. Starting from on the right: wells 0, 1, 4, 6, and 7 had net fluid loss. PCR results (see FIG. 43A and FIG. 43B) showed low performance in each of those affected wells on both channels and panel assays.

FIG. 43A and FIG. 43B show the PCR results of the samples within the sample preparation cartridge of FIG. 42A or FIG. 42B. The samples were prepared and analyzed using the sample preparation device and analytic device described herein. In this example, PCR results show poor performance associated with the problems caused by the air bubbles within the assay tubes.

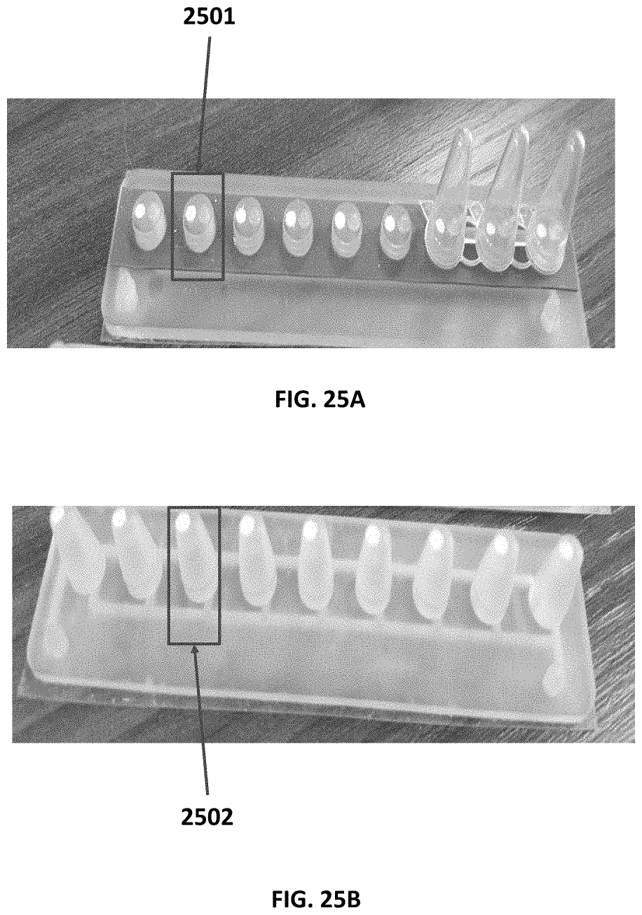

In some embodiments, two or more caps can have varying thicknesses causing the cap to extend into an assay tube, thereby affecting the maximum working volume of the assay tube. Example assay tube caps and assay tubes are shown in FIGS. 25A and 25B. FIG. 25A shows assay tube caps having a lesser thickness 2501 as compared to assay tube caps having a greater thickness 2502 shown in FIG. 25B. Generally, the greater the thickness of a cap (e.g., causing the cap to extend further into the assay tube) the lower the maximum working volume of the assay tube. This can be beneficial for small volume samples. In some cases, the thickness of the assay tube cap may be at least about 0.1 mm, about 0.2 mm, about 0.3 mm, about 0.4 mm, about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3 mm, about 4 mm, about 5 mm, about 6 mm, about 7 mm, about 8 mm, about 9 mm, about 10 mm or greater than about 10 mm. In some cases, the working volume of an assay tube may not be reduced. In some cases, the working volume of the assay tube may be reduced by at least about 1% about 2%, about 3% about 4%, about 5%, about 10%, about 15%, about 20%, about 25%, about 50%, about 75% or more than about 75%. Increasing the thickness of the cap reduces the distance between the bottom of the assay tube and the end of the conduit through which a sample is deposited into the assay tube; this can affect the mixing of the sample as a droplet falls into liquid already in the assay tube, as described above.

### Sample Preparation Devices

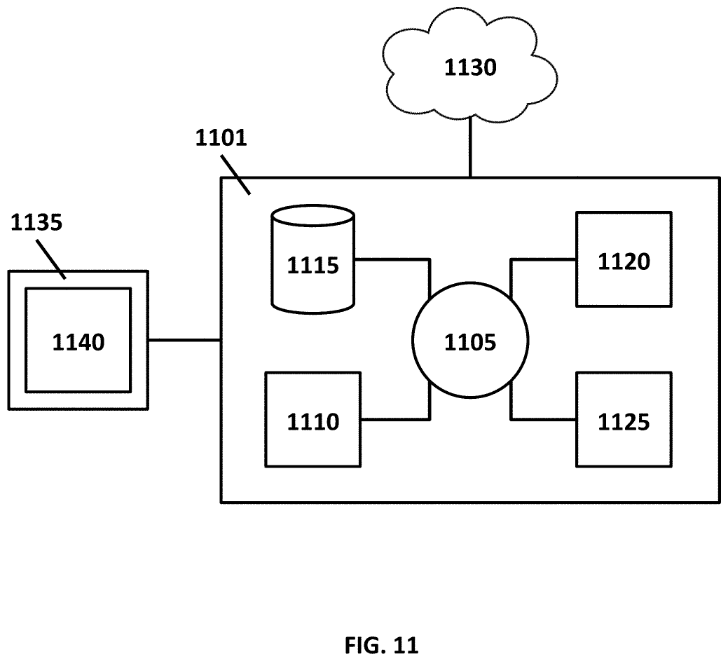

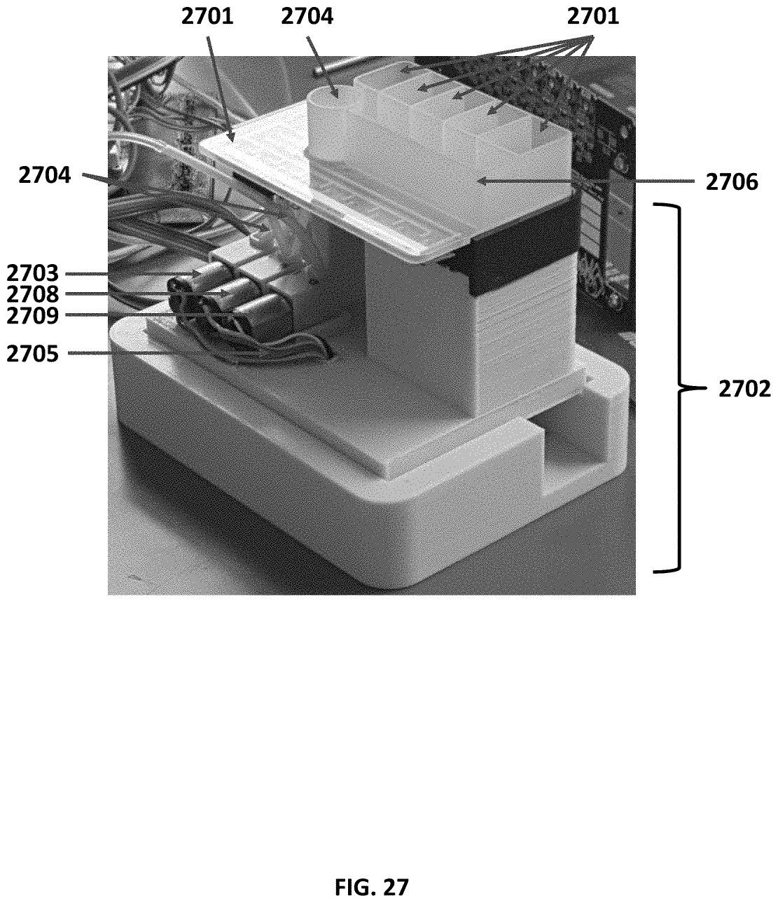

The sample preparation cartridge can be one component of a larger system which may comprise a sample preparation device for transferring fluids from one chamber to another chamber or an assay tube, and a computer based interface for controlling the device and/or interpretation of the data derived from the device. The sample preparation device can include a variety of mechanical elements (e.g., pumps and/or valves), and other computer-controlled systems. An example system is shown in FIG. 27. The sample preparation cartridge includes a housing comprising various chambers. A sample preparation cartridge 2701 is docked to a sample preparation device 2702 having pumps 2703, 2708, and 2709 and/or valves (not shown) to control the transfer of fluid between two or more chambers, including a reagent chamber 2701, a sample chamber 2704, and a waste chamber 2706 (e.g., from a reagent chamber to a sample chamber). Once docked, conduits leading from one or more sample chambers become fluidly connected 2704 (e.g., via tubing or channels) to one or more pumps and/or valves, thereby allowing the pumps and/or valves to control the flow of fluid from one chamber to another. The pumps and/or valves may be controlled wirelessly or using an electrical connection 2705 by one or more computer control systems, as shown in FIG. 11. In some cases, the sample preparation device described herein is a sample preparation unit within a system for sample processing and analyzing. The sample preparation unit can be within a same housing of an analysis unit (e.g., the analytic device described herein).

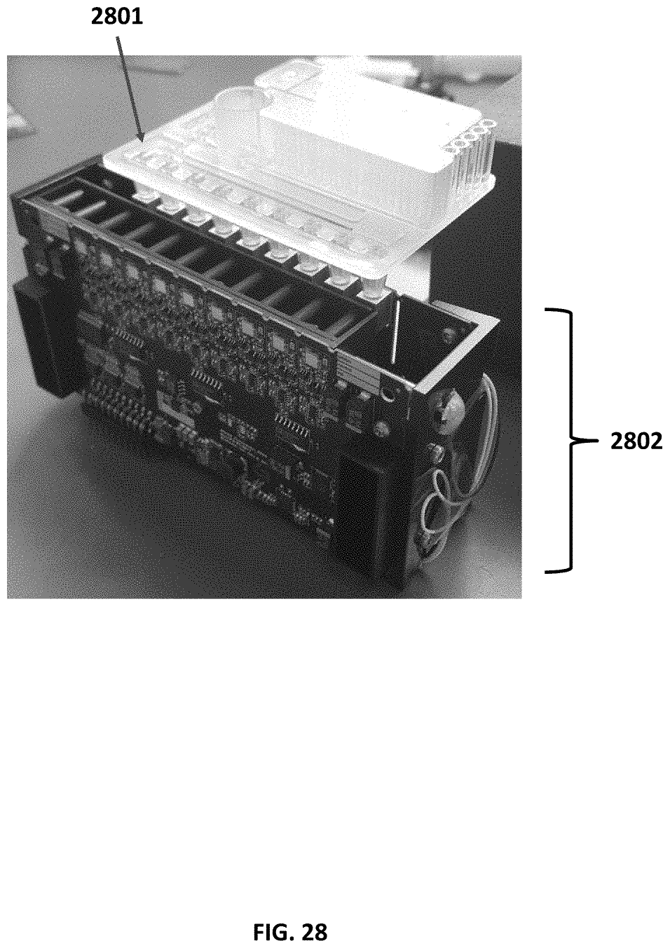

The sample preparation cartridge can be used with the analytic device as described herein for sample processing or analysis. FIG. 28 shows a sample preparation cartridge 2801 with assay tubes docked to an analytic device 2802 capable of performing an assay (e.g., polymerase chain reaction and/or detection of a target nucleic acid) on the sample in the assay tube.

The sample preparation cartridge may include information stored in a radiofrequency identification (RFID) unit or memory. The information may include a barcode that may uniquely identify the sample being processed, routines for processing the sample, or information about a user of the cartridge. Alternatively, the sample preparation cartridge may not include any RFID unit or memory. In some embodiments, the sample preparation may include a printed barcode or alpha-numeric code that may uniquely identify the sample being processed, routines for processing the sample, or information about a user of the cartridge.

The sample preparation device may comprise one or more fluid flow units. The fluid flow unit can be in fluid communication with a conduit and can be configured to subject a reagent to flow from a chamber (or well) to another chamber (or well). The sample preparation device may comprise at least 1, at least 2, at least 3, at least 4, at least 5, at least 6, at least 7, at least 8, at least 9, at least 10, at least 20, at least 30, at least 40, or more fluid flow units. The fluid flow unit can comprise a pump or a compressor. In some cases, the fluid flow unit is a pump or a compressor. In some cases, the fluid flow unit can comprise at least 1, at least 2, at least 3, at least 4, at least 5, at least 6, at least 7, at least 8, at least 9, at least 10, at least 20, at least 30, at least 40, or more pumps or compressors.

Pumps may be employed to generate pressure within the conduits to draw fluid from one chamber to another chamber or to generate bubbles within a chamber to induce mixing of a liquid in the chamber. A pump can be disposed along a conduit, or along tubing connecting one conduit opening to another. The pressure applied by the pump can be intermittent (e.g., a peristaltic pump) or continuous (e.g., a dynamic pump or velocity pump). A variety of devices may be employed. Non-limiting examples of pumps that may be used include a positive displacement pump, a gear pump, a screw pump, a rotary vane pump, a reciprocating pump, a plunger pump, a diaphragm pump, a piston pump, a rotary lobe pump, a progressive cavity pump, a rotary gear pump, a piston pump, a hydraulic pump, a peristaltic pump, a rope pump, a flexible impeller pump, an impulse pump, a velocity pump, a radial flow pump, a mixed-flow pump, an educator-jet pump, a gravity pump, a steam pump, and a valveless pump.

In some cases, the pump is a multi-directional pump. A multi-directional pump can be used to control fluid flow in two or more directions or two more modes of operation (e.g., each mode providing a different pressure or pressure drop). For example, the pump can be a bi-directional pump. The bi-directional pump may supply positive or negative pressure (or pressure drop). The bi-directional pump can control fluid flow in two opposite directions. The pump pressure can be controlled or changed over time while operating the systems or performing the methods described herein. As another example, the pump can operate at multiple modes of operation, such as a first mode in which a first pressure drop is applied and a second mode in which a second pressure drop is applied. The first pressure drop and/or second pressure drop may each yield a positive pressure. As an alternative, the first pressure drop and/or second pressure drop may each yield a negative pressure. As another alternative, the first pressure drop may yield a positive pressure and the second pressure drop may yield a negative pressure.

A multi-directional pump can supply increased or decreased pressure relative to a reference (e.g., ambient pressure). The systems provided herein can further comprise a pressure sensor included in or connected to the pump. The pressure sensor can measure the pressure of gas or liquid flowing in the conduit coupled to the pump. Such measurement can be used to regulate the pump—for example, with a pressure change, pumping may be terminated. In some cases, the pump pressure sensor monitors pressure of waste pump (e.g., P2 in FIG. 22D). In some cases, the pump pressure sensor monitors pressure of buffer pump (or reagent pump, e.g., P1 in FIG. 22D or FIG. 22E). In some cases, the pump pressure sensor monitors pressure of reaction pump (or sample pump, e.g., P3 in FIG. 22D). In some cases, the pump pressure sensor monitors pressure of drying pump (e.g., P4 in FIG. 22E).

Pumps of the present disclosure may be configured to supply various pressures or pressure drops. The pressure may be positive pressure or negative pressure. In some examples, a pump (e.g., multi-directional pump) may supply a pressure drop in a range of −50 kPa to 50 kPa, −40 kPa to 40 kPa, −20 kPa to 20 kPa, −10 kPa to 10 kpa, −5 kPa to 5 kPa, or −2 kPa to 2 kPa. The pressure may be greater than or equal to about 0.01 kPa, 0.1 kPa, 1 kPa, 2 kPa, 5 kPa, 10 kPa, 20 kPa, 30 kPa, 40 kPa, 50 kPa, 100 kPa, or greater. The pressure may be less than or equal to about 100 kPa, 50 kPa, 40 kPa, 30 kPa, 20 kPa, 10 kPa, 5 kPa, 2 kPa, 1 kPa, 0.1 kPa, 0.01 kPa, or less.

In some cases, a single pump may be fluidly coupled to (or capable of generating a pressure in) a single conduit. For example, a pump can be disposed along a conduit between a sample chamber and a waste chamber to pump a sample from the sample chamber to the waste chamber. In another example, a pump can be disposed along a conduit downstream of the assay tube to draw a sample from the sample chamber to the assay tube (e.g., the assay tube can be fluidly connected to the sample chamber via an additional conduit. In some cases, a single pump may be fluidly coupled to (or capable of generating a pressure in) multiple conduits simultaneously. For example, a pump can be disposed along a primary conduit, where one end of the primary conduit branches into multiple secondary conduits, each of which is fluidly connected to a chamber.

In another aspect, the present disclosure provides a system comprising a first pump and a second pump in fluid communication with a first fluid flow path. The first pump and the second pump can be multi-directional pumps (e.g., bi-directional pumps). The first pump and the second pump can be configured to subject fluid in the first fluid flow path to flow along a first direction and a second direction. The second direction may be different than the first direction.

For example, the first pump can supply positive pressure to flow a fluid along a first direction. In such instance, the second pump can supply negative pressure to drive the fluid along the first direction. Next, the first pump can supply negative pressure and the second pump can supply positive pressure to flow the fluid along the second direction, which may be opposite to the first direction.

Such system can include at least 1, 2, 3, 4, 5, 6, 7, 8, 9, 10, 20, 30, 40, 50, or more multi-directional pumps. In some cases, the fluid flow path may include valves at least 1, 2, 3, 4, 5, 6, 7, 8, 9, 10, 20, or more valves. As an alternative, the fluid flow path may not include any valves in the fluid flow path.

The fluid flow path may be a channel or conduit. For example, the fluid flow path may be a channel in a polymeric, metallic or composite substrate.

One or more valves may be employed, particularly when a single pump is used to apply a draw pressure to multiple chambers. In the example above, a pump can be disposed along a primary conduit, where one end of the primary conduit branches into multiple secondary conduits, each of which is fluidly connected to a chamber. A valve can be disposed along one or more secondary branches, thereby regulating a pressure applied by a pump on the chamber. A person having skill in the art will appreciate that a variety of valves may be used. Non-limiting examples of valves that may be used include a ball valve, a butterfly valve, a ceramic disc, a clapper valve, a check valve, a choke valve, a diaphragm valve, a gate valve, a globe valve, a knife valve, a needle valve, a pinch valve, a piston valve, a plug valve, a poppet valve, a spool valve, a thermal expansion valve, a pressure reducing valve, a sampling valve, and a safety valve. In some embodiments, the valve can be a one way valve. In some embodiments, the valve can be a two-way valve. In some embodiments, the valve can be a three-way valve. In some embodiments, the valve can be a four-way valve. In some embodiments, a system described herein may not comprise a valve.

Sensors may also be implemented to monitor performance of the sample preparation cartridges and systems. For example, pressure sensors may be used to detect movement of a fluid through one or more conduits of the sample preparation cartridge. In another example, may be used to detect a leak or contamination. In yet another example, optical or electrical sensors may be used to detect a level or amount of fluid within a chamber or conduit. Non-limiting examples of sensors that may be used include a pressure sensor, a moisture sensor, a magnetic sensor, a strain gauge, a force sensor, an inductive sensor, a resistive sensor, a capacitive sensor, an optical sensor, and any combination thereof.

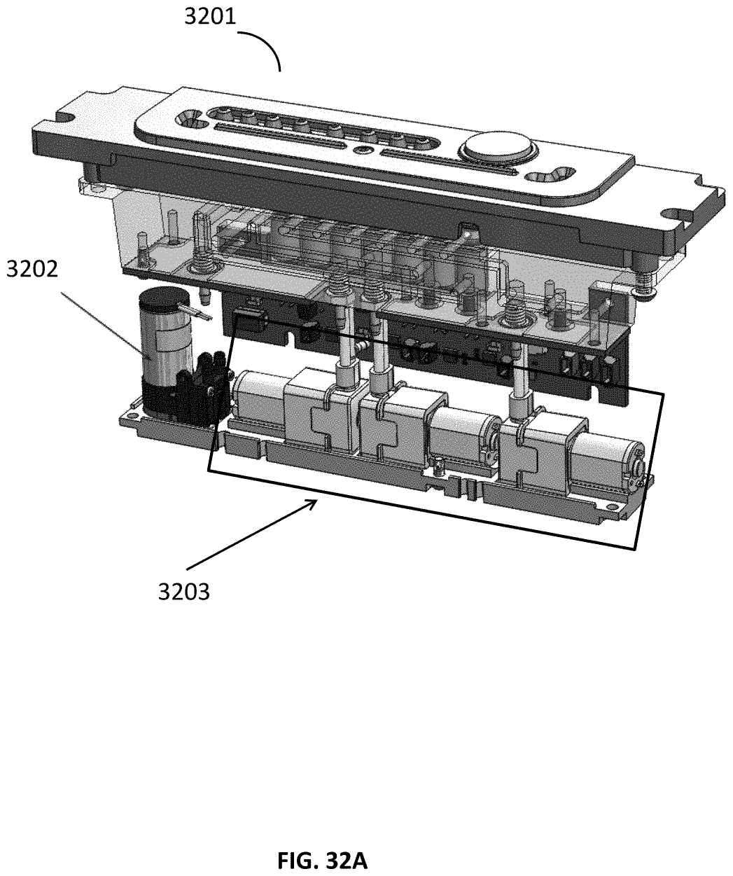

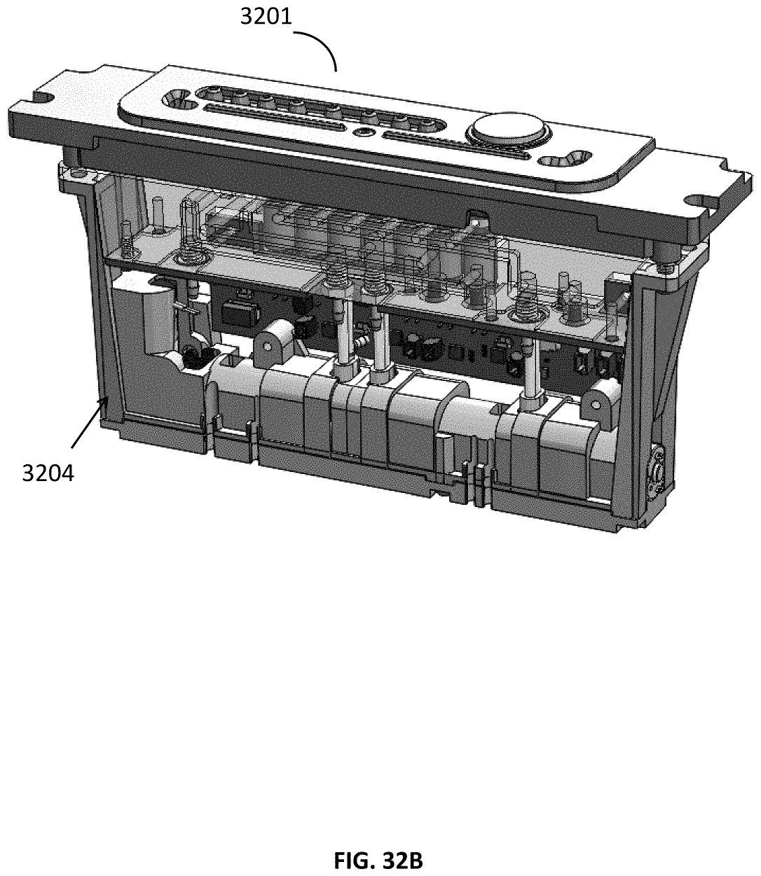

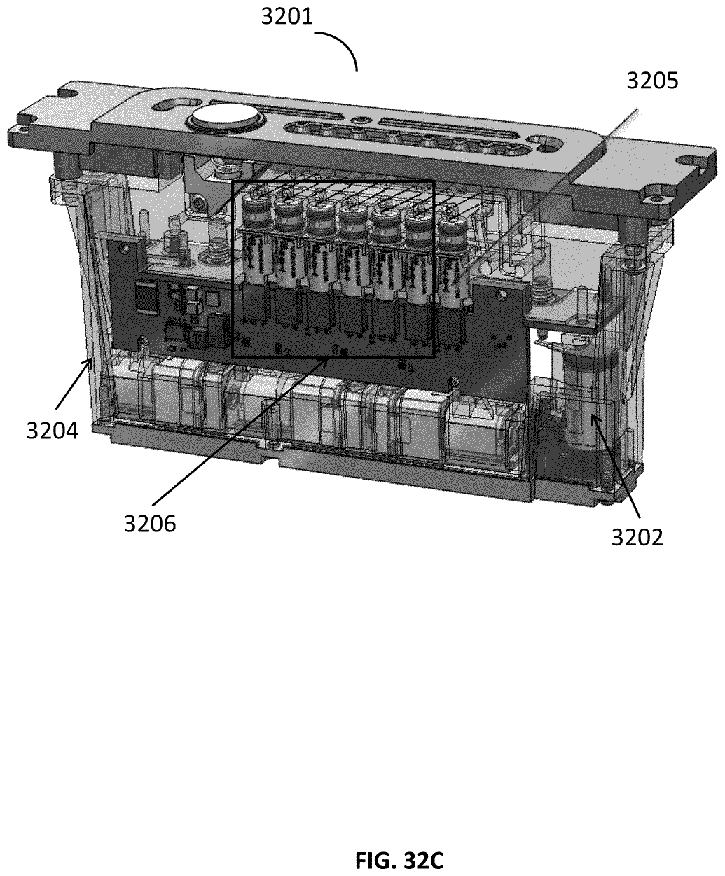

The sample preparation device may comprise a pump for drying at least one chamber of the sample preparation cartridge. The pump can be a diaphragm pump. The pump can be a unidirectional pump. The pump can be a peristaltic pump. FIGS. 32A-32C show an example sample preparation device assembly. In this example, the sample preparation device is configured according to the configuration in FIG. 22E, where a fourth pump is used for drying the chamber. The fourth pump can be a diaphragm pump. FIG. 32A shows a front view of the example sample preparation device 3201 having a diaphragm pump 3202 installed. The diaphragm pump 3202 can be used for drying chambers within a sample preparation cartridge. The sample preparation device 3201 also comprises three additional pumps 3203 for controlling fluid exchanges within the device. FIG. 32B shows a front view of the example sample preparation device assembly 3201 with a case 3204 to cover the pumps shown in FIG. 32A. FIG. 32C shows a back view of the example sample preparation device assembly 3201 shown in FIGS. 32A and 328. The sample preparation device 3201 comprises an additional valve 3205 in connection with the diaphragm pump 3202. The case 3204 is shown transparent in order to show the pumps within the case 3204. The sample preparation device also comprises additional six valves 3206 to control fluid flow to or from the reagent chambers as shown in FIG. 22E.

In some cases, the sample preparation device or system comprises at least 2, 3, 4, 5, 6, 7, 8, 9, 10, or more peristaltic pumps. In some cases, the sample preparation device or system comprises at least one diaphragm pump for drying at least one chamber of the sample preparation cartridge. For example, the at least one chamber of the sample preparation cartridge can be a waste chamber. In some cases, the sample preparation device or system comprises three peristaltic pumps and one diaphragm pump for drying, in some cases, the sample preparation device or system comprises two peristaltic pumps for controlling fluid flow within the sample preparation cartridge and an additional pump (e.g., a peristaltic pump or a diaphragm pump) for pumping waste from a sample chamber to the waste chamber and for drying the waste chamber.

### Assays

An assay may comprise nucleic acid amplification. For example, any type of nucleic acid amplification reaction may be used to amplify a target nucleic acid and generate an amplified product. Moreover, amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction, ligase chain reaction, asymmetric amplification, rolling circle amplification, and multiple displacement amplification (MDA). The amplified product may be DNA. In cases where a target RNA is amplified, DNA may be obtained by reverse transcription of the RNA and subsequent amplification of the DNA may be used to generate an amplified DNA product. The amplified DNA product may be indicative of the presence of the target RNA in the biological sample. In cases where DNA is amplified, various DNA amplification methods may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). DNA amplification may be linear. Alternatively, DNA amplification may be exponential. DNA amplification may be achieved with nested PCR, which may improve sensitivity of detecting amplified DNA products. Nucleic acid amplification may be isothermal. Non-limiting examples of isothermal nucleic acid amplification methods include helicase-dependent amplification, nicking enzyme amplification, recombinase polymerase amplification, loop-mediated isothermal amplification, and nucleic acid sequence based amplification.

Nucleic acid amplification reactions may be conducted in assay tubes in parallel. Nucleic acid amplification reactions may be conducted, for example, by including reagents necessary for each nucleic acid amplification reaction in a reaction vessel to obtain a reaction mixture and subjecting the reaction mixture to conditions necessary for each nucleic amplification reaction. Reverse transcription amplification and DNA amplification may be performed sequentially, such as, for example, performing reverse transcription amplification on RNA to generate complementary DNA (cDNA), and subsequently subjecting the cDNA to DNA amplification (e.g., PCR) to amplify the cDNA.

A nucleic acid sample may be amplified using reagents directed to a given target, such as, for example, a primer having sequence complementarity with a target sequence. After multiple heating and cooling cycles, any amplification products may be detected optically, such as using fluorophores. Fluorophore-labeled primers or hybridization probes and/or fluorescent dyes that bind to DNA maybe excited, and an emitted fluorescence detected. Detection may comprise analyzing fluorescence emission from a dye and calculating the ratio of fluorophore emission to dye emission. A primer may comprise a fluorophore and a quencher. In some cases, a tertiary structure of an unbound primer may be such that a quencher may be in close enough proximity to a fluorophore to prevent excitation of the fluorophore and/or the detection of an emission signal from the fluorophore.

In one example, a fluorescent DNA dye, such as SYBR Green I, may be added to a mixture containing a target nucleic acid and at least one amplification primer. In other examples, an amplification primer may be a linear single-stranded oligonucleotide that is extendable by a DNA polymerase and that is labeled with an excitable fluorophore. Upon performing an amplification reaction, such as, e.g., PCR, that includes annealing and extending the labeled primer, the fluorophore may be excited and a resultant emission detected during the amplification reaction (e.g., real-time detection) or following completion of the amplification reaction (e.g., an end-point detection at the conclusion of the amplification reaction or during a subsequent thermal analysis (melting curve)). Unincorporated primers may not fluoresce.

A wide range of fluorophores and/or dyes may be used in primers according to the present disclosure. Available fluorophores include coumarin; fluorescein; tetrachlorofluorescein; hexachlorofluorescein; Lucifer yellow; rhodamine; BODIPY; tetramethylrhodamine; Cy3; Cy5; Cy7; eosine; Texas red; SYBR Green I; SYBR Gold; 5-FAM (also called 5-carboxyfluorescein; also called Spiro(isobenzofuran-1(3H), 9′-(9H)xanthene)-5-carboxylic acid, 3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); DABCYL (4-((4-(dimethylamino)phenyl)azo)benzoic acid) Cy5 (Indodicarbocyanine-5) Cy3 (Indo-dicarbocyanine-3); BODIPY FL (2,6-dibromo-4,4-difluoro-5,7-dimethyl-4-bora-3a,4a-diaza-s-indacene-3-proprionic acid); Quasar-670 (Bioreseach Technologies); CalOrange (Bioresearch Technologies); and Rox as well as suitable derivatives thereof. Combination fluorophores such as fluorescein-rhodamine dimers may also be suitable. Fluorophores may be chosen to absorb and emit in the visible spectrum or outside the visible spectrum, such as in the ultraviolet or infrared ranges. Suitable quenchers may also include DABCYL and variants thereof, such as DABSYL, DABMI and Methyl Red. Fluorophores may also be used as quenchers, because they tend to quench fluorescence when touching certain other fluorophores. Preferred quenchers may be chromophores such as DABCYL or malachite green, or fluorophores that may not fluoresce in the detection range when the probe is in the open conformation.

Allele-discriminating probes useful according to the invention also include probes that bind less effectively to a target-like sequence, as compared to a target sequence. The change in the level of fluorescence in the presence or absence of a target sequence compared to the change in the level of fluorescence in the presence or absence of a target-like sequence may provide a measure of the effectiveness of binding of a probe to a target or target-like sequence.

DNA generated from reverse transcription of the RNA may be amplified to generate an amplified DNA product. Any suitable number of nucleic acid amplification reactions may be conducted. In some cases, at least 1, 2, 3, 4, 5, 6, 7, 8, 9, 10, 11, 12, 13, 14, 15, 16, 17, 18, 19, 20, or more nucleic acid amplification reactions are conducted.

For example, a target nucleic acid (e.g., target RNA, target DNA) may be extracted or released from a biological sample during heating phases of nucleic acid amplification. In the case of a target RNA, for example, the biological sample comprising the target RNA may be heated and the target RNA released from the biological sample. The released target RNA may begin reverse transcription (via reverse transcription amplification) to produce complementary DNA. The complementary DNA may then be amplified.

Primer sets directed to a target nucleic acid may be utilized to conduct nucleic acid amplification reaction. Primer sets may comprise one or more primers. For example, a primer set may comprise at least 1, 2, 3, 4, 5, 6, 7, 8, 9, 10, or more primers. A primer set may comprise primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

In cases in which a plurality of assay tubes is used, the plurality of assay tube may include the same primers or primer sets, or different primers or primer sets. Each assay tube may be directed to a different target, or at least a subset of the assay tubes may be directed to the same target.

For example, a primer set may be directed to a target RNA. The primer set may comprise a first primer that may be used to generate a first strand of nucleic acid product that is complementary to at least a portion the target RNA. In the case of a reverse transcription reaction, the first strand of nucleic acid product may be DNA. The primer set may also comprise a second primer that may be used to generate a second strand of nucleic acid product that is complementary to at least a portion of the first strand of nucleic acid product. In the case of a reverse transcription reaction conducted with DNA amplification, the second strand of nucleic acid product may be a strand of nucleic acid (e.g., DNA) product that is complementary to a strand of DNA generated from an RNA template.

Any suitable number of primer sets may be used. For example, at least about 1, 2, 3, 4, 5, 6, 7, 8, 9, 10, or more primer sets may be used. Where multiple primer sets are used, one or more primer sets may each correspond to a particular nucleic acid amplification reaction or amplified product.

A DNA polymerase may also be used. Any suitable DNA polymerase may be used, including commercially available DNA polymerases. A DNA polymerase may refer to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products, and derivatives thereof. A “hot start” polymerase may be used, e.g., in an amplification reaction. For certain “hot start” polymerases, a denaturation step at about 94° C.-95° C. for about 2 minutes to 10 minutes may be used, which may change the thermal profile based on different polymerases.

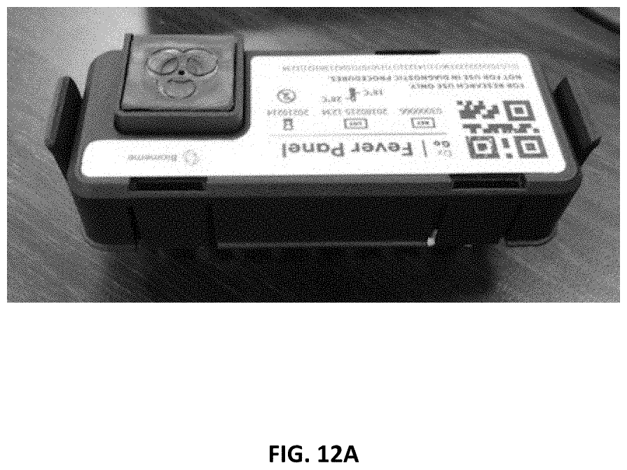

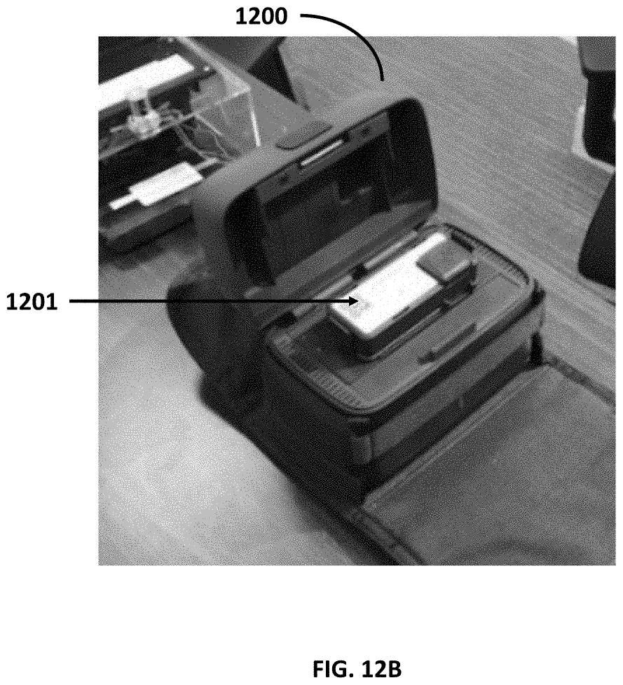

The reagents used for assays (e.g., thermocycling reactions or nucleic acid amplifications) can be provided in a reagent cartridge. The reagent cartridge can be premixed or prepacked. The reagent cartridge can be prepacked and ready for use. The reagent cartridge can be configured for different targets, for example, by containing primers specific for a given target or given targets. For example, the reagent cartridge can be configured for targeting microorganisms that cause a disease. In some embodiments, the reagent cartridge is configured for targeting nucleic acids from one or more microorganisms that cause fever or flu. In some embodiments, the reagent cartridge is configured for targeting nucleic acids from one or more viruses that cause fever or flu. In some embodiments, the reagent cartridge is configured for targeting nucleic acids from one or more microorganisms that cause an infectious disease. In some embodiments, the reagent cartridge is configured for targeting one or more microorganisms present in a sample. In some embodiments, the reagent cartridge is configured for targeting one or more microorganisms present in an environmental sample. The reagent cartridge can comprise a chamber for sample loading. An example cartridge is shown in FIG. 12A. The example cartridge 1201 can be inserted into the housing 1200 of the analytic device, for example, as shown in FIG. 12B.

The reagent cartridge can be stable and have a long shelf life. For example, the reagent cartridge can be stable at ambient condition or have a shelf life of at least about 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, or 30 months. For another example, the reagent cartridge can be stable at ambient condition or have a shelf life of at least 1 year, 1.5 years, 2 years, 2.5 years, 3 years, 4 years, 5 years, or longer.

In some cases, the reagent used for assays can be divided into two parts, a dry part and a wet (e.g., liquid) part. The dry part can be provided in a reagent cartridge as described herein. The wet part can be provided in the device during an assay. The dry part and the wet part can be mixed in the device when performing an assay.

In some embodiments, the wet part can be provided in a reagent cartridge as described herein. The dry part can be provided in the device during an assay. The dry part and the wet part can be mixed in the device when performing an assay.

In some embodiments, both the dry part and the wet part can be provided in a reagent cartridge without contacting or mixing with each other. In some embodiments, both the dry part and the part can be provided in separate reagent cartridges.

In some embodiments, the dry part and the wet part can be premixed before inserting into the device. In some embodiments, the dry part and the wet part can be inserted into the device and then mixed in the device.

When a wet reagent is provided in a reagent cartridge, the reagent cartridge can be sealed. In some embodiments, the reagent cartridge containing the wet reagent can be sealed by laser welding. Other methods to seal the reagent cartridge include, but are not limited to, using foil, membrane, film, or valve.

Using the device and reagent described in the present disclosure, the assay can be performed in various conditions. For example, the assay can be performed in various vibration conditions, dust levels, humidity levels, or altitudes. In some embodiments, the assay can be performed at normal ambient condition. For example, the normal ambient condition may have a temperature of about 25° C. and a pressure of about 100 kilopascal (kPa). In some other embodiments, the assay can be performed in a condition deviated from a normal ambient condition. In some cases, the assay can be performed at a pressure of at least 10 kPa, 20 kPa, 30 kPa, 40 kPa, 50 kPa, 60 kPa, 70 kPa, 80 kPa, 90 kPa, 100 kPa, 105 kPa, 110 kPa, 120 kPa, 130 kPa, or more. In some cases, the assay can be performed at a pressure of at most 70 kPa, 60 kPa, 50 kPa, 40 kPa, 30 kPa, 20 kPa, or 10 kPa. In some cases, the assay can be performed at an altitude above sea level. The altitude above sea level can be at least 500 feet, 1000 feet, 1500 feet, 2000 feet, 2500 feet, 3000 feet, 3500 feet, 4000 feet, 4500 feet, 5000 feet, 6000 feet, 7000 feet, 8000 feet, 9000 feet, 10000 feet, 15000 feet, 20000 feet, 30000 feet, 40000 feet, 50000 feet, or more. The assay described herein may be performed in space.

The assay described herein can be performed at various humidity levels. As used herein, absolute humidity (units are grams of water vapor per cubic meter volume of air) is a measure of the actual amount of water vapor in the air, regardless of the air's temperature. The higher the amount of water vapor, the higher the absolute humidity. For example, a maximum of about 30 grams of water vapor can exist in a cubic meter volume of air with a temperature of about 85° F. As used herein, relative humidity, expressed as a percent, is a measure of the amount of water vapor that air is holding compared to the amount it can hold at a specific temperature. Warm air can possess more water vapor (moisture) than cold air. For example, a relative humidity of 50% means that the air holds on that day (at a specific temperature) about 50% of the water needed for the air to be saturated. Saturated air has a relative humidity of 100%. In some embodiments, the assay can be performed at a humidity level with a relative humidity of at least 10%, 20%, 30%, 40%, 50%, 60%, 80%, 70%, 90%, 95%, 98%, or more.

### Computer Systems

The present disclosure provides computer systems that are programmed to implement methods of the disclosure. FIG. 11 shows a computer system 1101 that is programmed or otherwise configured to analyze a sample. The computer system 1101 may regulate some aspects of the analytic device of the present disclosure, such as, for example, movement of a moving carriage, heating or cooling of a heating block, and/or activation/deactivation of an excitation source or detector. The computer system may control of the temperature of a heating block (e.g., through activation of a resistive heater or fan). The computer system 1101 may be integrated into the analytic device of the present disclosure and/or include an electronic device of a user or a computer system that is remotely located with respect to the electronic device. The electronic device may be a mobile electronic device.

A computer system provided herein may regulate some aspects of the sample preparation device of the present disclosure. For example, the computer system 1101 can regulate various aspects of the sample preparation device of the present disclosure, such as, for example, activation of a valve or pump to transfer a reagent or sample from one chamber to another. In some aspects, the computer system can regulate which reagents or samples are mixed together, or the rate at which a sample or reagent is transferred from one chamber to another chamber.

The computer system 1101 includes a central processing unit (CPU, also “processor” and “computer processor” herein) 1105, which may be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 may be a data storage unit (or data repository) for storing data. The computer system 1101 may be operatively coupled to a computer network (“network”) 1130 with the aid of the communication interface 1120. The network 1130 may be the Internet, an internet and/or extranet, or an intranet and/or extranet that is in communication with the Internet. The network 1130 in some cases is a telecommunication and/or data network. The network 1130 may include one or more computer servers, which may enable distributed computing, such as cloud computing. The network 1130, in some cases with the aid of the computer system 1101, may implement a peer-to-peer network, which may enable devices coupled to the computer system 1101 to behave as a client or a server.

The CPU 1105 may execute a sequence of machine-readable instructions, which may be embodied in a program or software. The instructions may be stored in a memory location, such as the memory 1110. The instructions may be directed to the CPU 1105, which may subsequently program or otherwise configure the CPU 1105 to implement methods of the present disclosure. Examples of operations performed by the CPU 1105 may include fetch, decode, execute, and writeback.

The CPU 1105 may be part of a circuit, such as an integrated circuit. One or more other components of the system 1101 may be included in the circuit. In some cases, the circuit is an application specific integrated circuit (ASIC).

The storage unit 1115 may store files, such as drivers, libraries and saved programs. The storage unit 1115 may store user data, e.g., user preferences and user programs. The computer system 1101 in some cases may include one or more additional data storage units that are external to the computer system 1101, such as located on a remote server that is in communication with the computer system 1101 through an intranet or the Internet.

The computer system 1101 may communicate with one or more remote computer systems through the network 1130. For instance, the computer system 1101 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1101 via the network 1130.

Methods as described herein may be implemented by way of machine (e.g., computer processor) executable code stored on an electronic storage location of the computer system 1101, such as, for example, on the memory 1110 or electronic storage unit 1115. The machine executable or machine readable code may be provided in the form of software. During use, the code may be executed by the processor 1105. In some cases, the code may be retrieved from the storage unit 1115 and stored on the memory 1110 for ready access by the processor 1105. In some situations, the electronic storage unit 1115 may be precluded, and machine-executable instructions are stored on memory 1110.

The code may be pre-compiled and configured for use with a machine having a processor adapted to execute the code, or may be compiled during runtime. The code may be supplied in a programming language that may be selected to enable the code to execute in a pre-compiled or as-compiled fashion.

Aspects of the systems and methods provided herein, such as the computer system 1101, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 may include or be in communication with an electronic display 1135 that comprises a user interface (UI) 1140 for providing, for example, a current stage of processing of a sample (e.g., a particular step, such as a lysis step, that is being performed). Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 1105.

Methods and systems of the present disclosure may be combined with or modified by other methods or systems, such as, for example, those described in U.S. Pat. No. 9,579,655, which is entirely incorporated herein by reference.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-174. (canceled)

175. A method for analyzing a biological sample, comprising:
(a) activating a portable analytic device comprising:
(i) a housing;
(ii) at least one monolithic heating block within said housing, wherein said at least one monolithic heating block comprises a plurality of recesses configured to receive a plurality of assay tubes, wherein an assay tube of said plurality of assay tubes comprises said biological sample;
(iii) at least one heating unit in thermal communication with said at least one monolithic heating block, which at least one heating unit provides thermal energy to said assay tube through said monolithic heating block;
(iv) an excitation source configured to provide excitation energy;
(v) a movable carriage comprising an excitation filter and an emission filter, wherein said movable carriage is configured to translate to bring said excitation filter and said emission filter to a first position in alignment with a light path that provides excitation energy from said excitation source to said assay tube;
(vi) a power supply disposed within said housing, said power supply configured to provide power to said at least one heating unit, said movable carriage, and said excitation source; and
(vii) a processing unit comprising a circuit within said housing, wherein said processing unit is configured to communicate with a mobile electronic device external to said housing;
(b) receiving by said processing unit instructions from said mobile electronic device external to said housing for processing said biological sample in said assay tube;
(c) in response to said instructions, directing said at least one heating unit to provide thermal energy to said monolithic heating block to provide heat to said biological sample within said assay tube; and
(d) upon moving said movable carriage to said first position corresponding to said assay tube, directing said excitation source to expose said biological sample within said assay tube to excitation energy through said light path.

176. The method of claim 175, wherein moving said movable carriage to said first position corresponding to said assay tube comprises aligning said light path with said assay tube.

177. The method of claim 175, further comprising, subsequent to (d), detecting emission from said biological sample within said assay tube, which emission is indicative of a presence or absence, or a relative amount, of a target molecule within said biological sample.

178. The method of claim 175, wherein said movable carriage comprises a plurality of light paths.

179. The method of claim 175, wherein said portable analytic device further comprises an actuator for moving said movable carriage from said first position to a second position.

180. The method of claim 179, wherein:
(a) in said first position, said light path is aligned with said assay tube and capable of directing said excitation source to expose said biological sample within said assay tube to a first excitation energy; and
(b) in said second position, a second light path of a plurality of light paths is aligned with said assay tube and capable of directing said excitation source to expose said biological sample within said assay tube to a second excitation energy.

181. The method of claim 180, wherein said first excitation energy has a first wavelength, and said second excitation energy has a second wavelength.

182. The method of claim 175, further comprising receiving instructions at said processing unit from said mobile electronic device, said instructions comprising at least one temperature at which said at least one monolithic heating block is maintained.

183. The method of claim 175, further comprising extracting from said biological sample one or more nucleic acids.

184. The method of claim 175, wherein said biological sample comprises one or more members selected from the group consisting of a blood sample, a plant sample, a water sample, a soil sample, and a tissue sample.

185. The method of claim 175, wherein said biological sample contains or is suspected of containing a target nucleic acid molecule, and wherein said instructions comprise a target temperature(s) and number of heating and cooling cycles for conducting a nucleic acid amplification reaction on said target nucleic acid molecule, under conditions sufficient to yield amplification product(s) indicative of a presence or relative amount of said target nucleic acid molecule.

186. The method of claim 175, further comprising a data exchange unit that communicates with said mobile electronic device, wherein said data exchange unit (i) receives said instructions from said mobile electronic device, or (ii) provides results to said mobile electronic device upon processing said biological sample.

187. A portable analytic device for processing a biological sample, comprising:
a housing;
at least one monolithic heating block within said housing, wherein said at least one monolithic heating block comprises a plurality of recesses configured to receive a plurality of assay tubes;
at least one heating unit in thermal communication with said at least one heating block, which at least one heating unit is configured to provide thermal energy to said assay tube through said at least one monolithic heating block;
a movable carriage comprising an optical filter, wherein said movable carriage is configured to translate to bring said optical filter in alignment with a light path that provides excitation energy from an excitation source to said assay tube; and
a power supply disposed within said housing, said power supply configured to provide power to said at least one heating unit, said movable carriage, and said excitation source.

188. The portable analytic device of claim 187, further comprising a processing unit comprising a circuit within said housing, wherein said processing unit is configured to (i) direct said movable carriage to translate and/or (ii) direct said excitation source to provide said excitation energy.

189. The portable analytic device of claim 188, wherein said processing unit is operatively coupled to said at least one heating unit and/or said excitation source, and wherein said processing unit is configured to communicate with a mobile electronic device external to said housing.

190. The portable analytic device of claim 189, wherein said processing unit is configured to:
a. receive instructions from said mobile electronic device external to said housing for processing said biological sample in said assay tube; and
b. in response to said instructions, (i) direct said at least one heating unit to provide thermal energy to said at least one monolithic heating block to provide heat to said assay tube, and (ii) direct said excitation source to expose said assay tube to excitation energy.

191. The portable analytic device of claim 190, wherein said instructions comprise a temperature of said at least one heating unit and/or a duration that said at least one heating unit is held at said temperature.

192. The portable analytic device of claim 189, further comprising a communication unit that provides wireless communication between said processing unit and said mobile electronic device.

193. The portable analytic device of claim 187, wherein said actuator comprises a motor.

194. The portable analytic device of claim 187, further comprising an optical detector disposed within said housing, said optical detector configured to detect emission energy from said biological sample within said assay tube.
